SEQUENCE LISTING (94 sequences)

SEQ ID NO: 1 - RNA, 942 nt, synthetic construct
  source 1..942: mol_type = other RNA
atgccgctggagcagcgatcccaacattgcaaaccagaggaaggcctcgaggcaagaggtgaggccctggggctcgtcggagctcaggcaccggcaacggaggagcaagaagcggcctctagcagctccacactcgtcgaagtgacactcggcgaggtccccgctgcggaatctcccgatcctccgcaaagccctcaaggggcttcctccctccccactactatgaactatccgttgtggagccaaagctatgaggattcctcaaaccaagaggaggaaggcccctccacattcccggacctcgaatctgagttccaggcagcgctcagcaggaaagtggcggaacttgtacagtttttgttgcttaaataccgggctcgggagccagtcacgaaagccgagatgttaggcagcgtggtgggtaattggcagtacttcttccccgtcatcttttccaaggcttcctcttctttgcaactcgtttttggcatagagcttatggaagtagacccgacaggtcatctttacatttttgcaacgtgtctcggcctcagttatgacgggctgctcggtgacaatcaaattatgcctaaggccggactgcttatcatcgtcttggctataattgcccgagagggcgactgtgctcccgaagagaaaatatgggaggagctctccgtgctggaggtatttgagggaagagaagatagcatactgggagaccctaagaaattgcttacacaacactttgtacaagagaactatttggagtatcgacaggtccccggatctgacccagcatgttatgagttcctgtgggggccaagggcactcgtagagacgtcatatgtcaaggtgctgcaccacatggtcaaaataagcggcggcccccacataagctacccacccctccatgaatgggttttgagggagggtgaagaa

SEQ ID NO: 2 - RNA, 1611 nt, synthetic construct
  source 1..1611: mol_type = other RNA
atgaaatcttacaacgtcctccccctagcctatatctcccttttcctgatgctgttttatcaggtttgggctcagtttccacgagagtgtgccaatattgaggctctgagacgtggggtgtgttgcccagacctgctcccttcctctggaccggggactgacccttgtggctcatcatcaggaagaggcaggtgtgtggctgtgattgcagactcccgaccccacagccgccattatccccacgatggtaaagatgaccgagaagcctggcctctgaggttctttaatagaacatgtcagtgcaatgataatttctcaggacacaactgtgggacttgccgtcctgggtggagaggagctgcatgcaaccagaaaattctcacagtcaggagaaatcttctagacttaagtccagaagaaaagagccactttgtcagggccttggatatggcgaagcgcacaactcaccctcaatttgtcattgccacaaggaggttagaagacatactgggaccagatggcaacacaccacaatttgagaacatttccgtttataactactttgtttggacacactattattcagtcaaaaaaaccttcctcgggacaggacaggaaagctttggggatgtggatttctctcacgaaggacccgcttttctcacatggcacaggtaccatctgctgcagctggagagagacatgcaggagatgctgcaggagccttctttctcccttccttactggaattttgcaactgggaaaaacgtctgcgatgtctgcactgatgacttgatgggatccagaagcaacttcgattctactcttataagccccaactctgtcttttctcaatggagagtggtctgtgaatccttggaagagtacgataccctgggaacactttgtaacagcactgagggtggaccaatcaggagaaacccagctggaaatgtagggagaccagcagtgcagcgtcttcctgagccacaggatgtcactcagtgcttggaggtccgtgtatttgacacacctcctttttattccaattctacagacagttttcgaaatacagtggaaggttacagtgctcccacgggaaaatatgaccctgctgttcgaagccttcacaacctggcccacctcttcctgaatggaacgggaggacaaacccatttgtctcccaatgatcctatttttgtcctcctgcacactttcactgatgcggtctttgacgaatggctaaggaggtataacgccgatatttctaccttcccgttggaaaacgcacctattggacataacaggcaatacaacatggtgccattctggcctccagttaccaacacagaaatgtttgttactgctccagacaatctgggatatatgtatgaagttcaatggccaggtcaggagtttactgtatctgaaatcattaccattgctgtagtggctgcgttgttacttgtagctgccattttcggggttgcttcttgtctgatccgttctagaagcaccaagaatgaagccaaccagcctctcctcactgatcactatcaacgctatgctgaggactatgaggagctcccgaatcctaaccactccatggtc

SEQ ID NO: 3 - protein, 9 aa, Homo sapiens
KCDICTDEY

SEQ ID NO: 4 - protein, 9 aa, Homo sapiens
YMDGTMSQV

SEQ ID NO: 5 - protein, 9 aa, Homo sapiens
MLLAYLYQL

SEQ ID NO: 6 - protein, 9 aa, Homo sapiens
AFLPWHRLF

SEQ ID NO: 7 - protein, 9 aa, Homo sapiens
SEIWRDIDF

SEQ ID NO: 8 - protein, 9 aa, Homo sapiens
YLEPGPVTA

SEQ ID NO: 9 - protein, 9 aa, Homo sapiens
KTWGQUWQV

SEQ ID NO: 10 - protein, 9 aa, Homo sapiens
ITDQVPFSV

SEQ ID NO: 11 - protein, 10 aa, Homo sapiens
VLYRYGSFSV

SEQ ID NO: 12 - protein, 10 aa, Homo sapiens
LLDGTATLRL

SEQ ID NO: 13 - protein, 9 aa, Homo sapiens
ALLAVGATK

SEQ ID NO: 14 - protein, 9 aa, Homo sapiens
MLGTHTMEV

SEQ ID NO: 15 - protein, 9 aa, Homo sapiens
LIYRRRLMK

SEQ ID NO: 16 - protein, 9 aa, Homo sapiens
ALNFPGSQK

SEQ ID NO: 17 - protein, 9 aa, Homo sapiens
AAGIGILTV

SEQ ID NO: 18 - protein, 9 aa, Homo sapiens
ILTVILGVL

SEQ ID NO: 19 - protein, 9 aa, Homo sapiens
MSLQRQFLR

SEQ ID NO: 20 - protein, 9 aa, Homo sapiens
SVYDFFVWL

SEQ ID NO: 21 - protein, 9 aa, Homo sapiens
LLGPGRPYR

SEQ ID NO: 22 - protein, 9 aa, Homo sapiens
YLSGANLNL

SEQ ID NO: 23 - protein, 9 aa, Homo sapiens
KIFGSLAFL

SEQ ID NO: 24 - protein, 10 aa, Homo sapiens
VMAGVGSPYV

SEQ ID NO: 25 - protein, 9 aa, Homo sapiens
IISAVVGIL

SEQ ID NO: 26 - protein, 9 aa, Homo sapiens
LLHETDSAV

SEQ ID NO: 27 - protein, 9 aa, Homo sapiens
ALFDIESKV

SEQ ID NO: 28 - protein, 9 aa, Homo sapiens
EADPTGHSY

SEQ ID NO: 29 - protein, 9 aa, Homo sapiens
SLFRAVITK

SEQ ID NO: 30 - protein, 9 aa, Homo sapiens
SAYGEPRKL

SEQ ID NO: 31 - protein, 9 aa, Homo sapiens
KMVELVHFL

SEQ ID NO: 32 - protein, 10 aa, Homo sapiens
YLOLVFGIEV

SEQ ID NO: 33 - protein, 9 aa, Homo sapiens
EVDPIGHLY

SEQ ID NO: 34 - protein, 9 aa, Homo sapiens
FLWGPRALV

SEQ ID NO: 35 - protein, 10 aa, Homo sapiens
MEVDPIGHLY

SEQ ID NO: 36 - protein, 9 aa, Homo sapiens
AARAVFLAL

SEQ ID NO: 37 - protein, 8 aa, Homo sapiens
YRPRPRRY

SEQ ID NO: 38 - protein, 9 aa, Homo sapiens
VLPDVFIRC

SEQ ID NO: 39 - protein, 9 aa, Homo sapiens
QLSLLMWIT

SEQ ID NO: 40 - protein, 9 aa, Homo sapiens
SLLMWITQC

SEQ ID NO: 41 - protein, 10 aa, Homo sapiens
ASGPGGGAPR

SEQ ID NO: 42 - protein, 10 aa, Homo sapiens
QDLTMKYQIF

SEQ ID NO: 43 - protein, 10 aa, Homo sapiens
  VARIANT 1: note = E or absent
XAYGLDFYIL

SEQ ID NO: 44 - protein, 9 aa, Homo sapiens
SYLDSGIHF

SEQ ID NO: 45 - protein, 9 aa, Homo sapiens
ETVSEQSNV

SEQ ID NO: 46 - protein, 9 aa, Homo sapiens
FPSDSWCYF

SEQ ID NO: 47 - protein, 9 aa, Homo sapiens
EEKLIVVLF

SEQ ID NO: 48 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 443 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 449 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 449 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 451 aa, synthetic construct
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 449 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 222 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 64 - protein, 456 aa, synthetic construct
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 220 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERK

SEQ ID NO: 66 - protein, 446 aa, synthetic construct
VQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 226 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 68 - protein, 446 aa, synthetic construct
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - RNA, 7561 nt, Venezuelan equine encephalitis virus
  source 1..7561: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 72 - protein, 51 aa, Venezuelan equine encephalitis virus
RELPVLDSAAFNVECFKKYACNNEYWETFKENPIRLTEENVVNYITKLKGP

SEQ ID NO: 73 - protein, 41 aa, Venezuelan equine encephalitis virus
TQMRELPVLDSAAFNVECFKKYACNNEYWETFKENPIRLTE

SEQ ID NO: 74 - protein, 536 aa, Venezuelan equine encephalitis virus
MKAITARRILQGLGHYLKAEGKVECYRTLHPVPLYSSSVNRAFSSPKVAVEACNAMLKENFPTVASYCIIPEYDAYLDMIDGASCCLDTASFCPAKLRSFPKKHSYLEPTIRSAVPSAIQNTLQNVLAAATKRNCNVTQMRELPVLDSAAFNVECFKKYACNNEYWKTFKENPIRLTEENVINYITKLKGPKAAALYAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTAMMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILPELCKAVESRYETVGTSVIVMAMATLASSVKSFSYLRGAPITLYG

SEQ ID NO: 75 - RNA, 8806 nt, synthetic construct
  source 1..8806: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgccgctggagcagcgatcccaacattgcaaaccagaggaaggcctcgaggcaagaggtgaggccctggggctcgtcggagctcaggcaccggcaacggaggagcaagaagcggcctctagcagctccacactcgtcgaagtgacactcggcgaggtccccgctgcggaatctcccgatcctccgcaaagccctcaaggggcttcctccctccccactactatgaactatccgttgtggagccaaagctatgaggattcctcaaaccaagaggaggaaggcccctccacattcccggacctcgaatctgagttccaggcagcgctcagcaggaaagtggcggaacttgtacagtttttgttgcttaaataccgggctcgggagccagtcacgaaagccgagatgttaggcagcgtggtgggtaattggcagtacttcttccccgtcatcttttccaaggcttcctcttctttgcaactcgtttttggcatagagcttatggaagtagacccgacaggtcatctttacatttttgcaacgtgtctcggcctcagttatgacgggctgctcggtgacaatcaaattatgcctaaggccggactgcttatcatcgtcttggctataattgcccgagagggcgactgtgctcccgaagagaaaatatgggaggagctctccgtgctggaggtatttgagggaagagaagatagcatactgggagaccctaagaaattgcttacacaacactttgtacaagagaactatttggagtatcgacaggtccccggatctgacccagcatgttatgagttcctgtgggggccaagggcactcgtagagacgtcatatgtcaaggtgctgcaccacatggtcaaaataagcggcggcccccacataagctacccacccctccatgaatgggttttgagggagggtgaagaatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 76 - RNA, 9475 nt, synthetic construct
  source 1..9475: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgaaatcttacaacgtcctccccctagcctatatctcccttttcctgatgctgttttatcaggtttgggctcagtttccacgagagtgtgccaatattgaggctctgagacgtggggtgtgttgcccagacctgctcccttcctctggaccggggactgacccttgtggctcatcatcaggaagaggcaggtgtgtggctgtgattgcagactcccgaccccacagccgccattatccccacgatggtaaagatgaccgagaagcctggcctctgaggttctttaatagaacatgtcagtgcaatgataatttctcaggacacaactgtgggacttgccgtcctgggtggagaggagctgcatgcaaccagaaaattctcacagtcaggagaaatcttctagacttaagtccagaagaaaagagccactttgtcagggccttggatatggcgaagcgcacaactcaccctcaatttgtcattgccacaaggaggttagaagacatactgggaccagatggcaacacaccacaatttgagaacatttccgtttataactactttgtttggacacactattattcagtcaaaaaaaccttcctcgggacaggacaggaaagctttggggatgtggatttctctcacgaaggacccgcttttctcacatggcacaggtaccatctgctgcagctggagagagacatgcaggagatgctgcaggagccttctttctcccttccttactggaattttgcaactgggaaaaacgtctgcgatgtctgcactgatgacttgatgggatccagaagcaacttcgattctactcttataagccccaactctgtcttttctcaatggagagtggtctgtgaatccttggaagagtacgataccctgggaacactttgtaacagcactgagggtggaccaatcaggagaaacccagctggaaatgtagggagaccagcagtgcagcgtcttcctgagccacaggatgtcactcagtgcttggaggtccgtgtatttgacacacctcctttttattccaattctacagacagttttcgaaatacagtggaaggttacagtgctcccacgggaaaatatgaccctgctgttcgaagccttcacaacctggcccacctcttcctgaatggaacgggaggacaaacccatttgtctcccaatgatcctatttttgtcctcctgcacactttcactgatgcggtctttgacgaatggctaaggaggtataacgccgatatttctaccttcccgttggaaaacgcacctattggacataacaggcaatacaacatggtgccattctggcctccagttaccaacacagaaatgtttgttactgctccagacaatctgggatatatgtatgaagttcaatggccaggtcaggagtttactgtatctgaaatcattaccattgctgtagtggctgcgttgttacttgtagctgccattttcggggttgcttcttgtctgatccgttctagaagcaccaagaatgaagccaaccagcctctcctcactgatcactatcaacgctatgctgaggactatgaggagctcccgaatcctaaccactccatggtctgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 77 - protein, 314 aa, synthetic construct
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVQFLLLKYRAREPVTKAEMLGSVVGNWQYFFPVIFSKASSSLQLVFGIELMEVDPTGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 78 - protein, 537 aa, synthetic construct
MKSYNVLPLAYISLFLMLFYQVWAQFPRECANIEALRRGVCCPDLLPSSGPGTDPCGSSSGRGRCVAVIADSRPHSRHYPHDGKDDREAWPLRFFNRTCQCNDNFSGHNCGTCRPGWRGAACNQKILTVRRNLLDLSPEEKSHFVRALDMAKRTTHPQFVIATRRLEDILGPDGNTPQFENISVYNYFVWTHYYSVKKTFLGTGQESFGDVDFSHEGPAFLTWHRYHLLQLERDMQEMLQEPSFSLPYWNFATGKNVCDVCTDDLMGSRSNFDSTLISPNSVFSQWRVVCESLEEYDTLGTLCNSTEGGPIRRNPAGNVGRPAVQRLPEPQDVTQCLEVRVFDTPPFYSNSTDSFRNTVEGYSAPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYMYEVQWPGQEFTVSEIITIAVVAALLLVAAIFGVASCLIRSRSTKNEANQPLLTDHYQRYAEDYEELPNPNHSMV

SEQ ID NO: 79 - DNA, 6569 bp, synthetic construct
  source 1..6569: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacaagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaacccgtggtcagatccagatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 80 - RNA, 7862 nt, synthetic construct
  source 1..7862: mol_type = other RNA
  misc_difference 7562: note = a, c, g, or u
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagntgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 81 - RNA, 7862 nt, synthetic construct; identical to SEQ ID NO: 80
  source 1..7862: mol_type = other RNA
  misc_difference 7562: note = a, c, g, or u

SEQ ID NO: 82 - RNA, 7862 nt, synthetic construct
  source 1..7862: mol_type = other RNA
  misc_difference 7562: note = a, c, g, or u
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagntagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 83 - RNA, 7862 nt, synthetic construct; identical to SEQ ID NO: 80
  source 1..7862: mol_type = other RNA
  misc_difference 7562: note = a, c, g, or u

SEQ ID NO: 84 - RNA, 7862 nt, synthetic construct; identical to SEQ ID NO: 80
  source 1..7862: mol_type = other RNA
  misc_difference 7562: note = a, c, g, or u

SEQ ID NO: 85 - RNA, 11701 nt, synthetic construct
  source 1..11701: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 86 - RNA, 7862 nt, synthetic construct
  source 1..7862: mol_type = other RNA
  misc_difference 7565: note = a, c, g, or u
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccntaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 87 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 88 - RNA, 942 nt, Homo sapiens; identical to SEQ ID NO: 1
  source 1..942: mol_type = genomic RNA

SEQ ID NO: 89 - RNA, 8806 nt, synthetic construct; identical to SEQ ID NO: 75
  source 1..8806: mol_type = other RNA

SEQ ID NO: 90 - protein, 553 aa, Homo sapiens
MVQRLWVSRLLRHRKAQLLLVNLLTFGLEVCLAAGITYVPPLLLEVGVEEKFMTMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSA

SEQ ID NO: 91 - protein, 9 aa, Homo sapiens
AAGIGILTV

SEQ ID NO: 92 - protein, 9 aa, Homo sapiens
SYLDSGIHS

SEQ ID NO: 93 - protein, 9 aa, Homo sapiens
ETVSEESNV

SEQ ID NO: 94 - protein, 9 aa, Homo sapiens
EEKLSVVLF

(19) United States
(12) Patent Application Publication
(10) Pub. No.: US 2026/0048108 A1
(43) Pub. Date: Feb. 19, 2026

(54) CANCER THERAPY COMPOSITIONS AND USES THEREOF

(71) Applicant: HDT Bio Corp, Seattle, WA (US)

(72) Inventors: Steven Gregory Reed, Bellevue, WA (US); Darrick Albert Carter, Seattle, WA (US); Malcolm Scott Duthie, Sammamish, WA (US); Lars Peter Aksel Berglund, Seattle, WA (US); Jesse Hong-Sae Erasmus, Port Orchard, WA (US); Jiho Kim, Seattle, WA (US)

(21) Appl. No.: 19/380,218

(22) Filed: Nov. 5, 2025

Related U.S. Application Data
(63) Continuation of application No. 18/607,029, filed on Mar. 15, 2024, now Pat. No. 12,485,163.
Application No. 18/607,029 is a continuation of application No. PCT/US2022/076304, filed on Sep. 12, 2022.
(60) Provisional application No. 63/247,167, filed on Sep. 22, 2021.
(60) Provisional application No. 63/302,360, filed on Jan. 24, 2022.

(51) Int. Cl. A61K 39/00; A61K 9/127; A61K 9/48; A61K 9/50; A61K 9/51; C12N 15/86

(52) U.S. Cl. CPC A61K 39/001193; A61K 9/4858; A61K 9/5015; A61K 9/5115; A61K 9/5123; A61K 39/001156; A61K 39/001186; C12N 15/86; A61K 9/127; A61K 2039/53; A61K 2039/55555; A61K 2039/55566; A61K 2039/6075; C12N 2720/12143

(57) ABSTRACT

The disclosure provides compositions, methods of treatment, and methods of making and using compositions to deliver a nucleic acid to a subject. Compositions described herein include lipid carriers, optionally including an inorganic particle, capable of admixing with nucleic acids. Nucleic acids provided herein include those encoding for cancer antigens (full length proteins or fragments) as well as antibodies. Methods of using the compositions as a therapeutic vaccine for the treatment of a cancer are also provided.

### CROSS REFERENCE

This application is a continuation of U.S. patent application Ser. No. 18/607,029, filed Mar. 15, 2024, which is a continuation of International Application No. PCT/US2022/076304, filed Sep. 12, 2022, which claims the benefit of priority to U.S. Provisional Patent Application No. 63/247,167, filed Sep. 22, 2021, and U.S. Provisional Patent Application No. 63/302,360, filed Jan. 24, 2022, the contents of each of which are incorporated herein by reference in their entirety.

### SEQUENCE LISTING

The instant application contains a Sequence Listing which has been submitted electronically in xml format and is hereby incorporated by reference in its entirety. Said xml copy, created on Mar. 15, 2024, is named 201953-715301-SL.xml and is 199,986 bytes in size.

### BACKGROUND

Cancer is one of the most challenging human diseases to treat or prevent. Cancer cells evade the immune system by suppressing immune cell signaling, avoid programmed cell death, are resistant and adaptive to chemotherapeutic agents. Cancer cells evade the immune system by presenting as “self” and generate tolerizing signals not recognized by the immune system. In addition, solid cancer cells form tight junctions within the affected organ that resist the delivery and impair the efficacy of chemotherapeutic agents and cell therapies. Thus, there is a need for improved compositions and methods for the prevention and treatment of a broad range of cancers.

### BRIEF SUMMARY

Provided herein are compositions comprising: lipid nanoparticles, wherein the lipid nanoparticles comprise: surfactants, wherein the surfactants comprise: a cationic lipid; a hydrophilic surfactant; and a hydrophobic surfactant; and at least one nucleic acid, wherein the at least one nucleic acid comprises a sequence encoding for a cancer-associated protein region comprising a cell membrane-contacting domain or a functional fragment thereof. Further provided herein is a composition, wherein the cell membrane-contacting domain comprises a transmembrane-binding domain, an outer cell membrane-contacting domain, or an inner cell membrane-contacting domain. Further provided herein is a composition, wherein the cell membrane-contacting domain comprises a transmembrane-binding domain, an outer cell membrane-contacting domain, and an inner cell membrane-contacting domain. Further provided herein is a composition, wherein the cancer-associated protein is prostein. Further provided herein is a composition, wherein the at least one nucleic acid comprises a region encoding a sequence at least about 80%, 85%, 90%, 95%, 98%, or 99% identical to SEQ ID NO: 90. Further provided herein is a composition, wherein the at least one nucleic acid comprises a region encoding a sequence of SEQ ID NO: 90. Further provided herein is a composition, wherein the cancer-associated protein is a protein expressed by a solid cancer cell or a blood cancer cell. Further provided herein is a composition, wherein the blood cancer cell comprises a melanoma cancer cell, a prostate cancer cell, a colon cancer cell, an ovarian cancer cell, a breast cancer cell, or a pancreatic cancer cell. Further provided herein is a composition, wherein the at least one nucleic acid is in complex with the lipid nanoparticles to form nucleic acid-lipid nanoparticle complexes. Further provided herein is a composition, wherein the at least one nucleic acid further comprises sequence encoding for an RNA-dependent polymerase. Further provided herein is a composition, wherein the composition comprises a second nucleic acid that encodes for an RNA-dependent polymerase Further provided herein is a composition, wherein the RNA-dependent polymerase is a Venezuelan equine encephalitis virus (VEEV) RNA polymerase. Further provided herein is a composition, wherein is the sequence encoding the RNA-dependent polymerase comprises the nucleic acid sequence of SEQ ID NO: 71. Further provided herein is a composition, wherein the lipid nanoparticles comprise a hydrophobic core. Further provided herein is a composition, wherein lipids present in hydrophobic core are in liquid phase at 25° C. Further provided herein is a composition, wherein the lipid nanoparticles are characterized as having a z-average diameter particle size measurement of about 20 nm to about 80 nm when measured using dynamic light scattering. Further provided herein is a composition, wherein the hydrophobic core comprises liquid oil. Further provided herein is a composition, wherein the liquid oil is α-tocopherol, coconut oil, grapeseed oil, lauroyl polyoxylglyceride, mineral oil, monoacylglycerol, palmkernal oil, olive oil, paraffin oil, peanut oil, propolis, squalene, squalane, soy lecithin, soybean oil, sunflower oil, a triglyceride, or vitamin E. Further provided herein is a composition, wherein the triglyceride is capric triglyceride, caprylic triglyceride, a caprylic and capric triglyceride, a triglyceride ester, or myristic acid triglycerin. Further provided herein is a composition, wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis(azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″′,9″″′-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis(octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein is a composition, wherein the lipid nanoparticles comprise an inorganic particle. Further provided herein is a composition, wherein the inorganic particle is within the hydrophobic core. Further provided herein is a composition, wherein the inorganic particle comprises a metal. Further provided herein is a composition, wherein the metal comprises a metal salt, a metal oxide, a metal hydroxide, or a metal phosphate. Further provided herein is a composition, wherein the metal oxide comprises aluminum oxide, aluminum oxyhydroxide, iron oxide, titanium dioxide, or silicon dioxide. Further provided herein is a composition, wherein the hydrophobic surfactant is sorbitan monolaurate, sorbitan monopalmitate, sorbitan monostearate, sorbitan monooleate, or sorbitan trioleate. Further provided herein is a composition, wherein the hydrophilic surfactant is a polysorbate.

Provided herein are compositions lipid nanoparticles, wherein the lipid nanoparticles comprise: a surface comprising cationic lipids; and a hydrophobic core; and nucleic acids, wherein the nucleic acids comprise a sequence encoding for TRP-1 protein or a functional fragment thereof, and wherein the nucleic acids are complexed to the cationic lipids to form nucleic acid-lipid nanoparticle complexes. Further provided herein is a composition, wherein the nucleic acids comprise a sequence at least about 80%, 85%, 90%, 95%, 98%, or 99% identical to SEQ ID NO: 2. Further provided herein is a composition, wherein the nucleic acids comprise a sequence of SEQ ID NO: 2. Further provided herein is a composition, wherein the nucleic acids comprise a sequence at least about 80%, 85%, 90%, 95%, 98%, or 99% identical to SEQ ID NO: 76. Further provided herein is a composition, wherein the nucleic acids comprise a sequence of SEQ ID NO: 76. Further provided herein is a composition, wherein the nucleic acids encode an amino acid sequence that is at least about 80%, 85%, 90%, 95%, 98%, or 99% identical to SEQ ID NO: 78 or a functional fragment thereof. Further provided herein is a composition, wherein the nucleic acids encode an amino acid sequence that comprises SEQ ID NO: 78 or a functional fragment thereof. Further provided herein is a composition, further comprising a nucleic acid that encodes an RNA polymerase. Further provided herein is a composition, wherein the nucleic acids further comprise sequence encoding for an RNA polymerase. Further provided herein is a composition, wherein the RNA polymerase is a Venezuelan equine encephalitis virus (VEEV) RNA polymerase. Further provided herein is a composition, wherein the sequence encoding the RNA polymerase comprises the nucleic acid sequence of SEQ ID NO: 71. Further provided herein is a composition, wherein lipids present in hydrophobic core are in liquid phase at 25° C. Further provided herein is a composition, wherein the lipid nanoparticles are characterized as having a z-average diameter particle size measurement of about 20 nm to about 80 nm when measured using dynamic light scattering. Further provided herein is a composition, wherein the hydrophobic core comprises liquid oil. Further provided herein is a composition, wherein the liquid oil comprises α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, soy lecithin, soybean oil, sunflower oil, a triglyceride, or vitamin E. Further provided herein is a composition, wherein the triglyceride is capric triglyceride, caprylic triglyceride, a caprylic and capric triglyceride, a triglyceride ester, or myristic acid triglycerin. Further provided herein is a composition, wherein the cationic lipids comprise 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis(azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; $3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″,9″″′-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3; or N1,N3,N5-tris(3-(didodecylamino)propyl)benzene-1,3,5-tricarboxamide. Further provided herein is a composition, wherein the lipid nanoparticles comprise an inorganic particle. Further provided herein is a composition, wherein the inorganic particle is within the hydrophobic core. Further provided herein is a composition, wherein the inorganic particle comprises a metal. Further provided herein is a composition, wherein the metal comprises a metal salt, a metal oxide, a metal hydroxide, or a metal phosphate. Further provided herein is a composition, wherein the metal oxide comprises aluminum oxide, aluminum oxyhydroxide, iron oxide, titanium dioxide, or silicon dioxide. Further provided herein is a composition, further comprising a hydrophobic surfactant and a hydrophilic surfactant. Further provided herein is a composition, wherein the hydrophobic surfactant comprises sorbitan monolaurate, sorbitan monopalmitate, sorbitan monostearate, sorbitan monooleate, or sorbitan trioleate. Further provided herein is a composition, wherein the hydrophilic surfactant comprises polysorbate.

Provided here are compositions comprising a composition comprising: lipid nanoparticles, wherein the lipid nanoparticles comprise: a surface comprising cationic lipids; and a hydrophobic core; and nucleic acids, wherein the nucleic acids comprise a sequence encoding for MAGE-A1 protein or a functional fragment thereof, and wherein the nucleic acids are complexed to the cationic lipids to form nucleic acid-lipid nanoparticle complexes. Further provided herein is a composition, wherein the nucleic acids comprise a sequence at least about 80%, 85%, 90%, 95%, 98%, or 99% identical to SEQ ID NO: 1. Further provided herein is a composition, wherein the nucleic acids comprise a sequence of SEQ ID NO: 1. Further provided herein is a composition, wherein the nucleic acids comprise a sequence at least about 80%, 85%, 90%, 95%, 98%, or 99% identical to SEQ ID NO: 88. Further provided herein is a composition, wherein the nucleic acids comprise a sequence of SEQ ID NO: 88. Further provided herein is a composition, wherein the nucleic acids comprise a sequence at least about 80%, 85%, 90%, 95%, 98%, or 99% identical to SEQ ID NO: 75. Further provided herein is a composition, wherein the nucleic acids comprise a sequence of SEQ ID NO: 75. Further provided herein is a composition, wherein the nucleic acids comprise a sequence at least about 80%, 85%, 90%, 95%, 98%, or 99% identical to SEQ ID NO: 89. Further provided herein is a composition, wherein the nucleic acids comprise a sequence of SEQ ID NO: 89. Further provided herein is a composition, wherein the nucleic acids encode an amino acid sequence that is at least about 80%, 85%, 90%, 95%, 98%, or 99% identical to SEQ ID NO: 77 or a functional fragment thereof. Further provided herein is a composition, wherein the nucleic acids encode an amino acid sequence that comprises SEQ ID NO: 77 or a functional fragment thereof. Further provided herein is a composition, wherein the nucleic acids encode an amino acid sequence that is at least about 80%, 85%, 90%, 95%, 98%, or 99% identical to SEQ ID NO: 87 or a functional fragment thereof. Further provided herein is a composition, wherein the nucleic acids encode an amino acid sequence that comprises SEQ ID NO: 87 or a functional fragment thereof. Further provided herein is a composition, wherein comprising a nucleic acid that encodes an RNA polymerase. Further provided herein is a composition, wherein the nucleic acids further comprise sequence encoding for an RNA polymerase. Further provided herein is a composition, wherein the RNA polymerase is a Venezuelan equine encephalitis virus (VEEV) RNA polymerase. Further provided herein is a composition, wherein the sequence encoding the RNA polymerase comprises the nucleic acid sequence of SEQ ID NO: 71. Further provided herein is a composition, wherein lipids present in hydrophobic core are in liquid phase at 25° C. Further provided herein is a composition, wherein the lipid nanoparticles are characterized as having a z-average diameter particle size measurement of about 20 nm to about 80 nm when measured using dynamic light scattering. Further provided herein is a composition, wherein the hydrophobic core comprises liquid oil. Further provided herein is a composition, wherein the liquid oil comprises α-tocopherol, coconut oil, grapeseed oil, lauroyl polyoxylglyceride, mineral oil, monoacylglycerol, palmkernal oil, olive oil, paraffin oil, peanut oil, propolis, squalene, squalane, soy lecithin, soybean oil, sunflower oil, a triglyceride, or vitamin E. Further provided herein is a composition, wherein the triglyceride is capric triglyceride, caprylic triglyceride, a caprylic and capric triglyceride, a triglyceride ester, or myristic acid triglycerin. Further provided herein is a composition, wherein the cationic lipids comprise 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis (azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; $3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,?9″′″-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis(octadeca-9,12-dienoate); PEG2000-DMG, (R)-2,3-bis(myristoyloxy)propyl-1-(methoxy poly(ethylene glycol)2000) carbamate; TT3; or N1,N3,N5-tris(3-(didodecylamino)propyl)benzene-1,3,5-tricarboxamide. Further provided herein is a composition, wherein the lipid nanoparticles comprise an inorganic particle. Further provided herein is a composition, wherein the inorganic particle is within the hydrophobic core. Further provided herein is a composition, wherein the inorganic particle comprises a metal. Further provided herein is a composition, wherein the metal comprises a metal salt, a metal oxide, a metal hydroxide, or a metal phosphate. Further provided herein is a composition, wherein the metal oxide comprises aluminum oxide, aluminum oxyhydroxide, iron oxide, titanium dioxide, or silicon dioxide. Further provided herein is a composition, wherein comprising a hydrophobic surfactant and a hydrophilic surfactant. Further provided herein is a composition, wherein the hydrophobic surfactant comprises sorbitan monolaurate, sorbitan monopalmitate, sorbitan monostearate, sorbitan monooleate, or sorbitan trioleate. Further provided herein is a composition, wherein the hydrophilic surfactant comprises polysorbate.

Provided herein are compositions comprising a nucleic acid, wherein the nucleic acid comprises a sequence encoding for: a protein or a functional fragment thereof listed in Table 1; and an RNA polymerase complex region. Further provided herein is a composition, wherein the protein or functional fragment thereof is a cancer-associated protein. Further provided herein is a composition, wherein the protein or functional fragment thereof comprises an amino acid sequence referenced in Table 1. Further provided herein is a composition, wherein the protein or functional fragment thereof comprises an antibody or a functional fragment thereof. Further provided herein is a composition, wherein the antibody or the functional fragment thereof comprises an antibody listed in Table 2. Further provided herein is a composition, wherein the antibody comprises an immunoglobulin (Ig) molecule or a functional fragment thereof. Further provided herein is a composition, wherein the immunoglobulin molecule is an IgG, IgE, IgM, IgD, IgA, or an IgY isotype immunoglobulin molecule or a functional fragment thereof. Further provided herein is a composition, wherein the immunoglobulin molecule of the functional fragment comprises at least a fragment of an IgG1, an IgG2, an IgG3, an IgG4, an IgGA1, or an IgGA2 subclass immunoglobulin molecule. Further provided herein is a composition, wherein the antibody or functional fragment thereof specifically binds to a tumor antigen or a viral antigen. Further provided herein is a composition, wherein the antibody or functional fragment thereof is atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab. Further provided herein is a composition, wherein the RNA polymerase complex region is downstream of a subgenomic promoter from an alphavirus. Further provided herein is a composition, wherein RNA polymerase complex region encodes for an RNA-dependent RNA polymerase. Further provided herein is a composition, wherein the RNA-dependent RNA polymerase is Venezuelan equine encephalitis virus (VEEV) RNA polymerase. Further provided herein is a composition, wherein the sequence encoding for the RNA polymerase complex region comprises SEQ ID NO: 71. Further provided herein is a composition, further comprising a lipid nanoparticle for complexation to the nucleic acid. Further provided herein is a composition, wherein the composition is lyophilized. Further provided herein is a composition, wherein the composition is in a liquid, semi-liquid, solution, propellant, or powder dosage form. Further provided herein is a composition, wherein the composition is formulated as a suspension.

Provided herein are pharmaceutical compositions comprising a composition described herein and a pharmaceutically acceptable excipient. In some embodiments, the pharmaceutically acceptable excipient comprises water. In some embodiments, the pharmaceutically acceptable excipient comprises a sugar. In some embodiments, the sugar comprises sucrose.

Provided herein are methods of generating an immune response in a subject, the method comprising: administering to a subject a composition described herein, or a pharmaceutical composition described herein, thereby generating an immune response to a cancer-associated protein. Further provided herein are methods, wherein the composition is administered to the subject over at least two doses. Further provided herein are methods, wherein the at least two doses are administered at least about 28 days apart. Further provided herein are methods, wherein up to 5 μg, 10 μg 25 ug, or more of nucleic acid is present in the composition administered to the subject. Further provided herein are methods, wherein the composition is administered via intramuscular injection, intranasal administration, oral administration, subcutaneous administration, intratumoral administration, intrathecal administration, or intravenous injection. Further provided herein are methods, wherein the subject is a domesticated animal or farmed animal. Further provided herein are methods, wherein the subject is a mammal. Further provided herein are methods, wherein the subject is a human. Further provided herein are methods, wherein the subject has, is at risk for, or is suspected of having a cancer. Further provided herein are methods, wherein the subject has a solid tumor or a blood cancer. Further provided herein are methods, wherein the solid tumor is a carcinoma, a melanoma, or a sarcoma. Further provided herein are methods, wherein the blood cancer is lymphoma or leukemia. Further provided herein are methods, wherein the subject has, is at risk for developing, or is suspected of having a skin cancer. Further provided herein are methods, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are methods, wherein the subject has, is at risk for developing, or is suspected of having a pancreatic cancer. Further provided herein are methods, wherein the pancreatic cancer is a pancreatic adenocarcinoma, a pancreatic exocrine cancer, a pancreatic neuroendocrine cancer, an islet cell cancer, or a pancreatic endocrine cancer. Further provided herein are methods, wherein the subject has, is at risk for developing, or is suspected of having a colon cancer, a prostate cancer, an ovarian cancer, or a breast cancer. Further provided herein are methods, wherein the cancer expresses a TRP-1 protein, a prostein protein, a MAGE-A1 protein, a MAGE-A3 protein, or a combination thereof.

Provided herein are methods treating a cancer in a subject, the method comprising: receiving a biomarker report that indicates that a subject has a cancer; classifying the cancer based on the biomarker report; and administering to the subject the composition or pharmaceutical composition described herein. Further provided herein are methods, wherein the composition is administered to the subject over at least two doses. Further provided herein are methods, wherein the at least two doses are administered at least about 28 days apart. Further provided herein are methods, wherein up to 5 μg, 10 μg 25 ug, or more of nucleic acid is present in the composition administered to the subject. Further provided herein are methods, wherein the composition is administered via intramuscular injection, intranasal administration, oral administration, subcutaneous administration, intratumoral administration, intrathecal administration, or intravenous injection. Further provided herein are methods, wherein the subject is a domesticated animal or farmed animal. Further provided herein are methods, wherein the subject is a mammal. Further provided herein are methods, wherein the subject is a human. Further provided herein are methods, wherein the cancer is a solid cancer or a blood cancer. Further provided herein are methods, wherein the cancer expresses a TRP-1 protein, a prostein protein, a MAGE-A1 protein, a MAGE-A3 protein, or a combination thereof.

Provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the at least one nucleic acid comprises a sequence encoding for a cancer-associated protein.

Further provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the at least one nucleic acid comprises a sequence encoding for an antibody or a functional variant thereof.

Further provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the at least one nucleic acid comprises a sequence encoding for a cancer therapeutic antibody or a functional variant thereof.

Further provided herein are compositions, wherein the compositions comprise: a lipid carrier, wherein the lipid carrier comprises: liquid oil; an inorganic nanoparticle, wherein the inorganic nanoparticle comprises iron oxide present in an amount of about 0.2 mg/ml 12 nm iron oxide; and surfactants, wherein the surfactants comprise a cationic lipid; and at least one nucleic acid, wherein the nucleic acid comprises a sequence encoding for a cancer-associated protein sequence or functional variant thereof.

Further provided herein are compositions, wherein the compositions comprise: (a) a lipid carrier, wherein the lipid carrier is a nanoemulsion comprising: about 30 mg/mL DOTAP chloride; about 37.5 mg/ml squalene; about 37 mg/ml sorbitan monostearate; about 37 mg/ml polysorbate 80; about 10 mM sodium citrate; and about 0.2 mg Fe/ml 12 nm oleic acid-coated iron oxide nanoparticles; and (b) at least one nucleic acid, wherein the at least one nucleic acid comprises a sequence encoding for a cancer-associated protein sequence or functional variant thereof.

Further provided herein are compositions, wherein the compositions comprise: (a) a lipid carrier, wherein the lipid carrier is a nanoemulsion comprising: DOTAP chloride present in an amount of about 0.75 mg; squalene present in an amount of about 0.94 mg; sorbitan monostearate present in an amount of about 0.93 mg; polysorbate 80 present in an amount of about 0.93 mg; citric acid monohydrate present in an amount of about 1.05 mg; and oleic acid-coated iron oxide nanoparticles present in an amount of about 0.005 mg; and (b) at least one nucleic acid, wherein the at least one nucleic acid comprises a sequence encoding for a cancer-associated protein sequence or functional variant thereof.

Further provided herein are compositions, wherein the compositions comprise: a first nucleic acid comprising a sequence encoding for an RNA-dependent RNA polymerase; and a second nucleic acid comprising a sequence encoding for a cancer-associated protein sequence or functional variant thereof.

Further provided herein are compositions, wherein the compositions comprise: a first nucleic acid comprising a sequence encoding for an RNA-dependent RNA polymerase; and a second nucleic acid comprising a sequence encoding for a cancer-associated protein binding antibody or antibody fragment.

Further provided herein are compositions, wherein the compositions comprise: (a) a lipid carrier, wherein the lipid carrier is a nanoemulsion comprising a hydrophobic core, optionally one or more inorganic nanoparticles; and one or more lipids; and (b) at least one nucleic acid sequence, wherein the nucleic acid sequence comprises a sequence which encodes a sequence capable of expressing an antigen, wherein the antigen is a cancer-associated protein.

Further provided herein are vaccines, wherein the vaccines comprise: (a) a lipid carrier, wherein the lipid carrier is a nanoemulsion comprising a hydrophobic core, optionally one or more inorganic nanoparticles and one or more lipids; and (b) at least one nucleic acid sequence, wherein the nucleic acid sequence comprises a sequence which encodes a sequence capable of expressing an antigen, wherein the antigen is a cancer-associated protein.

Further provided herein are methods of generating an immune response in a subject, wherein the methods comprise: administering to said subject a composition comprising a lipid carrier, wherein the lipid carrier is a nanoemulsion comprising a hydrophobic core, one or more inorganic nanoparticles and one or more lipids, and at least one nucleic acid sequence, wherein the nucleic acid sequence comprises a sequence which encodes a sequence capable of expressing an antigen, wherein the antigen is a cancer-associated protein.

Further provided herein are compositions for immunoprotecting a subject, wherein the compositions comprise: a lipid carrier, wherein the lipid carrier is a nanoemulsion comprising a hydrophobic core, one or more inorganic nanoparticles and one or more lipids, and at least one nucleic acid sequence, wherein the nucleic acid sequence comprises a sequence which encodes a sequence capable of expressing an antigen, wherein the antigen is a cancer-associated protein.

Further provided herein are dried compositions, wherein the dried compositions comprise: a composition provided herein; and at least one cryoprotectant.

Further provided herein are compositions for prophylaxis of a cancer, the compositions comprising: a sorbitan fatty acid ester, an ethoxylated sorbitan ester, a cationic lipid, an immune stimulant, and at least one RNA encoding an antigen sequence or functional fragment thereof.

Further provided herein are compositions for prophylaxis of a cancer, the compositions comprising: sorbitan monostearate (e.g., SPAN® 60), polysorbate 80 (e.g., TWEEN® 80), DOTAP, an immune stimulant, and at least one RNA encoding an antigen sequence or functional fragment thereof.

Further provided herein are pharmaceutical compositions, wherein the pharmaceutical compositions comprise: a composition provided herein; and a pharmaceutically acceptable excipient. Further provided herein are pharmaceutical compositions, wherein the pharmaceutical compositions comprise: (a) a lipid carrier, wherein the lipid carrier is a nanoemulsion comprising a hydrophobic core, optionally one or more inorganic nanoparticles and one or more lipids; and (b) at least one nucleic acid sequence, wherein the nucleic acid sequence comprises a sequence which encodes a sequence capable of expressing an antigen, wherein the antigen is a cancer-associated protein.

Further provided herein are kits, wherein the kits comprise a composition provided herein.

Further provided herein are methods of generating an immune response in a subject, the methods comprise: administering to a subject a composition provided herein, thereby generating an immune response to a cancer-associated protein. Further provided herein are methods of generating an immune response in a subject, wherein the methods comprise: administering to a subject: (a) a lipid carrier, wherein the lipid carrier is a nanoemulsion comprising a hydrophobic core, optionally one or more inorganic nanoparticles and one or more lipids; and (b) at least one nucleic acid sequence, wherein the nucleic acid sequence comprises a sequence which encodes a sequence capable of expressing an antigen, wherein the antigen is a cancer-associated protein.

Further provided herein are methods of prophylactically immunizing a subject for a cancer, the methods comprise: administering to a subject a composition provided herein, thereby immunizing the subject to a cancer expressing a cancer-associated protein.

Further provided herein are methods of reducing the severity of a cancer, wherein the methods comprise: administering a composition comprising a lipid carrier, wherein the lipid carrier is a nanoemulsion comprising a hydrophobic core, optionally one or more inorganic nanoparticles and one or more lipids, and at least one nucleic acid sequence, wherein the nucleic acid sequence comprises a sequence which encodes a sequence capable of expressing an antigen, wherein the antigen is a cancer-associated protein.

Further provided herein are methods of immunoprotecting a subject, wherein the methods comprise: administering to the subject a lipid carrier, wherein the lipid carrier is a nanoemulsion comprising a hydrophobic core, one or more inorganic nanoparticles and one or more lipids, at least one nucleic acid sequence, wherein the nucleic acid sequence comprises a sequence which encodes a sequence capable of expressing an antigen, wherein the antigen is a cancer-associated protein.

Further provided herein are methods for preparing a lyophilized composition, wherein the methods comprising: obtaining a lipid carrier, wherein the lipid carrier is a nanoemulsion comprising a hydrophobic core, one or more inorganic nanoparticles and one or more lipids, incorporating at least one nucleic acid into the lipid carrier to form a lipid carrier-nucleic acid complex, wherein the nucleic acid has a sequence comprising an antigen sequence as set forth in one of SEQ ID NOS: 1-2, 75, 76, 88, or 89; adding at least one cryoprotectant to the lipid carrier-nucleic acid complex to form a formulation, and lyophilizing the formulation to form a lyophilized composition.

Further provided herein are methods for preparing a spray-dried composition, wherein the methods comprise: obtaining a lipid carrier, wherein the lipid carrier is a nanoemulsion comprising a hydrophobic core, one or more inorganic nanoparticles and one or more lipids, incorporating at least one nucleic acid into the lipid carrier to form a lipid carrier-nucleic acid complex, wherein the nucleic acid has a sequence comprising an antigen sequence as set forth in one of SEQ ID NOS: 1-2, 75, 76, 88, or 89; adding at least one cryoprotectant to the lipid carrier-nucleic acid complex to form a formulation, and spray drying the formulation to form a spray-dried composition.

The present invention also relates to a method for reconstituting a lyophilized composition comprising obtaining a lipid carrier, wherein the lipid carrier is a nanoemulsion comprising a hydrophobic core, one or more inorganic nanoparticles, and one or more lipids, incorporating at least one nucleic acid into the said lipid carrier to form a lipid carrier-nucleic acid complex, wherein the nucleic acid has a sequence comprising an antigen sequence as set forth in one of SEQ ID NOS: 1-2, 75, 76, 88, or 89; adding at least one cryoprotectant to the lipid carrier-nucleic acid complex to form a formulation, lyophilizing the formulation to form a lyophilized composition, and reconstituting the lyophilized composition in a suitable diluent.

Further provided herein are methods for reconstituting a spray-dried composition, wherein the methods comprise: obtaining a lipid carrier, wherein the lipid carrier is a nanoemulsion comprising a hydrophobic core, one or more inorganic nanoparticles, and one or more lipids, incorporating at least one nucleic acid into the said lipid carrier to form a lipid carrier-nucleic acid complex, wherein the nucleic acid has a sequence comprising an antigen sequence as set forth in one of SEQ ID NOS: 1-2, 75, 76, 88, or 89; adding at least one cryoprotectant to the lipid carrier-nucleic acid complex to form a formulation, spray drying the formulation to form a spray-dried composition, and reconstituting the spray-dried composition in a suitable diluent.

Additional features of the present invention will be apparent to persons of ordinary skill in the art in view of the following disclosure, as well as the accompanying drawings and claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1H show schematic representations of exemplary nanoparticle (NP) carriers. FIG. 1A shows an oil-in-water emulsion. FIG. 1B shows a nanostructured lipid carrier (NLC). FIG. 1C shows a nanoparticle having an inorganic nanoparticle in liquid oil. FIG. 1D shows a nanoparticle having a cationic lipid membrane and a liquid oil core. FIG. 1E shows an oil-in-water emulsion with two or more RNA or DNA molecules. FIG. 1F shows a nanostructured lipid carrier (NLC) with two or more RNA or DNA molecules. FIG. 1G shows a nanoparticle having an inorganic nanoparticle in liquid oil two or more RNA or DNA molecules. FIG. 1H shows a nanoparticle having a cationic lipid membrane, a liquid oil core, and two or more RNA or DNA molecules. Drawings not to scale.

FIGS. 2A-2B show the increased protein production induced by the Miglyol lipid carrier formulation, NP-3. FIG. 2A shows the first assay. FIG. 2B shows the second assay.

FIGS. 3A-3B show the decreased immune response induced by the Miglyol lipid carrier formulation, NP-3. FIG. 3A shows the first assay. FIG. 3B shows the second assay.

FIGS. 4A-4B show the correlation between enhanced protein production and low TNF (e.g., TNF alpha) stimulation observed with NP-3 as a result of the first and second assays. FIG. 4A shows the first assay. FIG. 4B shows the second assay.

FIGS. 5A-5F show SEAP levels in BALB/c mice injected intramuscularly with various embodiments of lipid carrier formulations described herein. FIG. 5A shows SEAP levels on day 4 post-injection. FIG. 5B shows SEAP levels on day 6 post-injection. FIG. 5C shows SEAP levels on day 8 post-injection. FIG. 5D shows SEAP levels on day 4 post-injection. FIG. 5E shows SEAP levels on day 6 post-injection. FIG. 5F shows SEAP levels on day 8 post-injection. X-axis: Condition, Y-axis: Relative light units (RLU).

FIG. 6 is a bar chart with measurements of Z-average measurement and polydispersity index (PDI) on the Y-axis and group number on the X-axis for conditions 1 to 14.

FIGS. 7A-7F show the effect of lipid carrier+TRP-1 replicon vaccine compositions on tumor volume in a B16 tumor model of melanoma. FIG. 7A shows untreated B16 mouse tumor volume over time. X-axis: time, Y-axis: tumor volume (mm³). FIG. 7B shows B16 mouse tumor volume over time for mice treated with lipid carrier+0.2 micrograms (μg) TRP-1 repRNA, at day 0 and day 14. X-axis: time, Y-axis: tumor volume (mm³). FIG. 7C shows B16 mouse tumor volume over time for mice treated with lipid carrier+0.2 μg TRP-1 repRNA, at day 0 only. X-axis: time, Y-axis: tumor volume (mm³). FIG. 7D shows B16 mouse tumor volume over time for mice treated with lipid carrier+1 μg TRP-1 repRNA, at day 0 and day 14. X-axis: time, Y-axis: tumor volume (mm³). FIG. 7E shows B16 mouse tumor volume over time for mice treated with lipid carrier+1 μg TRP-1 repRNA, at day 0 only. X-axis: time, Y-axis: tumor volume (mm³).

FIG. 7F shows animal survival rate over time. X axis: days after inoculation, Y-axis: percentage of animals alive.

FIG. 8 shows mean B16 tumor volume over time in untreated and lipid carrier+repRNA-TRP-1 vaccinated mice. X-axis: days post-implantation, Y-axis: tumor volume (mm³).

FIG. 9 shows individual tumor growth for control (scramble RNA) and animals that were prophylactically administered lipid carrier+1 μg TRP-1 repRNA vaccines prior to melanoma cell injection.

FIGS. 10A-10D show that immunization with MAGE-expressing replicon induces antigen-specific T cells. FIG. 10A shows CD8 T cells expressing Tbet and IFNγ. FIG. 10B shows the % IFNγ positive CD8 T cells in MAGE treated animals. FIG. 10C shows CD4 T cells producing IFNγ. FIG. 10D shows CD4 T cells producing IL-2. Flow cytometry plots are representative, while plots show data obtained from individual animals as well as mean and SEM.

FIGS. 11A-11B show graphs of mean tumor volume and survival of animals immunized with replicons encoding for MAGE-A3 (SEQ ID NO: 87) and TRP1 encoding for (SEQ ID NO: 78). FIG. 11A shows mean tumor volume (Y-axis) as a function of days post-implantation of tumors (X-axis). FIG. 11B shows the probability of survival in animals treated with no RNA, TRP-1/MAGE RNAs pre-palpable, and TRP-1/MAGE post-palpable. X-axis: Probability of survival; Y-axis: Days post-transplantation of tumors.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### DETAILED DESCRIPTION OF THE INVENTION

Provided herein are compositions, kits, methods, and uses thereof for inducing an immune response to a cancer cell or a tumor. Briefly, further described herein are (1) nucleic acids encoding for cancer-associated proteins, neoantigens, antibodies, and RNA polymerases; (2) nanoparticle carrier systems; (3) combination compositions; (4) thermally stable, dried, and lyophilized cancer vaccines; (5) pharmaceutical compositions; (6) dosing; (7) administration; (8) therapeutic applications; and (9) kits.

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document. All references disclosed herein, including patent references and non-patent references, are hereby incorporated by reference in their entirety as if each was incorporated individually. However, where a patent, patent application, or publication containing express definitions is incorporated by reference, those express definitions should be understood to apply to the incorporated patent, patent application, or publication in which they are found, and not necessarily to the text of this application, in particular the claims of this application, in which instance, the definitions provided herein are meant to supersede.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

Unless specifically stated or apparent from context, as used herein, the term “about” in reference to a number or range of numbers is understood to mean the stated number and numbers+/−20% thereof, or 20% below the lower listed limit and 20% above the higher listed limit for the values listed for a range.

The term “effective amount” or “therapeutically effective amount” refers to an amount that is sufficient to achieve or at least partially achieve the desired effect.

### Nucleic Acids

Provided herein are compositions comprising nucleic acids. In some embodiments, compositions provided herein comprise one or more types of nucleic acid sequences. In some embodiments, compositions provided herein comprise two or more types of nucleic acid sequences. In some embodiments, compositions provided herein comprise at least one DNA molecule. In some embodiments, compositions provided herein comprise at least one RNA molecule. In some embodiments, compositions provided herein comprise at least one DNA molecule and at least one RNA molecule.

In some embodiments, a nucleic acid provided herein is in complex with a nanoparticle provided herein. In some embodiments, the nucleic acid is in complex with a surface of the nanoparticle. In some embodiments, the nucleic acid is in complex with a hydrophilic surface of the nanoparticle. In some embodiments, the nucleic acid is located within the nanoparticle. In some embodiments, the nucleic acid is located within the hydrophobic core of the nanoparticle. In some embodiments, the nanoparticle is a lipid carrier nanoparticle, and the surface may be referred to herein as a membrane.

In some embodiments, nucleic acids provided herein comprise a deoxyribonucleic acid (DNA), a ribonucleic acid (RNA), a peptide nucleic acid (PNA), or a combination thereof. A nucleic acid can be linear or include a secondary structure (e.g., a hair pin). In some embodiments, the nucleic acid is a polynucleotide comprising modified nucleotides or bases, and/or their analogs. A polynucleotide may comprise modified nucleotides, such as methylated nucleotides and their analogs. If present, modification to the nucleotide structure may be imparted before or after assembly of compositions provided herein. Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); m1I (1-methylinosine); m′Im (1,2′-O-dimethylinosine); m3C (3-methylcytidine); Cm (2T-O-methylcytidine); s2C (2-thiocytidine); ac4C (N4-acetylcytidine); f5C (5-fonnylcytidine); m5Cm (5,2-O-dimethylcytidine); ac4Cm (N4acetyl2TOmethylcytidine); k2C (lysidine); mlG (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG(methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); hoSU (5-hydroxyuridine); moSU (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethylaminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-0-dimethyladenosine); m62Am (N6,N6,0-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Any one or any combination of these modified nucleobases may be included in the self-replicating RNA of the invention. Many of these modified nucleobases and their corresponding ribonucleosides are available from commercial suppliers. If desired, the nucleic acid can contain phosphoramidate, phosphorothioate, and/or methylphosphonate linkages. The RNA sequence can be modified with respect to its codon usage, for example, to increase translation efficacy and half-life of the RNA. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methyltransferase, which catalyzes the construction of N7-monomethylated cap 0 structures). Cap structure can provide stability and translational efficacy to the RNA molecule. The 5′ cap of the RNA molecule may be further modified by a 2′-O-Methyltransferase which results in the generation of a cap 1 structure (m7Gppp [m2′-O]N), which may further increase translation efficacy. A cap 1 structure may also increase in vivo potency.

In some embodiments, nucleic acids provided herein are present in an amount of about 5 ng to about 1 mg. In some embodiments, nucleic acids provided herein are present in an amount of up to about 25, 50, 75, 100, 150, 175 ng. In some embodiments, nucleic acids provided herein are present in an amount of up to about 1 mg. In some embodiments, nucleic acids provided herein are present in an amount of about 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, nucleic acids provided herein are present in an amount of 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, the nucleic acid is at least about 200, 250, 500, 750, 1000, 2000, 3000, 4000, 5000, 6000, 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is up to about 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is about 7500, 10,000, 15,000, or 20,000 nucleotides in length.

### Nucleic Acids Encoding Cancer-Associated Proteins

Provided herein are compositions, wherein the compositions comprise: a lipid carrier provided herein; and one or more nucleic acids, wherein the one or more nucleic acids comprises a sequence encoding for an antigen. In some embodiments, the antigen is a cancer-associated protein (also referred to as a tumor protein antigen or tumor antigen). In some embodiments, nucleic acids provided herein encode for a cancer-associated protein. In some embodiments, the cancer-associated protein is a surface protein, a cytosolic protein, or a transmembrane protein. In some embodiments, the cancer-associated protein is a protein that is expressed by a cancer cell. In some embodiments, the cancer-associated protein is a protein that is expressed by a microbial organism that causes a cancer (e.g., viral proteins).

In some embodiments, nucleic acids provided herein encode for a protein expressed by a solid cancer cell or a blood cancer cell. In some embodiments, the solid cancer cell is a melanoma cell. In some embodiments, the protein expressed by the melanoma cell is not expressed by a non-cancer cell. In some embodiments, the protein expressed by a melanoma cell comprises a mutation in the amino acid sequence relative to a comparable amino acid sequence in a non-cancer cell. In some embodiments, nucleic acids provided herein encode for MAGE-A1 (SEQ ID NO: 1) or a functional fragment thereof. In some embodiments, nucleic acids provided herein encode for MAGE-A3 (SEQ ID NO: 87) or a functional fragment thereof. In some embodiments, nucleic acids provided herein encode for TRP-1 (SEQ ID NO: 2) or a functional fragment thereof. In some embodiments, nucleic acids provided herein encode for TRP-1 and MAGE-A1. In some embodiments, nucleic acids provided herein encode for TRP-1 and MAGE-A3. In some embodiments, nucleic acids provided herein encode for a tyrosinase. In some embodiments, nucleic acids provided herein comprise a sequence that is at least 80% identical to SEQ ID NOS: 1, 2, 75, 76, 88, or 89. In some embodiments, nucleic acids provided herein comprise a sequence that is at least 80% identical to SEQ ID NOS: 1, 2, 75, 76, 88, or 89. In some embodiments, nucleic acids provided herein encode for an amino acid sequence listed in Table 1. In some embodiments, nucleic acids provided herein encode for an amino acid sequence that is at least 80% identical to SEQ ID NO: 77 or SEQ ID NO: 78. In some embodiments, nucleic acids provided herein encode for an amino acid sequence that is at least 80% identical to SEQ ID NO: 87. In some embodiments, compositions provided herein comprise two or more, three or more, four or more, five or more, six or more, or up to seven or more nucleic acids coding different sequences listed in Table 1. In some embodiments, nucleic acids provided herein encoding for a protein sequence listed in Table 1 is used as part of a treatment or prevention of melanoma. In some embodiments, a nucleic acid provided herein encodes for a cancer-associated protein listed in Table 1. In some embodiments, compositions provided herein comprise two or more nucleic acids encoding for different sequences listed in Table 1. In some embodiments, nucleic acids provided herein encode for a cancer-associated protein sequence comprising at least 80%, 85%, 90%, 95%, 96%, 97%, 98%, or 99% sequence identity to a sequence listed in Table 1. In some embodiments, compositions provided herein comprise two or more nucleic acids encoding different sequences listed in Table 1. In some embodiments, the nucleic acid provided herein encodes for a cancer-associated protein or a functional fragment thereof comprising at least 80%, 85%, 90%, 95%, 96%, 97%, 98%, or 99% sequence similarity to a sequence listed Table 1. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated.

TABLE 1
Exemplary Cancer-Associated Proteins on Melanoma Cells.

| | | Amino Acid |
|---|---|---|
| SEQ ID NO: | Reference Protein | Sequence |
| SEQ ID NO: 3 | Tyrosinase | KCDICTDEY |
| SEQ ID NO: 4 | Tyrosinase | YMDGTMSQV |
| SEQ ID NO: 5 | Tyrosinase | MLLAYLYQL |
| SEQ ID NO: 6 | Tyrosinase | AFLPWHRLF, |
| SEQ ID NO: 7 | Tyrosinase | SEIWRDIDF |
| SEQ ID NO: 8 | gp100/pMEL17 | YLEPGPVTA |
| SEQ ID NO: 9 | gp100/pMEL17 | KTWGQUWQV |
| SEQ ID NO: 10 | gp100/pMEL17 | ITDQVPFSV |
| SEQ ID NO: 11 | gp100/pMEL17 | VLYRYGSFSV |
| SEQ ID NO: 12 | gp100/pMEL17 | LLDGTATLRL |
| SEQ ID NO: 13 | gp100/pMEL17 | ALLAVGATK |
| SEQ ID NO: 14 | gp100/pMEL17 | MLGTHTMEV |
| SEQ ID NO: 15 | gp100/pMEL17 | LIYRRRLMK |
| SEQ ID NO: 16 | gp100/pMEL17 | ALNFPGSQK |
| SEQ ID NO: 17 | MART-1/MelanA | AAGIGILTV^{†} |
| SEQ ID NO: 18 | MART-1/MelanA | ILTVILGVL |
| SEQ ID NO: 19 | gp75/TRP-1 | MSLQRQFLR |
| SEQ ID NO: 20 | TRP-2 | SVYDFFVWL |
| SEQ ID NO: 21 | TRP-2 | LLGPGRPYR |
| SEQ ID NO: 22 | CEA | YLSGANLNL |
| SEQ ID NO: 23 | HER-2/neu | KIFGSLAFL |
| SEQ ID NO: 24 | HER-2/neu | VMAGVGSPYV |
| SEQ ID NO: 25 | HER-2/neu | IISAVVGIL |
| SEQ ID NO: 26 | PSMA | LLHETDSAV |
| SEQ ID NO: 27 | PSMA | ALFDIESKV |
| SEQ ID NO: 28 | MAGE-1 | EADPTGHSY |
| SEQ ID NO: 29 | MAGE-1 | SLFRAVITK |
| SEQ ID NO: 30 | MAGE-1 | SAYGEPRKL |
| SEQ ID NO: 31 | MAGE-2 | KMVELVHFL |
| SEQ ID NO: 32 | MAGE-2 | YLOLVFGIEV |
| SEQ ID NO: 33 | MAGE-3 | EVDPIGHLY |
| SEQ ID NO: 34 | MAGE-3 | FLWGPRALV |
| SEQ ID NO: 35 | MAGE-3 | MEVDPIGHLY |
| SEQ ID NO: 36 | BAGE | AARAVFLAL |
| SEQ ID NO: 37 | GAGE-1,2 | YRPRPRRY |
| SEQ ID NO: 38 | GnT-V | VLPDVFIRC |
| SEQ ID NO: 39 | NY-ESO-1 | QLSLLMWIT |
| SEQ ID NO: 40 | NY-ESO-1 | SLLMWITQC |
| SEQ ID NO: 41 | NY-ESO-1 | ASGPGGGAPR |
| SEQ ID NO: 42 | 43kD protein | QDLTMKYQIF |
| SEQ ID NO: 43 | p15 | (E)AYGLDFYIL |
| SEQ ID NO: 44 | Mutated beta-catenin | SYLDSGIHF^{‡} |
| SEQ ID NO: 45 | Mutated elongation factor 2 | ETVSEQSNV^{§} |
| SEQ ID NO: 46 | Mutated CASP-8 | FPSDSWCYF |
| | (FLICE/MACH) | |
| SEQ ID NO: 47 | MUM-1 gene product mutated | EEKLIVVLFT^{¶} |
| | across intron/exon junction | |
| SEQ ID NO: 77 | MAGE-A1 | See Sequences Section |
| SEQ ID NO: 78 | TRP-1 (also known as 5,6-dihydroxyindole-2- | See Sequences Section |
| | carboxylic acid oxidase, DHICA oxidase, | |
| | Melanoma antigen glycoprotein 75, Tyrosinase- | |
| | related protein 1, and TRYP-1) | |
| SEQ ID NO: 87 | MAGE-A3-melanoma-associated antigen 3 | See Sequences Section |
| SEQ ID NO: 90 | Prostein | See Sequences Section |

^{†}AAGIGILTV (SEQ ID NO: 91) is also recognized by HLA B45-1-restricted cytotoxic T lymphocyte.
^{‡}Phenylalanine (F) at position 9 is the result of mutation. The wild-type sequence is SYLDSGIHS (SEQ ID NO: 92).
^{§}Glutamine (Q) at position 6 is the result of somatic mutation. The wild-type sequence is ETVSEESNV (SEQ ID NO: 93).
^{¶}Isoleucine (I) at position 5 is the result of mutation. The wild-type sequence is EEKLSVVLF (SEQ ID NO: 94).

In some embodiments, a cancer-associated protein encoded by a nucleic acid provided herein comprises a cell membrane-contacting domain or functional fragment thereof. In some embodiments, the cell membrane-contacting domain comprises a transmembrane-binding domain, an outer cell membrane-contacting domain, or an inner cell membrane-contacting domain. In some embodiments, the cell membrane-contacting domain comprises a transmembrane-binding domain, an outer cell membrane-contacting domain, and an inner cell membrane-contacting domain. In some embodiments, the cancer-associated protein is a protein expressed by a melanoma cancer cell, a prostate cancer cell, a colon cancer cell, an ovarian cancer cell, a breast cancer cell, a pancreatic cancer cell, or a blood cell.

In some embodiments, a nucleic acid provided herein comprises a sequence encoding a dimer, trimer, or multimer of a cancer-associated protein provided herein. In some embodiments, nucleic acid provided herein comprises a sequence encoding an amino acid sequence that is at least about 500 amino acids in length or more. In some embodiments, nucleic acid provided herein comprises a sequence encoding an amino acid sequence that is at least about 200, 300, 400, 500, 750, 1000 or more amino acids in length or more. In some embodiments, nucleic acid provided herein comprises a sequence encoding one or more cancer-associated protein, wherein the one or more cancer-associated protein comprises a molecular weight of at least about 50 kiloDaltons (kDa) or more, at least about 100 kiloDaltons (kDa) or more, at least about 150 kiloDaltons (kDa) or more, at least about 200 kiloDaltons (kDa) or more, at least about 250 kiloDaltons (kDa) or more, at least about 300 kiloDaltons (kDa) or more, at least about 350 kiloDaltons (kDa) or more, at least about 400 kiloDaltons (kDa) or more, at least about 450 kiloDaltons (kDa) or more, at least about 500 kiloDaltons (kDa) or more, up to 1000 kDa or more. In some embodiments, nucleic acids provided herein comprise a sequence encoding one or more cancer-associated protein, wherein the one or more cancer-associated protein comprises a molecular weight of at least about 59 kDa or more.

In some embodiments, nucleic acids provided herein comprise a sequence encoding a cancer-associated protein associated with prostate cancer. In some embodiments, the cancer-associated protein with prostate cancer comprises prostein. In some embodiments, the cancer-associated protein is prostein. In some embodiments, the cancer-associated protein is at least about 50%, 60%, 70%, 80%, 90%, 95%, or 100% of full length prostein. In some embodiments, the prostein is human prostein. In some embodiments, the cancer-associated protein comprises an amino acid sequence that is at least about 80%, 85%, 90%, 95% or 100% identical to SEQ ID NO: 90.

### Cancer Antigen Binding Molecules

Provided herein are compositions, wherein the composition comprises: a lipid carrier provided herein; and a nucleic acid encoding for an antibody or a functional fragment thereof. In some embodiments, nucleic acids provided herein encode for a monoclonal antibody. In some embodiments, nucleic acids provided herein encode for a murine antibody, a humanized antibody, or a fully human antibody. In some embodiments, the antibody is an immunoglobulin (Ig) molecule. In some embodiments, the immunoglobulin molecule is an IgG, IgE, IgM, IgD, IgA, or an IgY isotype immunoglobulin molecule. Further provided herein are compositions, wherein the immunoglobulin molecule is an IgG1, an IgG2, an IgG3, an IgG4, an IgGA1, or an IgGA2 subclass immunoglobulin molecule. In some embodiments, the antibody is a recombinant antibody, a chimeric antibody, or a multivalent antibody. In some embodiments, the multivalent antibody is a bispecific antibody, a trispecific antibody, or a multispecific antibody. In some embodiments, the antibody or functional fragment is an antigen-binding fragment (Fab), and Fab2a F(ab′), a F(ab′)2, an dAb, an Fe, a Fv, a disulfide linked Fv, a scFv, a tandem scFv, a free LC, a half antibody, a single domain antibody (dAb), a diabody, or a nanobody.

In some embodiments, the antibody or functional fragment thereof specifically binds to a cancer-associated protein. In some embodiments, the antibody or functional fragment thereof is a cancer therapeutic antibody. In some embodiments, the cancer therapeutic antibody is atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab. Amino acid sequences for cancer therapeutic antibodies are provided below in Table 2.

In some embodiments, a nucleic acid provided herein encodes for a cancer-associated protein or an antibody amino acid sequence or a functional fragment thereof listed in Table 2. In some embodiments, compositions provided herein comprise two or more nucleic acids encoding for different sequences listed in Table 2. In some embodiments, nucleic acids provided herein encode for an antibody amino acid sequence comprising at least 80%, 85%, 90%, 95%, 96%, 97%, 98%, or 99% sequence identity to a sequence listed in Table 2. In some embodiments, compositions provided herein comprise two or more nucleic acids encoding different sequences listed in Table 2. In some embodiments, the nucleic acid provided herein encodes for an antibody or a functional fragment thereof comprising at least 80%, 85%, 90%, 95%, 96%, 97%, 98%, or 99% sequence similarity to a sequence listed Table 2.

TABLE 2
Cancer therapeutic antibodies.

| SEQ | Antibody Name | | |
|---|---|---|---|
| ID | (Commercial | Tumor | |
| NO: | Name) | Antigen | Heavy Chain Amino Acid Sequences |
| 48 | atezolizumab | CD274 | EVQLVESGGGLVQPGGSLRLSCAASGFTFSDSWIHWVRQAPGK |
| | (TECENTRIQ®) | (programmed | GLEWVAWISPYGGSTYYADSVKGRFTISADTSKNTAYLQMNSL |
| | | cell death 1 | RAEDTAVYYCARRHWPGGFDYWGQGTLVTVSSASTKGPSVFPL |
| | | ligand 1, B7H1, | APSSKSTSGGTAALGCLVKDYFPEPVTVSWNSGALTSGVHTFP |
| | | B7-H1, PDL1, | AVLQSSGLYSLSSVVTVPSSSLGTQTYICNVNHKPSNTKVDKK |
| | | PD-L1, | VEPKSCDKTHTCPPCPAPELLGGPSVFLFPPKPKDTLMISRTP |
| | | PDCD1L1, B7 | EVTCVVVDVSHEDPEVKFNWYVDGVEVHNAKTKPREEQYASTY |
| | | homolog 1, B7 | RVVSVLTVLHQDWLNGKEYKCKVSNKALPAPIEKTISKAKGQP |
| | | homologue 1) | REPQVYTLPPSREEMTKNQVSLTCLVKGFYPSDIAVEWESNGQ |
| | | [Homo sapiens] | PENNYKTTPPVLDSDGSFFLYSKLTVDKSRWQQGNVFSCSVMH |
| | | | EALHNHYTQKSLSLSPGK |
| 49 | avelumab | CD274 | EVQLLESGGGLVQPGGSLRLSCAASGFTFSSYIMMWVRQAPGK |
| | (BAVENCIO®) | | GLEWVSSIYPSGGITFYADTVKGRFTISRDNSKNTLYLQMNSL |
| | | | RAEDTAVYYCARIKLGTVTTVDYWGQGTLVTVSSASTKGPSVF |
| | | | PLAPSSKSTSGGTAALGCLVKDYFPEPVTVSWNSGALTSGVHT |
| | | | FPAVLQSSGLYSLSSVVTVPSSSLGTQTYICNVNHKPSNTKVD |
| | | | KKVEPKSCDKTHTCPPCPAPELLGGPSVFLFPPKPKDTLMISR |
| | | | TPEVTCVVVDVSHEDPEVKFNWYVDGVEVHNAKTKPREEQYNS |
| | | | TYRVVSVLTVLHQDWLNGKEYKCKVSNKALPAPIEKTISKAKG |
| | | | QPREPQVYTLPPSRDELTKNQVSLTCLVKGFYPSDIAVEWESN |
| | | | GQPENNYKTTPPVLDSDGSFFLYSKLTVDKSRWQQGNVFSCSV |
| | | | MHEALHNHYTQKSLSLSPGK |
| 50 | bevacizumab | VEGFA | EVQLVESGGGLVQPGGSLRLSCAASGYTFTNYGMNWVRQAPGK |
| | (AVASTIN®) | (vascular | GLEWVGWINTYTGEPTYAADFKRRFTFSLDTSKSTAYLQMNSL |
| | | endothelial | RAEDTAVYYCAKYPHYYGSSHWYFDVWGQGTLVTVSSASTKGP |
| | | growth factor | SVFPLAPSSKSTSGGTAALGCLVKDYFPEPVTVSWNSGALTSG |
| | | A, VEGF-A, | VHTFPAVLQSSGLYSLSSVVTVPSSSLGTQTYICNVNHKPSNT |
| | | VEGF) [Homo | KVDKKVEPKSCDKTHTCPPCPAPELLGGPSVFLFPPKPKDTLM |
| | | sapiens] | ISRTPEVTCVVVDVSHEDPEVKFNWYVDGVEVHNAKTKPREEQ |
| | | | YNSTYRVVSVLTVLHQDWLNGKEYKCKVSNKALPAPIEKTISK |
| | | | AKGQPREPQVYTLPPSREEMTKNQVSLTCLVKGFYPSDIAVEW |
| | | | ESNGQPENNYKTTPPVLDSDGSFFLYSKLTVDKSRWQQGNVFS |
| | | | CSVMHEALHNHYTQKSLSLSPGK |
| 51 | cemiplimab | PDCD1 | EVQLLESGGVLVQPGGSLRLSCAASGFTFSNFGMTWVRQAPGK |
| | (LIBTAYO®) | (programmed | GLEWVSGISGGGRDTYFADSVKGRFTISRDNSKNTLYLQMNSL |
| | | cell death 1, | KGEDTAVYYCVKWGNIYFDYWGQGTLVTVSSASTKGPSVFPLA |
| | | PD1, PD-1, | PCSRSTSESTAALGCLVKDYFPEPVTVSWNSGALTSGVHTFPA |
| | | CD279) [Homo | VLQSSGLYSLSSVVTVPSSSLGTKTYTCNVDHKPSNTKVDKRV |
| | | sapiens] | ESKYGPPCPPCPAPEFLGGPSVFLFPPKPKDTLMISRTPEVTC |
| | | | VVVDVSQEDPEVQFNWYVDGVEVHNAKTKPREEQFNSTYRVVS |
| | | | VLTVLHQDWLNGKEYKCKVSNKGLPSSIEKTISKAKGQPREPQ |
| | | | VYTLPPSQEEMTKNQVSLTCLVKGFYPSDIAVEWESNGQPENN |
| | | | YKTTPPVLDSDGSFFLYSRLTVDKSRWQEGNVFSCSVMHEALH |
| | | | NHYTQKSLSLSLGK |
| 52 | cetuximab | EGFR | QVQLKQSGPGLVQPSQSLSITCTVSGFSLTNYGVHWVRQSPGK |
| | (ERBITUX®) | (epidermal | GLEWLGVIWSGGNTDYNTPFTSRLSINKDNSKSQVFFKMNSLQ |
| | | growth factor | SNDTAIYYCARALTYYDYEFAYWGQGTLVTVSAASTKGPSVFP |
| | | receptor, | LAPSSKSTSGGTAALGCLVKDYFPEPVTVSWNSGALTSGVHTF |
| | | receptor | PAVLQSSGLYSLSSVVTVPSSSLGTQTYICNVNHKPSNTKVDK |
| | | tyrosine-protein | RVEPKSCDKTHTCPPCPAPELLGGPSVFLFPPKPKDTLMISRT |
| | | kinase erbB-1, | PEVTCVVVDVSHEDPEVKFNWYVDGVEVHNAKTKPREEQYNST |
| | | ERBB1, HER1, | YRVVSVLTVLHQDWLNGKEYKCKVSNKALPAPIEKTISKAKGQ |
| | | HER-1, ERBB) | PREPQVYTLPPSREEMTKNQVSLTCLVKGFYPSDIAVEWESNG |
| | | [Homo sapiens] | QPENNYKTTPPVLDSDGSFFLYSKLTVDKSRWQQGNVFSCSVM |
| | | | HEALHNHYTQKSLSLSPGK |
| 53 | daratumumab | CD38 (ADP- | EVQLLESGGGLVQPGGSLRLSCAVSGFTFNSFAMSWVRQAPGK |
| | (DARZALEX™; | ribosyl cyclase | GLEWVSAISGSGGGTYYADSVKGRFTISRDNSKNTLYLQMNSL |
| | DARZALEX; | 1, cyclic ADP- | RAEDTAVYFCAKDKILWFGEPVFDYWGQGTLVTVSSASTKGPS |
| | FASPRO™) | ribose | VFPLAPSSKSTSGGTAALGCLVKDYFPEPVTVSWNSGALTSGV |
| | | hydrolase 1, | HTFPAVLQSSGLYSLSSVVTVPSSSLGTQTYICNVNHKPSNTK |
| | | cADPr | VDKRVEPKSCDKTHTCPPCPAPELLGGPSVFLFPPKPKDTLMI |
| | | hydrolase 1, | SRTPEVTCVVVDVSHEDPEVKFNWYVDGVEVHNAKTKPREEQY |
| | | T10) [Homo | NSTYRVVSVLTVLHQDWLNGKEYKCKVSNKALPAPIEKTISKA |
| | | sapiens] | KGQPREPQVYTLPPSREEMTKNQVSLTCLVKGFYPSDIAVEWE |
| | | | SNGQPENNYKTTPPVLDSDGSFFLYSKLTVDKSRWQQGNVFSC |
| | | | SVMHEALHNHYTQKSLSLSPGK |
| 54 | dinutuximab | ganglioside | EVQLLQSGPELEKPGASVMISCKASGSSFTGYNMNWVRQNIGK |
| | (UNITUXIN™) | GD2 | SLEWIGAIDPYYGGTSYNQKFKGRATLTVDKSSSTAYMHLKSL |
| | | (disialoganglio- | TSEDSAVYYCVSGMEYWGQGTSVTVSSASTKGPSVFPLAPSSK |
| | | side GD2) | STSGGTAALGCLVKDYFPEPVTVSWNSGALTSGVHTFPAVLQS |
| | | [Homo sapiens] | SGLYSLSSVVTVPSSSLGTQTYICNVNHKPSNTKVDKRVEPKS |
| | | | CDKTHTCPPCPAPELLGGPSVFLFPPKPKDTLMISRTPEVTCV |
| | | | VVDVSHEDPEVKFNWYVDGVEVHNAKTKPREEQYNSTYRVVSV |
| | | | LTVLHQDWLNGKEYKCKVSNKALPAPIEKTISKAKGQPREPQV |
| | | | YTLPPSREEMTKNQVSLTCLVKGFYPSDIAVEWESNGQPENNY |
| | | | KTTPPVLDSDGSFFLYSKLTVDKSRWQQGNVFSCSVMHEALHN |
| | | | HYTQKSLSLSPGK |
| 55 | durvalumab | CD274 | EVQLVESGGGLVQPGGSLRLSCAASGFTFSRYWMSWVRQAPGK |
| | (IMFINZI™) | | GLEWVANIKQDGSEKYYVDSVKGRFTISRDNAKNSLYLQMNSL |
| | | | RAEDTAVYYCAREGGWFGELAFDYWGQGTLVTVSSASTKGPSV |
| | | | FPLAPSSKSTSGGTAALGCLVKDYFPEPVTVSWNSGALTSGVH |
| | | | TFPAVLQSSGLYSLSSVVTVPSSSLGTQTYICNVNHKPSNTKV |
| | | | DKRVEPKSCDKTHTCPPCPAPEFEGGPSVFLFPPKPKDTLMIS |
| | | | RTPEVTCVVVDVSHEDPEVKFNWYVDGVEVHNAKTKPREEQYN |
| | | | STYRVVSVLTVLHQDWLNGKEYKCKVSNKALPASIEKTISKAK |
| | | | GQPREPQVYTLPPSREEMTKNQVSLTCLVKGFYPSDIAVEWES |
| | | | NGQPENNYKTTPPVLDSDGSFFLYSKLTVDKSRWQQGNVFSCS |
| | | | VMHEALHNHYTQKSLSLSPGK |
| 56 | elotuzumab | SLAMF7 | EVQLVESGGGLVQPGGSLRLSCAASGFDFSRYWMSWVRQAPGK |
| | (EMPLICITI™) | (SLAM family | GLEWIGEINPDSSTINYAPSLKDKFIISRDNAKNSLYLQMNSL |
| | | member 7, CD2 | RAEDTAVYYCARPDGNYWYFDVWGQGTLVTVSSASTKGPSVFP |
| | | subset 1, CS1, | LAPSSKSTSGGTAALGCLVKDYFPEPVTVSWNSGALTSGVHTF |
| | | CD2-like | PAVLQSSGLYSLSSVVTVPSSSLGTQTYICNVNHKPSNTKVDK |
| | | receptor- | KVEPKSCDKTHTCPPCPAPELLGGPSVFLFPPKPKDTLMISRT |
| | | activating | PEVTCVVVDVSHEDPEVKFNWYVDGVEVHNAKTKPREEQYNST |
| | | cytotoxic cells, | YRVVSVLTVLHQDWLNGKEYKCKVSNKALPAPIEKTISKAKGQ |
| | | CRACC, | PREPQVYTLPPSRDELTKNQVSLTCLVKGFYPSDIAVEWESNG |
| | | 19A24, CD319) | QPENNYKTTPPVLDSDGSFFLYSKLTVDKSRWQQGNVFSCSVM |
| | | [Homo sapiens] | HEALHNHYTQKSLSLSPGK |
| 57 | ipilimumab | CTLA4 | QVQLVESGGGVVQPGRSLRLSCAASGFTFSSYTMHWVRQAPGK |
| | (YERVOY®) | (cytotoxic T- | GLEWVTFISYDGNNKYYADSVKGRFTISRDNSKNTLYLQMNSL |
| | | lymphocyte- | RAEDTAIYYCARTGWLGPFDYWGQGTLVTVSSASTKGPSVFPL |
| | | associated | APSSKSTSGGTAALGCLVKDYFPEPVTVSWNSGALTSGVHTFP |
| | | protein 4, | AVLQSSGLYSLSSVVTVPSSSLGTQTYICNVNHKPSNTKVDKR |
| | | CD152) [Homo | VEPKSCDKTHTCPPCPAPELLGGPSVFLFPPKPKDTLMISRTP |
| | | sapiens] | EVTCVVVDVSHEDPEVKFNWYVDGVEVHNAKTKPREEQYNSTY |
| | | | RVVSVLTVLHQDWLNGKEYKCKVSNKALPAPIEKTISKAKGQP |
| | | | REPQVYTLPPSRDELTKNQVSLTCLVKGFYPSDIAVEWESNGQ |
| | | | PENNYKTTPPVLDSDGSFFLYSKLTVDKSRWQQ |
| | | | GNVFSCSVMHEALHNHYTQKSLSLSPGK |
| 58 | isatuximab | CD38 (ADP- | QVQLVQSGAEVAKPGTSVKLSCKASGYTFTDYWMQWVKQRPGQ |
| | (SARCLISA®) | ribosyl cyclase | GLEWIGTIYPGDGDTGYAQKFQGKATLTADKSSKTVYMHLSSL |
| | | 1, cyclic ADP- | ASEDSAVYYCARGDYYGSNSLDYWGQGTSVTVSSASTKGPSVF |
| | | ribose | PLAPSSKSTSGGTAALGCLVKDYFPEPVTVSWNSGALTSGVHT |
| | | hydrolase 1, | FPAVLQSSGLYSLSSVVTVPSSSLGTQTYICNVNHKPSNTKVD |
| | | cADPr | KKVEPKSCDKTHTCPPCPAPELLGGPSVFLFPPKPKDTLMISR |
| | | hydrolase 1, | TPEVTCVVVDVSHEDPEVKFNWYVDGVEVHNAKTKPREEQYNS |
| | | T10) [Homo | TYRVVSVLTVLHQDWLNGKEYKCKVSNKALPAPIEKTISKAKG |
| | | sapiens] | QPREPQVYTLPPSRDELTKNQVSLTCLVKGFYPSDIAVEWESN |
| | | | GQPENNYKTTPPVLDSDGSFFLYSKLTVDKSRWQQGNVFSCSV |
| | | | MHEALHNHYTQKSLSLSPGK |
| 59 | mogamulizumab | CCR4 | EVQLVESGGDLVQPGRSLRLSCAASGFIFSNYGMSWVRQAPGK |
| | (PQTELIGEQ®) | (chemokine (C- | GLEWVATISSASTYSYYPDSVKGRFTISRDNAKNSLYLQMNSL |
| | | C motif) | RVEDTALYYCGRHSDGNFAFGYWGQGTLVTVSSASTKGPSVFP |
| | | receptor 4, CC | LAPSSKSTSGGTAALGCLVKDYFPEPVTVSWNSGALTSGVHTF |
| | | chemokine | PAVLQSSGLYSLSSVVTVPSSSLGTQTYICNVNHKPSNTKVDK |
| | | receptor 4, | KVEPKSCDKTHTCPPCPAPELLGGPSVFLFPPKPKDTLMISRT |
| | | CCR-4, CKR4, | PEVTCVVVDVSHEDPEVKFNWYVDGVEVHNAKTKPREEQYNST |
| | | k5-5, CD194) | YRVVSVLTVLHQDWLNGKEYKCKVSNKALPAPIEKTISKAKGQ |
| | | [Homo sapiens] | PREPQVYTLPPSRDELTKNQVSLTCLVKGFYPSDIAVEWESNG |
| | | | QPENNYKTTPPVLDSDGSFFLYSKLTVDKSRWQQGNVFSCSVM |
| | | | HEALHNHYTQKSLSLSPGK |
| 60 | necitumumab | EGFR | QVQLQESGPGLVKPSQTLSLTCTVSGGSISSGDYYWSWIRQPP |
| | (PQRTRAZZA™) | (epidermal | GKGLEWIGYIYYSGSTDYNPSLKSRVTMSVDTSKNQFSLKVNS |
| | | growth factor | VTAADTAVYYCARVSIFGVGTFDYWGQGTLVTVSSASTKGPSV |
| | | receptor, | LPLAPSSKSTSGGTAALGCLVKDYFPEPVTVSWNSGALTSGVH |
| | | receptor | TFPAVLQSSGLYSLSSVVTVPSSSLGTQTYICNVNHKPSNTKV |
| | | tyrosine-protein | DKRVEPKSCDKTHTCPPCPAPELLGGPSVFLFPPKPKDTLMIS |
| | | kinase erbB-1, | RTPEVTCVVVDVSHEDPEVKFNWYVDGVEVHNAKTKPREEQYN |
| | | ERBB1, HER1, | STYRVVSVLTVLHQDWLNGKEYKCKVSNKALPAPIEKTISKAK |
| | | HER-1, ERBB) | GQPREPQVYTLPPSREEMTKNQVSLTCLVKGFYPSDIAVEWES |
| | | [Homo sapiens] | NGQPENNYKTTPPVLDSDGSFFLYSKLTVDKSRWQQGNVFSCS |
| | | | VMHEALHNHYTQKSLSLSPGK |
| 61 | nivolumab | PDCD1 | QVQLVESGGGVVQPGRSLRLDCKASGITFSNSGMHWVRQAPGK |
| | (QPDIVO®) | (programmed | GLEWVAVIWYDGSKRYYADSVKGRFTISRDNSKNTLFLQMNSL |
| | | cell death 1, | RAEDTAVYYCATNDDYWGQGTLVTVSSASTKGPSVFPLAPCSR |
| | | PD1, PD-1, | STSESTAALGCLVKDYFPEPVTVSWNSGALTSGVHTFPAVLQS |
| | | CD279) [Homo | SGLYSLSSVVTVPSSSLGTKTYTCNVDHKPSNTKVDKRVESKY |
| | | sapiens] | GPPCPPCPAPEFLGGPSVFLFPPKPKDTLMISRTPEVTCVVVD |
| | | | VSQEDPEVQFNWYVDGVEVHNAKTKPREEQFNSTYRVVSVLTV |
| | | | LHQDWLNGKEYKCKVSNKGLPSSIEKTISKAKGQPREPQVYTL |
| | | | PPSQEEMTKNQVSLTCLVKGFYPSDIAVEWESNGQPENNYKTT |
| | | | PPVLDSDGSFFLYSRLTVDKSRWQEGNVFSCSVMHEALHNHYT |
| | | | QKSLSLSLGK |
| 62 | obinutuzumab | MS4A1 | QVQLVQSGAEVKKPGSSVKVSCKASGYAFSYSWINWVRQAPGQ |
| | (GAZYVA®) | (membrane- | GLEWMGRIFPGDGDTDYNGKFKGRVTITADKSTSTAYMELSSL |
| | | spanning 4- | RSEDTAVYYCARNVFDGYWLVYWGQGTLVTVSSASTKGPSVFP |
| | | domains | LAPSSKSTSGGTAALGCLVKDYFPEPVTVSWNSGALTSGVHTF |
| | | subfamily A | PAVLQSSGLYSLSSVVTVPSSSLGTQTYICNVNHKPSNTKVDK |
| | | member 1, | KVEPKSCDKTHTCPPCPAPELLGGPSVFLFPPKPKDTLMISRT |
| | | CD20) [Homo | PEVTCVVVDVSHEDPEVKFNWYVDGVEVHNAKTKPREEQYNST |
| | | sapiens] | YRVVSVLTVLHQDWLNGKEYKCKVSNKALPAPIEKTISKAKGQ |
| | | | PREPQVYTLPPSRDELTKNQVSLTCLVKGFYPSDIAVEWESNG |
| | | | QPENNYKTTPPVLDSDGSFFLYSKLTVDKSRWQQGNVFSCSVM |
| | | | HEALHNHYTQKSLSLSPGK |
| 63 | ofatumumab | MS4A1 | EVQLVESGGGLVQPGRSLRLSCAASGFTFNDYAMHWVRQAPGK |
| | (ARZERRA® | (membrane- | GLEWVSTISWNSGSIGYADSVKGRFTISRDNAKKSLYLQMNSL |
| | KESIMPTA®) | spanning 4- | RAEDTALYYCAKDIQYGNYYYGMDVWGQGTTVTVSSASTKGPS |
| | | domains | VFPLAPGSSKSTSGTAALGCLVKDYFPEPVTVSWNSGALTSGV |
| | | subfamily A | HTFPAVLQSSGLYSLSSVVTVPSSSLGTQTYICNVNHKPSNTK |
| | | member 1, | VDKKVEP |
| | | CD20) [Homo | |
| | | sapiens] | |
| 64 | olaratumab | PDGFRA | QLQLQESGPGLVKPSETLSLTCTVSGGSINSSSYYWGWLRQSP |
| | (LARTRUVO™) | (platelet- | GKGLEWIGSFFYTGSTYYNPSLRSRLTISVDTSKNQFSLMLSS |
| | | derived growth | VTAADTAVYYCARQSTYYYGSGNYYGWFDRWDQGTLVTVSSAS |
| | | factor receptor | TKGPSVFPLAPSSKSTSGGTAALGCLVKDYFPEPVTVSWNSGA |
| | | alpha subunit, | LTSGVHTFPAVLQSSGLYSLSSVVTVPSSSLGTQTYICNVNHK |
| | | PDGFR2, | PSNTKVDKRVEPKSCDKTHTCPPCPAPELLGGPSVFLFPPKPK |
| | | CD140a) | DTLMISRTPEVTCVVVDVSHEDPEVKFNWYVDGVEVHNAKTKP |
| | | [Homo sapiens] | REEQYNSTYRVVSVLTVLHQDWLNGKEYKCKVSNKALPAPIEK |
| | | | TISKAKGQPREPQVYTLPPSREEMTKNQVSLTCLVKGFYPSDI |
| | | | AVEWESNGQPENNYKTTPPVLDSDGSFFLYSKLTVDKSRWQQG |
| | | | NVFSCSVMHEALHNHYTQKSLSLSPG |
| 65 | panitumumab | EGFR | QVQLQESGPGLVKPSETLSLTCTVSGGSVSSGDYYWTWIRQSP |
| | (VECTIBIX®) | (epidermal | GKGLEWIGHIYYSGNTNYNPSLKSRLTISIDTSKTQFSLKLSS |
| | | growth factor | VTAADTAIYYCVRDRVTGAFDIWGQGTMVTVSSASTKGPSVFP |
| | | receptor, | LAPCSRSTSESTAALGCLVKDYFPEPVTVSWNSGALTSGVHTF |
| | | receptor | PAVLQSSGLYSLSSVVTVPSSNFGTQTYTCNVDHKPSNTKVDK |
| | | tyrosine-protein | TVERK |
| | | kinase erbB-1, | |
| | | ERBB1, HER1, | |
| | | HER-1, ERBB) | |
| | | [Homo sapiens] | |
| 66 | pembrolizumab | PDCD1 | VQLVQSGVEVKKPGASVKVSCKASGYTFTNYYMYWVRQAPGQG |
| | (KEYTRUDA®) | (programmed | LEWMGGINPSNGGTNFNEKFKNRVTLTTDSSTTTAYMELKSLQ |
| | | cell death 1, | FDDTAVYYCARRDYRFDMGFDYWGQGTTVTVSSASTKGPSVFP |
| | | PD1, PD-1, | LAPCSRSTSESTAALGCLVKDYFPEPVTVSWNSGALTSGVHTF |
| | | CD279) [Homo | PAVLQSSGLYSLSSVVTVPSSSLGTKTYTCNVDHKPSNTKVDK |
| | | sapiens] | RVESKYGPPCPPCPAPEFLGGPSVFLFPPKPKDTLMISRTPEV |
| | | | TCVVVDVSQEDPEVQFNWYVDGVEVHNAKTKPREEQFNSTYRV |
| | | | VSVLTVLHQDWLNGKEYKCKVSNKGLPSSIEKTISKAKGQPRE |
| | | | PQVYTLPPSQEEMTKNQVSLTCLVKGFYPSDIAVEWESNGQPE |
| | | | NNYKTTPPVLDSDGSFFLYSRLTVDKSRWQEGNVFSCSVMHEA |
| | | | LHNHYTQKSLSLSLGK |
| 67 | pertuzumab | ERBB2 | EVQLVESGGGLVQPGGSLRLSCAASGFTFTDYTMDWVRQAPGK |
| | (PERJETA®) | (epidermal | GLEWVADVNPNSGGSIYNQRFKGRFTLSVDRSKNTLYLQMNSL |
| | | growth factor | RAEDTAVYYCARNLGPSFYFDYWGQGTLVTVSSASTKGPSVFP |
| | | receptor 2, | LAPSSKSTSGGTAALGCLVKDYFPEPVTVSWNSGALTSGVHTF |
| | | receptor | PAVLQSSGLYSLSSVVTVPSSSLGTQTYICNVNHKPSNTKVDK |
| | | tyrosine-protein | KVEPKSCDKTH |
| | | kinase erbB-2, | |
| | | EGFR2, HER2, | |
| | | HER-2, p185c- | |
| | | erbB2, NEU, | |
| | | CD340) [Homo | |
| | | sapiens] | |
| 68 | ramucirumab | KDR (kinase | EVQLVQSGGGLVKPGGSLRLSCAASGFTFSSYSMNWVRQAPGK |
| | (CYRAMZA™) | insert domain | GLEWVSSISSSSSYIYYADSVKGRFTISRDNAKNSLYLQMNSL |
| | | receptor, | RAEDTAVYYCARVTDAFDIWGQGTMVTVSSASTKGPSVLPLAP |
| | | vascular | SSKSTSGGTAALGCLVKDYFPEPVTVSWNSGALTSGVHTFPAV |
| | | endothelial | LQSSGLYSLSSVVTVPSSSLGTQTYICNVNHKPSNTKVDKRVE |
| | | growth factor | PKSCDKTHTCPPCPAPELLGGPSVFLFPPKPKDTLMISRTPEV |
| | | receptor 2, | TCVVVDVSHEDPEVKFNWYVDGVEVHNAKTKPREEQYNSTYRV |
| | | VEGFR2, | VSVLTVLHQDWLNGKEYKCKVSNKALPAPIEKTISKAKGQPRE |
| | | VEGF-R2, | PQVYTLPPSREEMTKNQVSLTCLVKGFYPSDIAVEWESNGQPE |
| | | FLK1, CD309) | NNYKTTPPVLDSDGSFFLYSKLTVDKSRWQQGNVFSCSVMHEA |
| | | [Homo sapiens] | LHNHYTQKSLSLSPGK |
| 69 | rituximab | MS4A1 | QVQLQQPGAELVKPGASVKMSCKASGYTFTSYNMHWVKQTPGR |
| | (RITUXAN®) | (membrane- | GLEWIGAIYPGNGDTSYNQKFKGKATLTADKSSSTAYMQLSSL |
| | | spanning 4- | TSEDSAVYYCARSTYYGGDWYFNVWGAGTTVTVSAASTKGPSV |
| | | domains | FPLAPSSKSTSGGTAALGCLVKDYFPEPVTVSWNSGALTSGVH |
| | | subfamily A | TFPAVLQSSGLYSLSSVVTVPSSSLGTQTYICNVNHKPSNTKV |
| | | member 1, | DKKVEPKSCDKTHTCPPCPAPELLGGPSVFLFPPKPKDTLMIS |
| | | CD20) [Homo | RTPEVTCVVVDVSHEDPEVKFNWYVDGVEVHNAKTKPREEQYN |
| | | sapiens] | STYRVVSVLTVLHQDWLNGKEYKCKVSNKALPAPIEKTISKAK |
| | | | GQPREPQVYTLPPSRDELTKNQVSLTCLVKGFYPSDIAVEWES |
| | | | NGQPENNYKTTPPVLDSDGSFFLYSKLTVDKSRWQQGNVFSCS |
| | | | VMHEALHNHYTQKSLSLSPGK |
| 70 | trastuzumab | ERBB2 | EVQLVESGGGLVQPGGSLRLSCAASGFNIKDTYIHWVRQAPGK |
| | (HERCEPTIN®) | (epidermal | GLEWVARIYPTNGYTRYADSVKGRFTISADTSKNTAYLQMNSL |
| | | growth factor | RAEDTAVYYCSRWGGDGFYAMDYWGQGTLVTVSSASTKGPSVF |
| | | receptor 2, | PLAPSSKSTSGGTAALGCLVKDYFPEPVTVSWNSGALTSGVHT |
| | | receptor | FPAVLQSSGLYSLSSVVTVPSSSLGTQTYICNVNHKPSNTKVD |
| | | tyrosine-protein | KKVEPKSCDKTHTCPPCPAPELLGGPSVFLFPPKPKDTLMISR |
| | | kinase erbB-2, | TPEVTCVVVDVSHEDPEVKFNWYVDGVEVHNAKTKPREEQYNS |
| | | EGFR2, HER2, | TYRVVSVLTVLHQDWLNGKEYKCKVSNKALPAPIEKTISKAKG |
| | | HER-2, p185c- | QPREPQVYTLPPSREEMTKNQVSLTCLVKGFYPSDIAVEWESN |
| | | erbB2, NEU, | GQPENNYKTTPPVLDSDGSFFLYSKLTVDKSRWQQGNVFSCSV |
| | | CD340) [Homo | MHEALHNHYTQKSLSLSPG |
| | | sapiens] | |

*Sequences in Table 2 were determined by IMGT-monoclonal antibody database

As an alternative to, or in addition to the delivery of RNAs as antigens, combinations can be used, for example, RNA antigens combined with RNAs that stimulate innate immune responses, or RNAs that launch oncolytic viruses, or live-attenuated viruses, or agonists generally that stimulate immune responses including TLRs (toll-like receptors), RLR (RIG-I-like receptors), or NLRs (nod-like receptors). In certain embodiments, a composition provided herein comprises a combination of RNA-encoded antigens with another RNA that can stimulate innate immune responses or can launch oncolytic viruses or live-attenuated viruses. Alternatively, compositions provided herein that contain RNA-encoded antigens can be combined with a formulation that contains another RNA or other immune response agonist that can stimulate innate immune responses or can launch oncolytic viruses or live-attenuated viruses.

### Self-Replicating Nucleic Acids

Provided herein are compositions comprising a self-replicating nucleic acid. The cancer-associated antigen or cancer therapeutic antibody provided herein or fragment thereof can be encoded as part of a self-replicating nucleic acid construct. In some embodiments, the self-replicating nucleic acid molecule comprises at least one or more genes selected from the group consisting of viral replicases, viral proteases, viral helicases and other nonstructural viral proteins, and also comprises 5′- and 3′-end cis-active replication sequences, and an antigenic sequence encoding for a cancer-associated antigen. A subgenomic promoter that directs expression of the heterologous sequence(s) can be included in the self-replicating nucleotide sequence. If desired, a heterologous sequence may be fused in frame to other coding regions in the self-replicating RNA and/or may be under the control of an internal ribosome entry site (IRES).

In some embodiments, the self-replicating nucleotide sequence is a self-replicating RNA molecule. Self-replicating RNA molecules are designed so that the self-replicating RNA molecule cannot induce production of infectious viral particles. This can be achieved, for example, by omitting one or more viral genes encoding for structural proteins that are necessary for the production of viral particles in the self-replicating RNA. For example, when the self-replicating RNA molecule is based on an alphavirus, such as Sindbis virus (SIN), Semliki forest virus and Venezuelan equine encephalitis virus (VEE), one or more genes encoding for viral structural proteins, such as capsid and/or envelope glycoproteins, can be omitted. If desired, self-replicating RNA molecules of the invention can be designed to induce production of infectious viral particles that are attenuated or virulent, or to produce viral particles that are capable of a single round of subsequent infection.

A self-replicating RNA molecule can, when delivered to an animal cell even without any proteins, lead to the production of multiple daughter RNAs by transcription from itself (or from an antisense copy of itself). The self-replicating RNA can be directly translated after delivery to a cell, and this translation provides an RNA-dependent RNA polymerase which then produces transcripts from the delivered RNA. Thus, the delivered RNA leads to the production of multiple daughter RNAs. These transcripts are antisense relative to the delivered RNA and may be translated themselves to provide in situ expression of encoded cancer-associated antigen, or may be transcribed to provide further transcripts with the same sense as the delivered RNA which are translated to provide in situ expression of the encoded cancer-associated antigen(s).

The self-replicating RNA molecules provided herein can contain one or more modified nucleotides and therefore have improved stability and be resistant to degradation and clearance in vivo, and other advantages. In some embodiments, self-replicating RNA molecules that contain modified nucleotides avoid or reduce stimulation of endosomal and cytoplasmic immune receptors when the self-replicating RNA is delivered into a cell. This permits self-replication, amplification and expression of protein to occur. This also reduces safety concerns relative to self-replicating RNA that does not contain modified nucleotides, because the self-replicating RNA that contains modified nucleotides reduce activation of the innate immune system and subsequent undesired consequences (e.g., inflammation at injection site, irritation at injection site, pain, and the like). RNA molecules produced as a result of self-replication are recognized as foreign nucleic acids by the cytoplasmic immune receptors. Thus, self-replicating RNA molecules that contain modified nucleotides provide for efficient amplification of the RNA in a host cell and expression of cancer-associated antigen spike proteins, as well as adjuvant effects.

In some embodiments, self-replicating RNA molecules provided herein contain at least one modified nucleotide. Modified nucleotides that are not part of the 5′ cap (e.g., in addition to the modification that are part of the 5″ cap) can be used. Accordingly, the self-replicating RNA molecule can contain a modified nucleotide at a single position, can contain a particular modified nucleotide (e.g., pseudouridine, N6-methyladenosine, 5-methylcytidine, 5-methyluridine) at two or more positions, or can contain two, three, four, five, six, seven, eight, nine, ten or more modified nucleotides (e.g., each at one or more positions). Preferably, the self-replicating RNA molecules comprise modified nucleotides that contain a modification on or in the nitrogenous base, but do not contain modified sugar or phosphate moieties. In some examples, between 0.001% and 99% or 100% of the nucleotides in a self-replicating RNA molecule are modified nucleotides. For example, 0.001%-25%, 0.01%-25%, 0.1%-25%, or 1%-25% of thenucleotides in a self-replicating RNA molecule are modified nucleotides. In other examples, between 0.001% and 99% or 100% of a particular unmodified nucleotide in a self-replicating RNA molecule is replaced with a modified nucleotide. For example, about 1% of the nucleotides in the self-replicating RNA molecule that contain uridine can be modified, such as by replacement of uridine with pseudouridine. In other examples, the desired amount (percentage) of two, three, or four particular nucleotides (nucleotides that contain uridine, cytidine, guanosine, or adenine) in a self-replicating RNA molecule are modified nucleotides. For example, 0.001%-²⁵%, 0.01%-²⁵%, 0.1%-²⁵%, or 1%-²⁵% of a particular nucleotide in a self-replicating RNA molecule are modified nucleotides. In other examples, 0.001%-20%, 0.001%-15%, 0.001%-10%, 0.01%-20%, 0.01%-15%, 0.1%-25, 0.01%-10%, 1%-20%, 1%-15%, 1%-10%, or about 5%, about 10%, about 15%, about 20% of a particular nucleotide in a self-replicating RNA molecule are modified nucleotides. It is preferred that less than 100% of the nucleotides in a self-replicating RNA molecule are modified nucleotides. It is also preferred that less than 100% of a particular nucleotide in a self-replicating RNA molecule are modified nucleotides. Thus, preferred self-replicating RNA molecules comprise at least some unmodified nucleotides.

Self-replicating RNA molecules that comprise at least one modified nucleotide can be prepared using any suitable method. Several suitable methods are known in the art for producing RNA molecules that contain modified nucleotides. For example, a self-replicating RNA molecule that contains modified nucleotides can be prepared by transcribing (e.g., in vitro transcription) a DNA that encodes the self-replicating RNA molecule using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction will contain nucleotides and modified nucleotides, and other components that support the activity of the selected polymerase, such as a suitable buffer, and suitable salts. The incorporation of nucleotide analogs into a self-replicating RNA may be engineered, for example, to alter the stability of such RNA molecules, to increase resistance against RNases, to establish replication after introduction into appropriate host cells (“infectivity” of the RNA), and/or to induce or reduce innate and adaptive immune responses. Suitable synthetic methods can be used alone, or in combination with one or more other methods (e.g., recombinant DNA or RNA technology), to produce a self-replicating RNA molecule that contain one or more modified nucleotides.

Nucleic acid synthesis can also be performed using suitable recombinant methods that are well-known and conventional in the art, including cloning, processing, and/or expression of polynucleotides and gene products encoded by such polynucleotides. DNA shuffling by random fragmentation and PCR reassembly of gene fragments and synthetic polynucleotides are examples of known techniques that can be used to design and engineer polynucleotide sequences. Site-directed mutagenesis can be used to alter nucleic acids and the encoded proteins, for example, to insert new restriction sites, alter glycosylation patterns, change codon preference, produce splice variants, introduce mutations and the like.

In some embodiments, nucleic acids provided herein encode for an RNA polymerase. In some embodiments, nucleic acids provided herein encode for a viral RNA polymerase. In some embodiments, nucleic acids provided herein encode for: (1) a viral RNA polymerase; and (2) a cancer-associated protein or a functional fragment thereof. In some embodiments, compositions provided herein comprise a first nucleic acid encoding for a viral RNA polymerase; and a second nucleic acid encoding for a cancer-associated protein or a functional fragment thereof. In some embodiments, nucleic acids provided herein encode for: (1) a viral RNA polymerase; and (2) a cancer therapeutic antibody or a functional fragment thereof. In some embodiments, compositions provided herein comprise a first nucleic acid encoding for a viral RNA polymerase; and a second nucleic acid encoding for a cancer therapeutic antibody or a functional fragment thereof.

Provided herein are compositions comprising a self-replicating RNA. A self-replicating RNA (also called a replicon) includes any genetic element, for example, a plasmid, cosmid, bacmid, phage or virus that is capable of replication largely under its own control. Self-replication provides a system for self-amplification of nucleic acids provided herein in mammalian cells. In some embodiments, the self-replicating RNA is single stranded. In some embodiments, the self-replicating RNA is double stranded.

In some embodiments, a nucleic acid described herein comprises a sequence encoded for an infectious disease antigen described here and for an RNA-dependent RNA polymerase. In some embodiments, the RNA-dependent RNA polymerase is a VEEV RNA polymerase. In some embodiments, the two nucleic acid coding elements are present in separate nucleic acids. In some embodiments, the two nucleic acid coding elements are present on the same nucleic acid. An RNA polymerase provided herein can include but is not limited to: an alphavirus RNA polymerase, an Eastern equine encephalitis virus (EEEV) RNA polymerase, a Western equine encephalitis virus (WEEV), Venezuelan equine encephalitis virus (VEEV), Also, Chikungunya virus (CHIKV), Semliki Forest virus (SFV), or Sindbis virus (SINV). In some embodiments, the RNA polymerase is a VEEV RNA polymerase. In some embodiments, the nucleic acid encoding for the RNA polymerase comprises at least 85% identity to the nucleic acid sequence of SEQ ID NO: 71. In some embodiments, the nucleic acid encoding for the RNA polymerase comprises at least 90% identity to the nucleic acid sequence of SEQ ID NO: 71. In some embodiments, the nucleic acid encoding for the RNA polymerase comprises at least 95% identity to the nucleic acid sequence of SEQ ID NO: 71. In some embodiments, the nucleic acid encoding for the RNA polymerase comprises at least 99% identity to the nucleic acid sequence of SEQ ID NO: 71. In some embodiments, the nucleic acid encoding for the RNA polymerase is SEQ ID NO: 71.

In some embodiments, the amino acid sequence for VEEV RNA polymerase comprises at least 85% identity to RELPVLDSAAFNVECFKKYACNNEYWETFKENPIRLTEENVVNYITKLKGP (SEQ ID NO: 72), TQMRELPVLDSAAFNVECFKKYACNNEYWETFKENPIRLTE (SEQ ID NO: 73), or SEQ ID NO: 74. In some embodiments, the amino acid sequence for VEEV RNA polymerase comprises at least 90% identity to SEQ ID NO: 72, SEQ ID NO: 73, or SEQ ID NO: 74. In some embodiments, the amino acid sequence for VEEV RNA polymerase comprises at least 95% identity to SEQ ID NO: 72, SEQ ID NO: 73, or SEQ ID NO: 74. In some embodiments, the amino acid sequence for VEEV RNA polymerase comprises at least 99% identity SEQ ID NO: 72, SEQ ID NO: 73, or SEQ ID NO: 74. In some embodiments, the amino acid sequence for VEEV RNA polymerase is SEQ ID NO: 72, SEQ ID NO: 73, or SEQ ID NO: 74.

Provided herein are compositions and methods comprising replicon RNA (repRNA) encoding for one or more structural proteins from a non-enveloped virus. In some embodiments, the repRNA encodes a protease. In some embodiments, the repRNA encodes the 3CD protease. In some embodiments, the structural protein and the protease are co-expressed. In further embodiments, the repRNA comprises one or more open reading frames. In some embodiments, the open reading frames are separated by an internal ribosomal entry site (IRES). In some embodiments, the open reading frames are separated by a ribosomal skipping peptide sequence. In some embodiments the ribosomal skipping peptide sequence is from Thosea asigna virus (T2A).

### Nanoparticles

Provided herein are various compositions comprising a nanoparticle. In some embodiments, the nanoparticle comprises a lipid carrier. Nanoparticles are abbreviated as NPs herein. Nanoparticles provided herein may be an organic, inorganic, or a combination of inorganic and organic materials that are less than about 1 micrometer (pm) in diameter. In some embodiments, nanoparticles provided herein are used as a delivery system for a bioactive agent provided herein (e.g., a nucleic acid encoding a cancer-associated protein, or a cancer therapeutic antibody). Further provided herein are various compositions comprising lipid carrier complexes or nanoparticle-complexes, wherein a plurality of lipid carriers or a plurality of nanoparticles interact physically, chemically, and/or covalently. The specific type of interaction between lipid carriers or between nanoparticles will depend upon the characteristic shapes, sizes, chemical compositions, physical properties, and physiologic properties. Nanoparticles provided herein can include but are not limited to: oil in water emulsions, nanostructured lipid carriers (NLCs), cationic nanoemulsions (CNEs), vesicular phospholipid gels (VPG), polymeric nanoparticles, cationic lipid nanoparticles, liposomes, gold nanoparticles, solid lipid nanoparticles (LNPs or SLNs), mixed phase core NLCs, ionizable lipid carriers, magnetic carriers, polyethylene glycol (PEG)-functionalized carriers, cholesterol-functionalized carriers, polylactic acid (PLA)-functionalized carriers, and polylactic-co-glycolic acid (PLGA)-functionalized lipid carriers.

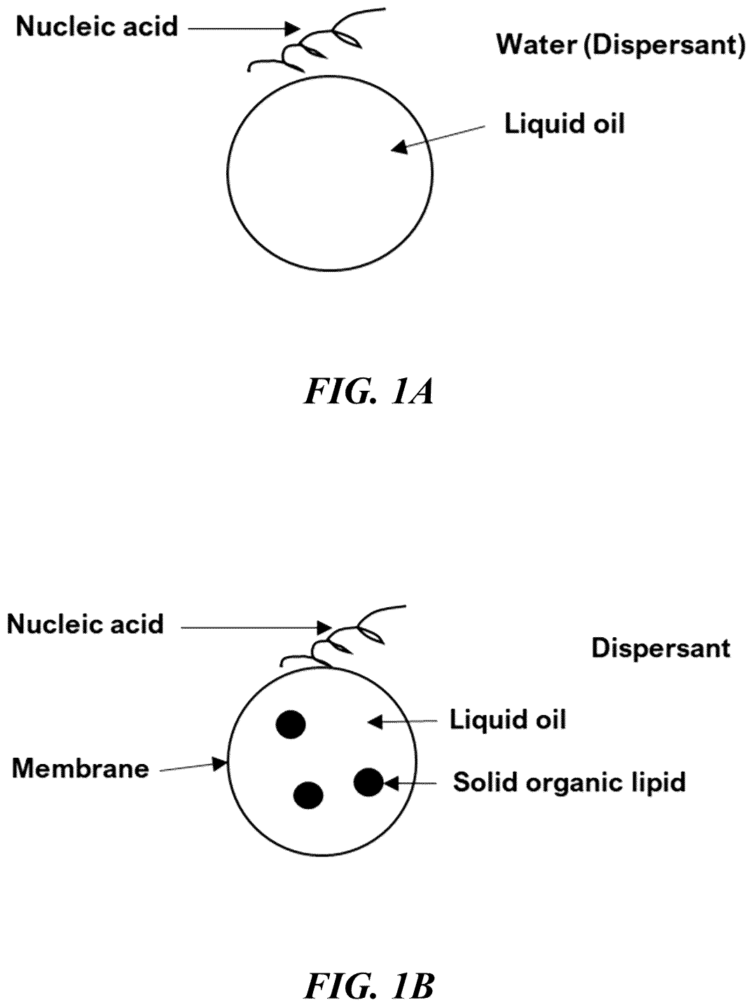

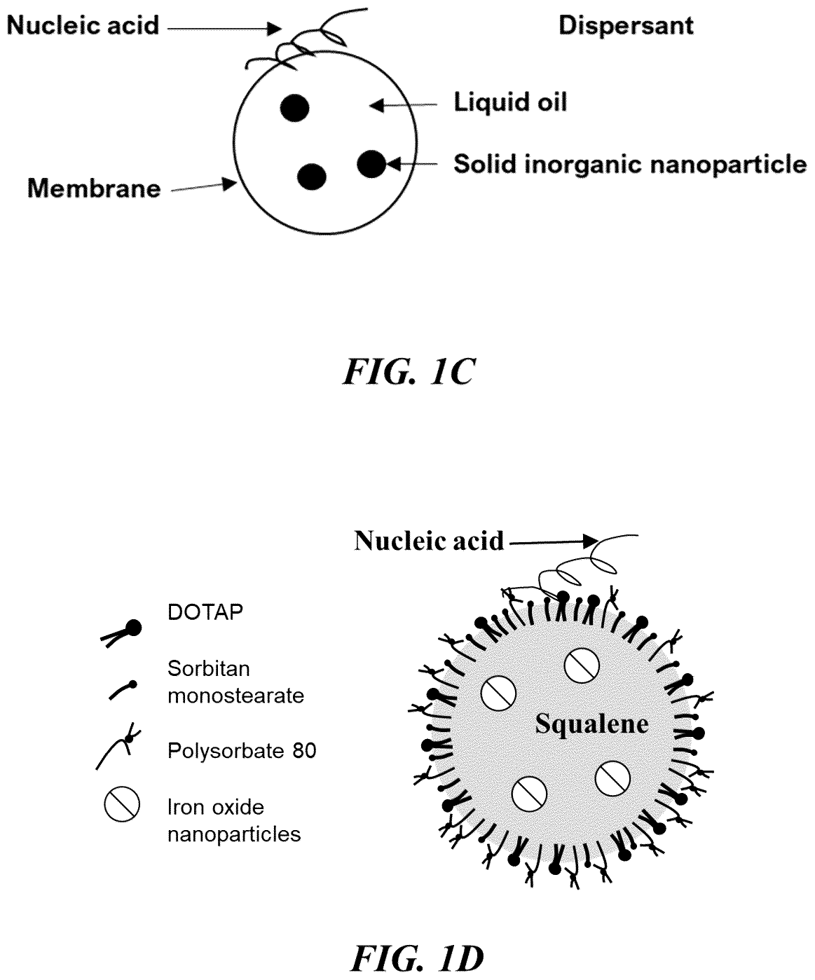

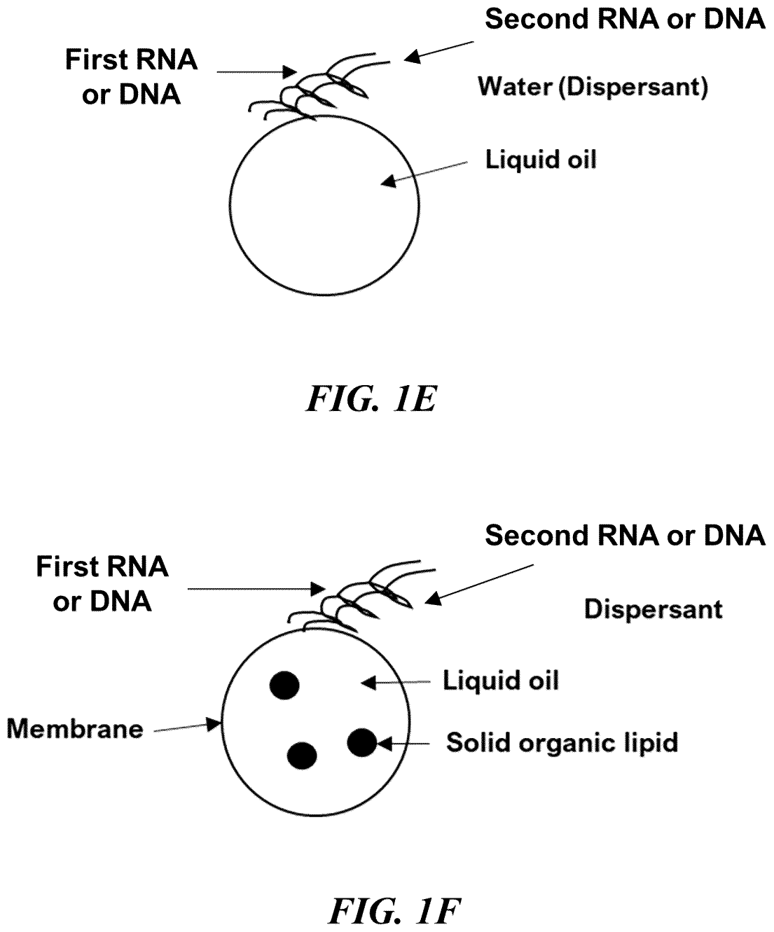

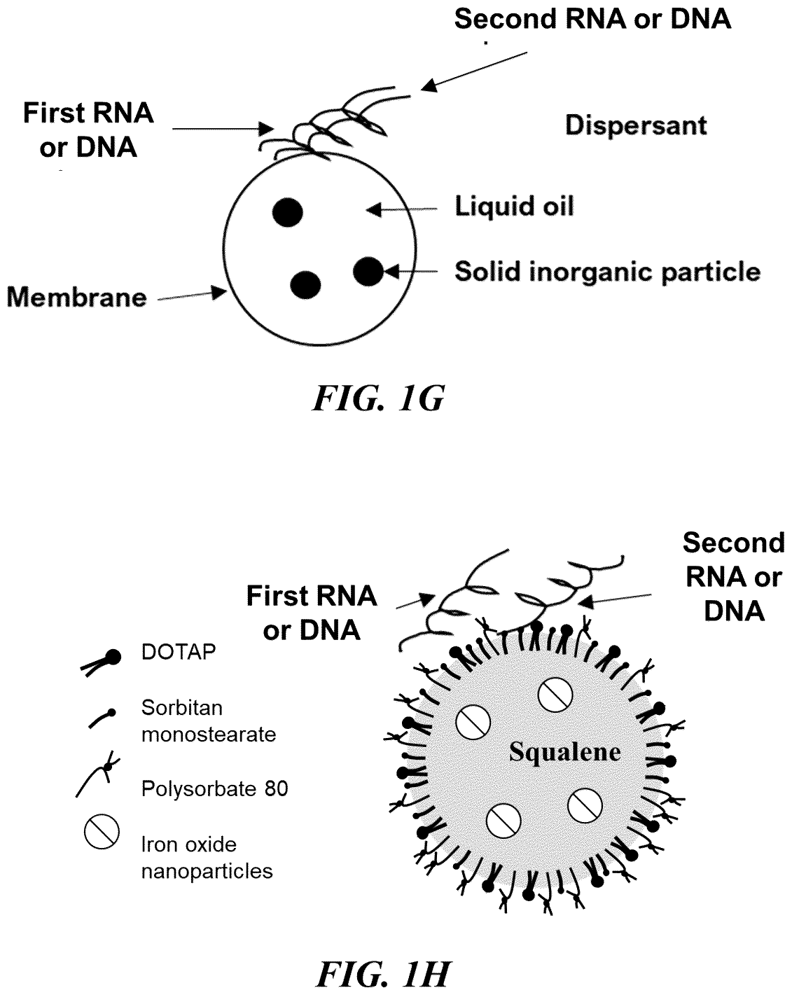

Various nanoparticles and formulations of nanoparticles (i.e., nanoemulsions) are employed. Exemplary nanoparticles are illustrated in FIGS. 1A-1H. Oil in water emulsions, as illustrated in FIG. 1A (not to scale), are stable, immiscible fluids containing an oil droplet dispersed in water or aqueous phase. FIG. 1B (not to scale) illustrates a nanostructured lipid carrier (NLCs) which can comprise a blend of solid organic lipids (e.g., trimyristin) and liquid oil (e.g., squalene). In NLCs, the solid lipid is dispersed in the liquid oil. The entire nanodroplet is dispersed in the aqueous (water) phase. In some embodiments, the nanoparticle comprises inorganic nanoparticles, as illustrated in FIG. 1C (not to scale), as solid inorganic nanoparticles (e.g., iron oxide nanoparticles) dispersed in liquid oil. FIG. 1D (not to scale) illustrates a nanoparticle comprising a cationic lipid membrane and a liquid oil without an inorganic particle. Nucleic acids provided herein can be complexed with a nanoparticle in Table 3 in cis (FIGS. 1A-1D) or in trans (FIGS. 1E-1H). For example, a first RNA or DNA molecule can comprise a plurality of cancer-associated proteins and a second RNA or DNA molecule can comprise an RNA polymerase complex. As another example, a first RNA or DNA molecule can comprise one or more cancer-associated proteins and a RNA polymerase on the same nucleic acid; and a second RNA or DNA molecule can comprise an additional cancer-associated protein and/or an RNA polymerase.

Provided herein are nanoemulsions and nanodroplets comprising a plurality of lipid carriers or nanoparticles, wherein each lipid carrier or nanoparticle comprises a cationic lipid. In some embodiments, nanoemulsions comprises a plurality of cationic lipid carriers. In some embodiments, a composition provided herein comprises a cationic nanoemulsion. In some embodiments, cationic nanoemulsions described herein comprise a lipid (or other surfactant) molecules surrounding an oil particle that is dispersed in water and give the oil particle a cationic (positively charged) surface to which negatively-charged RNA molecules can adhere.

The entire nanodroplet can be dispersed as a colloid in the aqueous (water) phase or in a suspension. In some embodiments, nanoparticles provided herein are dispersed in an aqueous solution. Non-limiting examples of aqueous solutions include water (e.g., sterilized, distilled, deionized, ultra-pure, RNAse-free, etc.), saline solutions (e.g., Kreb's, Ascaris, Dent's, Tet's saline), or 1% (w/v) dimethyl sulfoxide (DMSO) in water.

In some embodiments, nanoparticles provided herein comprise a hydrophilic surface. In some embodiments, the hydrophilic surface comprises a cationic lipid. In some embodiments, the hydrophilic surface comprises an ionizable lipid. In some embodiments, the nanoparticle comprises a membrane. In some embodiments, the membrane comprises a cationic lipid. In some embodiments, the nanoparticles provided herein comprise a cationic lipid. Exemplary cationic lipids for inclusion in the hydrophilic surface include, without limitation: 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxy-dodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis (azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″′,9″″′-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9″′Z,12Z,12′Z,12″Z,12″′Z)-tetrakis(octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Other examples for suitable classes of lipids include, but are not limited to, the phosphatidylcholines (PCs), phosphatidylethanolamines (PEs), phosphatidylglycerol (PGs); and PEGylated lipids including PEGylated version of any of the above lipids (e.g., DSPE-PEGs). In some embodiments, the nanoparticle provided herein comprises DOTAP.

In some embodiments, the nanoparticle provided herein comprises a hydrophobic lipid core. In some embodiments, the hydrophobic lipid core is in liquid phase at 25 degrees C. Non-limiting examples of hydrophobic lipid core components that can be used include α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E. In some embodiments, the nanoparticle provided herein comprises a triglyceride. Exemplary triglycerides include but are not limited to: capric triglycerides, caprylic triglycerides, a caprylic and capric triglycerides, triglyceride esters, and myristic acid triglycerins. In some embodiments, the hydrophobic lipid is in solid phase. In some embodiments, the hydrophobic lipid is in liquid phase, also referred to as an oil. In some embodiments, the hydrophobic lipid comprises squalene. In some embodiments, the hydrophobic lipid comprises solanesol.

In some embodiments, the nanoparticles provided herein comprise a liquid organic material and a solid inorganic material. In some embodiments, the nanoparticle provided herein comprises an inorganic particle. In some embodiments, the inorganic particle is a solid inorganic particle. In some embodiments, the nanoparticle provided herein comprises the inorganic particle within the hydrophobic core. In some embodiments, the nanoparticle provided herein comprises a metal. In some embodiments, the nanoparticle provided herein comprises a metal within the hydrophobic core. The metal can be without limitation, a metal salt such as a transition metal salt, a metal oxide such as a transition metal oxide, a metal hydroxide such as a transition metal hydroxide, a metal phosphate such as a transition metal phosphate, or a metalloid (e.g., silicon and silicon-based compounds or alloys). In some embodiments, the nanoparticle provided herein comprises aluminum oxide (Al₂O₃), aluminum oxyhydroxide, iron oxide (Fe₃O₄, Fe₂O₃, FeO, or combinations thereof), titanium dioxide, silicon dioxide (SiO₂), aluminum hydroxyphosphate (Al(OH)ₓ(PO₄)_{y}), calcium phosphate (Ca₃(PO₄)₂), calcium hydroxyapatite (Ca₁₀(PO₄)₆(OH)₂), iron gluconate, or iron sulfate. The inorganic particles may be formed from one or more same or different metals (any metals including transition metal). In some embodiments, the inorganic particle is a transition metal oxide. In some embodiments, the transition metal is magnetite (Fe₃O₄), maghemite (y—Fe₂O₃), wūstite (FeO), or hematite (alpha (α)—Fe₂O₃). In some embodiments, the metal is aluminum hydroxide or aluminum oxyhydroxide, and a phosphate-terminated lipid or a surfactant, such as oleic acid, oleylamine, SDS, TOPO or DSPA is used to coat the inorganic solid nanoparticle, before it is mixed with the liquid oil to form the hydrophobic core. In some embodiments, the metal can comprise a paramagnetic, a superparamagnetic, a ferrimagnetic or a ferromagnetic compound. In some embodiments, the metal is a superparamagnetic iron oxide (Fe₃O₄).

In some embodiments, nanoparticles provided herein comprise a cationic lipid, an oil, and an inorganic particle. In some embodiments, the nanoparticle provided herein comprises DOTAP; squalene and/or glyceryl trimyristate-dynasan; and iron oxide. In some embodiments, the nanoparticle provided herein further comprises a surfactant.

In some embodiments, nanoparticles provided herein comprise a cationic lipid, an oil, an inorganic particle, and a surfactant.

Surfactants are compounds that lower the surface tension between two liquids or between a liquid and a solid component of the nanoparticles provided herein. Surfactants can be hydrophobic, hydrophilic, or amphiphilic. In some embodiments, the nanoparticle provided herein comprises a hydrophobic surfactant. Exemplary hydrophobic surfactants that can be employed include but are not limited to: sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), and sorbitan trioleate (SPAN® 85).

Suitable hydrophobic surfactants include those having a hydrophilic-lipophilic balance (HLB) value of 10 or less, for instance, 5 or less, from 1 to 5, or from 4 to 5. For instance, the hydrophobic surfactant can be a sorbitan ester having an HLB value from 1 to 5, or from 4 to 5. In some embodiments, nanoparticles provided herein comprise a ratio of the esters that yields a hydrophilic-lipophilic balance between 8 and 11. HLB is used to categorize surfactants as hydrophilic or lipophilic. The HLB scale provides for the classification of surfactant function calculated e.g., by Griffin's method:

$$HLB = \frac{20 {M}_{h}}{M} ,$$

where Mₕ is the molecular mass of the hydrophilic portion of the lipid carrier and M is the molecular mass of the lipid carrier. The HLB scale is provided below:
HLB=0: fully lipophilic/hydrophobic carrier;
HLB between 0 and 6 is an oil soluble carrier;
HLB between 6 and 9 is a water dispersible carrier;
HLB between 9 and 20 is a hydrophilic, water soluble carrier;
HLB=20: fully hydrophilic/lipophobic carrier.

In some embodiments, a nanoparticle or a lipid carrier provided herein comprises a hydrophilic surfactant, also called an emulsifier. In some embodiments, a nanoparticle or a lipid carrier provided herein comprises polysorbate. Polysorbates are oily liquids derived from ethoxylated sorbitan (a derivative of sorbitol) esterified with fatty acids. Exemplary hydrophilic surfactants that can be employed include but are not limited to: polysorbates such as TWEEN®, Kolliphor, Scattics, Alkest, or Canarcel; polyoxyethylene sorbitan ester (polysorbate); polysorbate 80 (polyoxyethylene sorbitan monooleate, or TWEEN® 80); polysorbate 60 (polyoxyethylene sorbitan monostearate, or TWEEN® 60); polysorbate 40 (polyoxyethylene sorbitan monopalmitate, or TWEEN® 40); and polysorbate 20 (polyoxyethylene sorbitan monolaurate, or TWEEN® 20). In one embodiment, the hydrophilic surfactant is polysorbate 80.

In some embodiments, nanoparticles and lipid carriers provided herein comprise a hydrophobic core surrounded by a lipid membrane (e.g., a cationic lipid such as DOTAP). In some embodiments, the hydrophobic core comprises: one or more inorganic particles; a phosphate-terminated lipid; and a surfactant.

Inorganic solid nanoparticles described herein can be surface modified before mixing with the liquid oil. For instance, if the surface of the inorganic solid nanoparticle is hydrophilic, the inorganic solid nanoparticle may be coated with hydrophobic molecules (or surfactants) to facilitate the miscibility of the inorganic solid nanoparticle with the liquid oil in the “oil” phase of the nanoemulsion particle. In some embodiments, the inorganic particle is coated with a capping ligand, the phosphate-terminated lipid, and/or the surfactant. In some embodiments the hydrophobic core comprises a phosphate-terminated lipid. Exemplary phosphate-terminated lipids that can be employed include but are not limited to: trioctylphosphine oxide (TOPO) or distearyl phosphatidic acid (DSPA). In some embodiments, the hydrophobic core comprises a surfactant such as a phosphorous-terminated surfactant, a carboxylate-terminated surfactant, a sulfate-terminated surfactant, or an amine-terminated surfactant. Exemplary carboxylate-terminated surfactants include oleic acid. Typical amine terminated surfactants include oleylamine. In some embodiments, the surfactant is distearyl phosphatidic acid (DSPA), oleic acid, oleylamine or sodium dodecyl sulfate (SDS). In some embodiments, the inorganic solid nanoparticle is a metal oxide such as an iron oxide, and a surfactant, such as oleic acid, oleylamine, SDS, DSPA, or TOPO, is used to coat the inorganic solid nanoparticle, before it is mixed with the liquid oil to form the hydrophobic core.

In some embodiments, the hydrophobic core comprises: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core comprises: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the hydrophobic core consists of: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core consists of: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80. In some embodiments, the nanoparticle provided herein can comprise from about 0.2% to about 40% w/v squalene, from about 0.001% to about 10% w/v iron oxide nanoparticles, from about 0.2% to about 10% w/v DOTAP, from about 0.25% to about 5% w/v sorbitan monostearate, and from about 0.5% to about 10% w/v polysorbate 80. In some embodiments the nanoparticle provided herein from about 2% to about 6% w/v squalene, from about 0.01% to about 1% w/v iron oxide nanoparticles, from about 0.2% to about 1% w/v DOTAP, from about 0.25% to about 1% w/v sorbitan monostearate, and from about 0.5%) to about 5% w/v polysorbate 80. In some embodiments, the nanoparticle provided herein can comprise from about 0.2% to about 40% w/v squalene, from about 0.001% to about 10% w/v aluminum hydroxide or aluminum oxyhydroxide nanoparticles, from about 0.2% to about 10% w/v DOTAP, from about 0.25% to about 5% w/v sorbitan monostearate, and from about 0.5% to about 10% w/v polysorbate 80. In some embodiments, the nanoparticle provided herein can comprise from about 2% to about 6% w/v squalene, from about 0.01% to about 1% w/v aluminum hydroxide or aluminum oxyhydroxide nanoparticles, from about 0.2% to about 1% w/v DOTAP, from about 0.25% to about 1% w/v sorbitan monostearate, and from about 0.5%) to about 5% w/v polysorbate 80.

In some embodiments, a composition described herein comprises at least one nanoparticle formulation as described in Table 3. In some embodiments, a composition described herein comprises any one of NP-1 to NP-30. In some embodiments, a composition described herein comprises any one of NP-1 to NP-34. In some embodiments, the nanoparticles provided herein are admixed with a nucleic acid provided herein. In some embodiments, nanoparticles provided herein are made by homogenization and ultrasonication techniques.

TABLE 3
Nanoparticle Formulations.

| | Cationic Lipid(s) | Oil(s) | Surfactant(s) | Additional Ingredients |
|---|---|---|---|---|
| Name | % (w/v) or mg/ml | % (w/v) or mg/ml | % (w/v) or mg/ml | % (w/v), mg/ml, or mM |
| NP-1 | 30 mg/ml 1,2-dioleoyl-3- | 37.5 mg/ml squalene | 37 mg/ml sorbitan | 0.2 mg Fe/ml 12 nm oleic |
| | trimethylammonium-propane | | monostearate, (2R)- | acid-coated iron oxide |
| | (DOTAP) chloride | | 2-[(2R,3R,4S)-3,4- | nanoparticles |
| | | | Dihydroxyoxolan-2- | 10 mM sodium citrate |
| | | | yl]-2-hydroxyethyl | dihydrate. |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-2 | 30 mg/ml 1,2-dioleoyl-3- | 37.5 mg/ml squalene | 37 mg/ml sorbitan | 1 mg Fe/ml 15 nm oleic |
| | trimethylammonium-propane | | monostearate (2R)- | acid-coated iron oxide |
| | (DOTAP) chloride | | 2-[(2R,3R,4S)-3,4- | nanoparticles |
| | | | Dihydroxyoxolan-2- | 10 mM sodium citrate |
| | | | yl]-2-hydroxyethyl | dihydrate |
| | | | octadecenoate | |
| | | | C₂₄H₄₆O₆ | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-3 | 30 mg/ml 1,2-dioleoyl-3- | 37.5 mg/ml Miglyol | 37 mg/ml sorbitan | 0.2 mg Fe/ml 15 nm oleic |
| | trimethylammonium-propane | 812N | monostearate, (2R)- | acid-coated iron oxide |
| | (DOTAP) chloride | (triglyceride ester of | 2-[(2R,3R,4S)-3,4- | nanoparticles |
| | | saturated | Dihydroxyoxolan-2- | 10 mM sodium citrate |
| | | coconut/palmkernel | yl]-2-hydroxyethyl | dihydrate |
| | | oil derived caprylic | octadecenoate | |
| | | and capric fatty acids | C₂₄H₄₆O₆ | |
| | | and plant derived | (SPAN ® 60) | |
| | | glycerol) | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-4 | 30 mg/ml 1,2-dioleoyl-3- | 37.5 mg/ml Miglyol | 37 mg/ml sorbitan | 1 mg Fe/ml 15 nm oleic |
| | trimethylammonium-propane | 812N | monostearate, (2R)- | acid-coated iron oxide |
| | (DOTAP) chloride | (triglyceride ester of | 2-[(2R,3R,4S)-3,4- | nanoparticles |
| | | saturated | Dihydroxyoxolan-2- | 10 mM sodium citrate |
| | | coconut/palmkernel | yl]-2-hydroxyethyl | dihydrate. |
| | | oil derived caprylic | octadecenoate, | |
| | | and capric fatty acids | C₂₄H₄₆O₆) | |
| | | and plant derived | (SPAN ® 60) | |
| | | glycerol) | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-5 | 30 mg/ml DOTAP chloride | 37.5 mg/ml squalene | 37 mg/ml sorbitan | 1 mg/ml trioctylphosphine |
| | | | monostearate | oxide (TOPO)-coated |
| | | | (SPAN ® 60) | aluminum hydroxide |
| | | | 37 mg/ml | (Alhydrogel ® 2%) |
| | | | polysorbate 80 | particles |
| | | | (TWEEN ® 80) | 10 mM sodium citrate |
| | | | | dihydrate |
| NP-6 | 30 mg/ml DOTAP chloride | 37.5 mg/ml Solanesol | 37 mg/ml sorbitan | 0.2 mg Fe/ml oleic acid- |
| | | (Cayman chemicals) | monostearate | coated iron oxide |
| | | | (SPAN ® 60) | nanoparticles |
| | | | 37 mg/ml | 10 mM sodium citrate |
| | | | polysorbate 80 | |
| | | | (TWEEN ® 80) | |
| NP-7 | 30 mg/ml DOTAP chloride | 37.5 mg/ml squalene | 37 mg/ml sorbitan | 10 mM sodium citrate |
| | | 2.4 mg/ml Dynasan | monostearate | |
| | | 114 | (SPAN ® 60) | |
| | | | 37 mg/ml | |
| | | | polysorbate 80 | |
| | | | (TWEEN ® 80) | |
| NP-8 | 4 mg/ml DOTAP chloride | 43 mg/ml squalene | 5 mg/ml sorbitan | 10 mM sodium citrate |
| | | | trioleate (SPAN ® | |
| | | | 85) | |
| | | | 5 mg/ml polysorbate | |
| | | | 80 (TWEEN ® 80) | |
| NP-9 | 7.5 mg/ml 1,2-dioleoyl-3- | 9.4 mg/ml squalene | 9.3 mg/ml sorbitan | 0.05 mg/ml 15 nanometer |
| | trimethylammonium-propane | ((6E,10E,14E,18E)- | monostearate (2R)- | superparamagnetic iron |
| | (DOTAP) chloride | 2,6,10,15,19,23- | 2-[(2R,3R,4S)-3,4- | oxide (Fe₃O₄) |
| | | Hexamethyltetracosa- | Dihydroxyoxolan-2- | 10 mM sodium citrate |
| | | 2,6,10,14,18,22- | yl]-2-hydroxyethyl | dihydrate |
| | | hexaene, C₃₀H₅₀) | octadecenoate, | |
| | | 0.63 mg/ml glyceryl | C₂₄H₄₆O₆) | |
| | | trimyristate-dynasan | (SPAN ® 60) | |
| | | (DYNASAN 114 ®) | 9.3 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-10 | 0.4% DOTAP | 0.25% glyceryl | 0.5% sorbitan | |
| | | trimyristate-dynasan | monostearate | |
| | | (DYNASAN 114 ®) | (SPAN ® 60) | |
| | | 4.75% Squalene | 0.5% polysorbate 80 | |
| | | | (TWEEN ® 80) | |
| NP-11 | 3.0% DOTAP | 0.25% glyceryl | 3.7% sorbitan | |
| | | trimyristate-dynasan | monostearate | |
| | | (DYNASAN 114 ®) | (SPAN ® 60) | |
| | | 3.75% Squalene | 3.7% polysorbate 80 | |
| | | | (TWEEN ® 80) | |
| NP-12 | 0.4% DOTAP | 4.3% Squalene | 0.5% sorbitan | |
| | | | trioleate (SPAN ® | |
| | | | 85) | |
| | | | 0.5% polysorbate 80 | |
| | | | (TWEEN ® 80) | |
| NP-13 | 0.4% DOTAP | 0.25% glyceryl | 2.0% polysorbate 80 | |
| | | trimyristate-dynasan | (TWEEN ® 80) | |
| | | (DYNASAN 114 ®) | | |
| | | 4.08% squalene | | |
| NP-14 | 0.4% DOTAP | 0.25% glyceryl | 0.5% sorbitan | |
| | | trimyristate-dynasan | trioleate (SPAN ® | |
| | | (DYNASAN 114 ®) | 85) | |
| | | 4.08% squalene | 2.0% polysorbate 80 | |
| | | | (TWEEN ® 80) | |
| NP-15 | 0.4% DOTAP | 0.25% glyceryl | 0.25% sorbitan | |
| | | trimyristate-dynasan | trioleate (SPAN ® | |
| | | (DYNASAN 114 ®) | 85) | |
| | | 4.08% squalene | 2.0% polysorbate 80 | |
| | | | (TWEEN ® 80) | |
| NP-16 | 0.4% DOTAP | 5% squalene | 0.5% sorbitan | |
| | | | trioleate (SPAN ® | |
| | | | 85) | |
| | | | 2.0% polysorbate 80 | |
| | | | (TWEEN ® 80) | |
| NP-17 | 0.4% DOTAP | 5% squalene | 0.5% sorbitan | |
| | | | monostearate | |
| | | | (SPAN ® 60) | |
| | | | 2% polysorbate 80 | |
| | | | (TWEEN ® 80) | |
| NP-18 | 0.4% DOTAP | 0.25% glyceryl | 2% sorbitan trioleate | |
| | | trimyristate-dynasan | (SPAN ® 85) | |
| | | (DYNASAN 114 ®) | 2% polysorbate 80 | |
| | | 4.08% squalene | (TWEEN ® 80) | |
| NP-19 | 0.4% DOTAP | 0.25% glyceryl | 0.5% sorbitan | 1% aluminum hydroxide |
| | | trimyristate-dynasan | monostearate | |
| | | (DYNASAN 114 ®) | (SPAN ® 60) | |
| | | 4.75% Squalene | 0.5% polysorbate 80 | |
| | | | (TWEEN ® 80) | |
| NP-20 | 3.0% DOTAP | 0.25% glyceryl | 3.7% sorbitan | 1% aluminum hydroxide |
| | | trimyristate-dynasan | monostearate | |
| | | (DYNASAN 114 ®) | (SPAN ® 60) | |
| | | 3.75% Squalene | 3.7% polysorbate 80 | |
| | | | (TWEEN ® 80) | |
| NP-21 | 0.4% DOTAP | 4.3% Squalene | 0.5% sorbitan | 1% aluminum hydroxide |
| | | | trioleate (SPAN ® | |
| | | | 85) | |
| | | | 0.5% polysorbate 80 | |
| | | | (TWEEN ® 80 | |
| NP-22 | 0.4% DOTAP | 0.25% glyceryl | 2.0% polysorbate 80 | 1% aluminum hydroxide |
| | | trimyristate-dynasan | (TWEEN ® 80) | |
| | | (DYNASAN 114 ®) | | |
| | | 4.08% squalene | | |
| NP-23 | 0.4% DOTAP | 0.25% glyceryl | 0.5% sorbitan | 1% aluminum hydroxide |
| | | trimyristate-dynasan | trioleate (SPAN ® | |
| | | (DYNASAN 114 ®) | 85) | |
| | | 4.08% squalene | 2.0% polysorbate 80 | |
| | | | (TWEEN ® 80 | |
| NP-24 | 0.4% DOTAP | 0.25% glyceryl | 0.25% sorbitan | 1% aluminum hydroxide |
| | | trimyristate-dynasan | trioleate (SPAN ® | |
| | | (DYNASAN 114 ®) | 85) | |
| | | 4.08% squalene | 2.0% polysorbate 80 | |
| | | | (TWEEN ® 80) | |
| NP-25 | 0.4% DOTAP | 5% squalene | 0.5% sorbitan | 1% aluminum hydroxide |
| | | | trioleate (SPAN ® | |
| | | | 85) | |
| | | | 2.0% polysorbate 80 | |
| | | | (TWEEN ® 80) | |
| NP-26 | 0.4% DOTAP | 5% squalene | 0.5% sorbitan | 1% aluminum hydroxide |
| | | | monostearate | |
| | | | (SPAN ® 60) | |
| | | | 2% polysorbate 80 | |
| | | | (TWEEN ® 80) | |
| NP-27 | 0.4% DOTAP | 0.25% glyceryl | 2% sorbitan trioleate | 1% aluminum hydroxide |
| | | trimyristate-dynasan | (SPAN ® 85) | |
| | | (DYNASAN 114 ®) | 2% polysorbate 80 | |
| | | 4.08% squalene | (TWEEN ® 80) | |
| NP-28 | 0.5-5.0 mg/ml DOTAP | 0.2-10% (v/v) | 0.01-2.5% (v/v) | |
| | | squalene | polysorbate 80 | |
| | | | (TWEEN ® 80) | |
| NP-29 | 0.4% (w/w) DOTAP | 4.3% (w/w) squalene | 0.5% (w/w) sorbitan | |
| | | | trioleate (SPAN ® | |
| | | | 85) | |
| | | | 0.5% (w/w) | |
| | | | polysorbate 80 | |
| | | | (TWEEN ® 80) | |
| NP-30 | 30 mg/ml DOTAP chloride | 37.5 mg/ml squalene | 37 mg/ml sorbitan | 10 mM sodium citrate |
| | | | monostearate | |
| | | | (SPAN ® 60) | |
| | | | 37 mg/ml | |
| | | | polysorbate 80 | |
| | | | (TWEEN ® 80) | |
| NP-31 | 30 mg/ml DOTAP chloride | 37.5 mg/ml squalene | 37 mg/ml sorbitan | 0.4 mg Fe/ml 5 nm oleic |
| | | | monostearate | acid-coated iron oxide |
| | | | (SPAN ® 60) | nanoparticles |
| | | | 37 mg/ml | 10 mM sodium citrate |
| | | | polysorbate 80 | dihydrate |
| | | | (TWEEN ® 80) | |
| NP-32 | 0.8-1.6 mg/ml DOTAP | 4.5% squalene | 0.5% (w/w) sorbitan | 10 mM sodium citrate |
| | chloride | | trioleate (SPAN | |
| | | | 85 ®) | |
| | | | 0.5% (w/w) | |
| | | | polysorbate 80 | |
| | | | (TWEEN ® 80) | |
| NP-33 | 45-55 mol % ionizable cationic | 35-42 mol % | 1.25-1.75 mol % | |
| | lipid | cholesterol | PEG2000-DMG | |
| | 8-12 mol % | | | |
| | distearoylphosphatidylcholine | | | |
| | (DSPC) | | | |
| NP-34 | 50 mol % D-Lin-MC3-DMA | 38.5% cholesterol | 1.5% PEG-lipid | |
| | (MC3) | | | |
| | 10 mol % | | | |
| | distearoylphosphatidylcholine | | | |
| | (DSPC) | | | |

In some embodiments, nanoparticles provided herein comprise: sorbitan monostearate (e.g., SPAN®?60), polysorbate 80 (e.g., TWEEN®?80), DOTAP, squalene, and no solid particles. In some embodiments, nanoparticles provided herein comprise: sorbitan monostearate (e.g., SPAN®?60), polysorbate 80 (e.g., TWEEN®?80), DOTAP, squalene, and iron oxide particles. In some embodiments, nanoparticles provided herein comprise an immune stimulant. In some embodiments, the immune stimulant is squalene. In some embodiments, the immune stimulant is Miglyol 810 or Miglyol 812. Miglyol 810 is a triglyceride ester of saturated caprylic and capric fatty acids and glycerol. Miglyol 812 is a triglyceride ester of saturated coconut/palmkemnel oil derived caprylic and capric fatty acids and plant derived glycerol. In some embodiments, the immune stimulant can decrease the total amount of protein produced, but can increase the immune response to a composition provided herein (e.g., when delivered as a vaccine). In some embodiments, the immune stimulant can increase the total amount of protein produced, but can decrease the immune response to a composition provided herein.

Nanoparticles provided herein can be of various average diameters in size. In some embodiments, nanoparticles provided herein have an average diameter (z-average hydrodynamic diameter, measured by dynamic light scattering) ranging from about 20 nanometers (nm) to about 200 nm. In some embodiments, the z-average diameter of the nanoparticle ranges from about 20 nm to about 150 nm, from about 20 nm to about 100 nm, from about 20 nm to about 80 nm, from about 20 nm to about 60 nm. In some embodiments, the z-average diameter of the nanoparticle) ranges from about 40 nm to about 200 nm, from about 40 nm to about 150 nm, from about 40 nm to about 100 nm, from about 40 nm to about 90 nm, from about 40 nm to about 80 nm, or from about 40 nm to about 60 nm. In one embodiment, the z-average diameter of the nanoparticle is from about 40 nm to about 80 nm. In some embodiments, the z-average diameter of the nanoparticle is from about 40 nm to about 60 nm. In some embodiments, the nanoparticle is up to 100 nm in diameter. In some embodiments, the nanoparticle is 50 to 70 nm in diameter. In some embodiments, the nanoparticle is 40 to 80 nm in diameter. In some embodiments, the inorganic particle (e.g., iron oxide) within the hydrophobic core of the nanoparticle can be an average diameter (number weighted average diameter) ranging from about 3 nm to about 50 nm. For instance, the inorganic particle can have an average diameter of about 5 nm, about 10 nm, about 15 nm, about 20 nm, about 25 nm, about 30 nm, about 35 nm, about 40 nm, about 45 nm, or about 50 nm. In some embodiments, the ratio of esters and lipids yield a particle size between 30 nm and 200 nm. In some embodiments, the ratio of esters and lipids yield a particle size between 40 nm and 70 nm.

Nanoparticles provided herein may be characterized by the polydispersity index (PDI), which is an indication of their quality with respect to size distribution. In some embodiments, average polydispersity index (PDI) of the nanoparticles provided herein ranges from about 0.1 to about 0.5. In some embodiments, the average PDI of the nanoparticles can range from about 0.2 to about 0.5, from about 0.1 to about 0.4, from about 0.2 to about 0.4, from about 0.2 to about 0.3, or from about 0.1 to about 0.3.

In some embodiments, nanoparticles provided herein comprise an oil-to-surfactant molar ratio ranging from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, nanoparticles provided herein comprise a hydrophilic surfactant-to-lipid ratio ranging from about 0.1:1 to about 2:1, from about 0.2:1 to about 1.5:1, from about 0.3:1 to about 1:1, from about 0.5:1 to about 1:1, or from about 0.6:1 to about 1:1. In some embodiments, the nanoparticles provided herein comprise a hydrophobic surfactant-to-lipid ratio ranging from about 0.1:1 to about 5:1, from about 0.2:1 to about 3:1, from about 0.3:1 to about 2:1, from about 0.5:1 to about 2:1, or from about 1:1 to about 2:1.

In some embodiments, nanoparticles provided herein comprise from about 0.2% to about 40% w/v liquid oil, from about 0.0010% to about 10% w/v inorganic solid nanoparticle, from about 0.2% to about 10% w/v lipid, from about 0.25% to about 5% w/v hydrophobic surfactant, and from about 0.5% to about 10% w/v hydrophilic surfactant. In some embodiments, the lipid comprises a cationic lipid, and the oil comprises squalene, and/or the hydrophobic surfactant comprises sorbitan ester.

### Combination Compositions

Provided herein are compositions comprising a nanoparticle described herein and a nucleic acid encoding for a cancer-associated protein, or cancer-associated protein binding protein. In some embodiments, nucleic acids provided herein are incorporated, associated with, or complexed a lipid carrier provided herein to form a lipid carrier-nucleic acid complex. The lipid carrier-nucleic acid complex is formed via non-covalent interactions or via reversible covalent interactions.

Further provided herein is a nanoemulsion comprising a plurality of nanoparticles provided herein. In some embodiments, the nucleic acid further encodes for an RNA-dependent polymerase. In some embodiments, the RNA-dependent polymerase is a viral RNA polymerase. In some embodiments, the nucleic acid encoding for the RNA polymerase is on the same nucleic acid strand as the nucleic acid sequence encoding for the protein (e.g., cis). In some embodiments, the nucleic acid encoding for the RNA polymerase is on a different nucleic acid strand as the nucleic acid sequence encoding for the protein (e.g., trans). In some embodiments, the nucleic acid encoding for the RNA polymerase is a DNA molecule. In some embodiments, nucleic acid sequences encoding for a cancer-associated protein, a tumor antigen, a neoantigen, a cancer therapeutic antibody, or a functional fragment thereof are DNA or RNA molecules. In some embodiments, cancer-associated proteins and cancer therapeutic antibodies provided herein are encoded by DNA. Nanoparticles for inclusion include, without limitation, any one of NP-1 to NP-31, or any one of NP-1 to NP-34. Nucleic acids for inclusion include, without limitation, comprise a region comprising any one of, or a plurality of, SEQ ID NOS: 1, 2, 75, 76, 88, 89 and/or encodes for an amino acid sequence set forth in any one of SEQ ID NOS: 3 to 70, 77, 78, 87. In some instances, the nucleic acids further comprise a region encoding for an RNA polymerase, e.g., a region comprising a sequence of SEQ ID NO: 71.

Compositions provided herein can be characterized by an nitrogen:phosphate (N:P) molar ratio. The N:P ratio is determined by the amount of cationic lipid in the nanoparticle which contain nitrogen and the amount of nucleic acid used in the composition which contain negatively charged phosphates. A molar ratio of the lipid carrier to the nucleic acid can be chosen to increase the delivery efficiency of the nucleic acid, increase the ability of the nucleic acid-carrying nanoemulsion composition to elicit an immune response to the antigen, increase the ability of the nucleic acid-carrying nanoemulsion composition to elicit the production of antibody titers to the antigen in a subject. In some embodiments, compositions provided herein have a molar ratio of the lipid carrier to the nucleic acid can be characterized by the nitrogen-to-phosphate molar ratio, which can range from about 0.01:1 to about 1000:1, for instance, from about 0.2:1 to about 500:1, from about 0.5:1 to about 150:1, from about 1:1 to about 150:1, from about 1:1 to about 125:1, from about 1:1 to about 100:1, from about 1:1 to about 50:1, from about 1:1 to about 50:1, from about 5:1 to about 50:1, from about 5:1 to about 25:1, or from about 10:1 to about 20:1. In certain embodiments, the molar ratio of the lipid carrier to the nucleic acid, characterized by the nitrogen-to-phosphate (N:P) molar ratio, ranges from about 1:1 to about 150:1, from about 5:1 to about 25:1, or from about 10:1 to about 20:1. In one embodiment, the N:P molar ratio of the nanoemulsion composition is about 15:1. In some embodiments, the nanoparticle comprises a nucleic acid provided herein covalently attached to the membrane.

Compositions provided herein can be characterized by an oil-to-surfactant molar ratio. In some embodiments, the oil-to-surfactant ratio is the molar ratio of squalene: DOTAP, hydrophobic surfactant, and hydrophilic surfactant. In some embodiments, the oil-to-surfactant ratio is the molar ratio of squalene: DOTAP, sorbitan monostearate, and polysorbate 80. In some embodiments, the oil-to surfactant molar ratio ranges from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the oil-to-surfactant molar ratio is at least about 0.1:1, at least about 0.2:1, at least about 0.3:1, at least about 0.4:1, at least about 0.5:1, at least about 0.6:1, at least about 0.7:1. In some embodiments, the oil-to surfactant molar ratio is at least about 0.4:1 up to 1:1.

Compositions provided herein can be characterized by hydrophilic surfactant-to-lipid (e.g., cationic lipid) ratio. In some embodiments, the hydrophilic surfactant-to-lipid ratio ranges from about 0.1:1 to about 2:1, from about 0.2:1 to about 1.5:1, from about 0.3:1 to about 1:1, from about 0.5:1 to about 1:1, or from about 0.6:1 to about 1:1. Compositions provided herein can be characterized by hydrophobic surfactant-to-lipid (e.g., cationic lipid) ratio ranging. In some embodiments, the hydrophobic surfactant-to-lipid ratio ranges from about 0.1:1 to about 5:1, from about 0.2:1 to about 3:1, from about 0.3:1 to about 2:1, from about 0.5:1 to about 2:1, or from about 1:1 to about 2:1.

Provided herein is a dried composition comprising a sorbitan fatty acid ester, an ethoxylated sorbitan ester, a cationic lipid, an immune stimulant, and an RNA. Further provided herein are dried compositions, wherein the dried composition comprises sorbitan monostearate (e.g., SPAN® 60), polysorbate 80 (e.g., TWEEN® 80), DOTAP, an immune stimulant, and an RNA.

### Thermally Stable, Dried, and Lyophilized Cancer Vaccines

Provided herein are dried or lyophilized compositions and vaccines. Further provided herein are pharmaceutical compositions comprising a dried or lyophilized composition provided herein that is reconstituted in a suitable diluent and a pharmaceutically acceptable carrier. In some embodiments, the diluent is aqueous. In some embodiments, the diluent is water.

A lyophilized composition is generated by a low temperature dehydration process involving the freezing of the composition, followed by a lowering of pressure, and removal of ice by sublimation. In certain cases, lyophilization also involves the removal of bound water molecules through a desorption process. In some embodiments, compositions and vaccine compositions provided herein are spray-dried. Spray drying is a process by which a solution is fed through an atomizer to create a spray, which is thereafter exposed to a heated gas stream to promote rapid evaporation. When sufficient liquid mass has evaporated, the remaining solid material in the droplet forms particles which are then separated from the gas stream (e.g., using a filter or a cyclone). Drying aids in the storage of the compositions and vaccine compositions provided herein at higher temperatures (e.g., greater than 4° C.) as compared to the sub-zero temperatures needed for the storage of existing mRNA vaccines. In some embodiments, dried compositions and lyophilized compositions provided herein comprise (a) a lipid carrier, wherein the lipid carrier is a nanoemulsion comprising: (i) a hydrophobic core; (ii) one or more inorganic nanoparticles; (iii) and one or more lipids; (b) one or more nucleic acids; and (c) at least one cryoprotectant. In some embodiments, the cryoprotectant is selected from the group consisting of: sucrose, maltose, trehalose, mannitol, glucose, and any combinations thereof. Additional examples of cryoprotectants include but are not limited to: dimethyl sulfoxide (DMSO), glycerol, propylene glycol, ethylene glycol, 3-O-methyl-D-glucopyranose (3-OMG), olyethylene glycol (PEG), 1,2-propanediol, acetamide, trehalose, formamide, sugars, proteins, and carbohydrates.

In some embodiments, compositions and methods provided herein comprise at least one cryoprotectant. Exemplary cryoprotectants for inclusion are, but not limited to, sucrose, maltose, trehalose, mannitol, or glucose, and any combinations thereof. In some embodiments, additional or alternative cryoprotectant for inclusion is sorbitol, ribitol, erthritol, threitol, ethylene glycol, or fructose. In some embodiments, additional or alternative cryoprotectant for inclusion is dimethyl sulfoxide (DMSO), glycerol, propylene glycol, ethylene glycol, 3-O-methyl-D-glucopyranose (3-OMG), polyethylene glycol (PEG), 1,2-propanediol, acetamide, trehalose, formamide, sugars, proteins, and carbohydrates. In some embodiments, the cryoprotectant is present at about 1% w/v to at about 20% w/v, preferably about 10% w/v to at about 20% w/v, and more preferably at about 10% w/v. In certain aspects of the disclosure, the cryoprotectant is sucrose. In some aspects of the disclosure, the cryoprotectant is maltose. In some aspects of the disclosure, the cryoprotectant is trehalose. In some aspects of the disclosure, the cryoprotectant is mannitol. In some aspects of the disclosure, the cryoprotectant is glucose. In some embodiments, the cryoprotectant is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the cryoprotectant is present in an amount of about 50 to about 500 mg. In some embodiments, the cryoprotectant is present in an amount of about 200 to about 300 mg. In some embodiments, the cryoprotectant is present in an amount of about 250 mg. In some embodiments, the cryoprotectant is present in amount of a lyophilized composition by weight of at least about 50, 55, 60, 65, 70, 75, 80, 85, 90, 95 or more percent. In some embodiments, the cryoprotectant is present in amount of a lyophilized composition by weight of about 95%. In some embodiments, the cryoprotectant is present in amount of a lyophilized composition by weight of 80 to 98%, 85 to 98%, 90 to 98%, or 94 to 96%. In some embodiments, the cryoprotectant is a sugar. In some embodiments, the sugar is sucrose, maltose, trehalose, mannitol, or glucose. In some embodiments, the sugar is sucrose. In some embodiments, the sucrose is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the sucrose is present in an amount of about 50 to about 500 mg. In some embodiments, the sucrose is present in an amount of about 200 to about 300 mg. In some embodiments, the sucrose is present in an amount of about 250 mg. In some embodiments, the sucrose is present in amount of a lyophilized composition by weight of at least about 50, 55, 60, 65, 70, 75, 80, 85, 90, 95 or more percent. In some embodiments, the sucrose is present in amount of a lyophilized composition by weight of about 95%. In some embodiments, the sucrose is present in amount of a lyophilized composition by weight of 80 to 98%, 85 to 98%, 90 to 98%, or 94 to 96%.

In some embodiments, the cryoprotectant is sucrose. In some embodiments, the cryoprotectant is at a concentration of at least about 0.1% w/v. In some embodiments, the cryoprotectant is at a concentration of about 1% w/v to at about 20% w/v. In some embodiments, the cryoprotectant is at a concentration of about 10% w/v to at about 20% w/v. In some embodiments, the cryoprotectant is at a concentration of about 10% w/v.

In some embodiments, compositions and vaccine compositions provided herein are thermally stable. A composition is considered thermally stable when the composition resists the action of heat or cold and maintains its properties, such as the ability to protect a nucleic acid molecule from degradation at given temperature. In some embodiments, compositions and vaccine compositions provided herein are thermally stable at about 25° C. (° C.) or standard room temperature. In some embodiments, compositions and vaccine compositions provided herein are thermally stable at about 45° C. In some embodiments, compositions and vaccine compositions provided herein are thermally stable at about −20° C. In some embodiments, compositions and vaccine compositions provided herein are thermally stable at about 2° C. to about 8° C. In some embodiments, compositions and vaccine compositions provided herein are thermally stable at a temperature of at least about −80° C., at least about-20° C., at least about 0° C., at least about 2° C., at least about 4° C., at least about 6° C., at least about 8° C., at least about 10° C., at least about 20° C., at least about 25° C., at least about 30° C., at least about 37° C., up to 45° C. In some embodiments, compositions and vaccine compositions provided herein are thermally stable for at least about 5 day, at least about 1 week, at least about 2 weeks, at least about 1 month, up to 3 months. In some embodiments, compositions and vaccine compositions provided herein are stored at a temperature of at least about 4° C. up to 37° C. for at least about 5 day, at least about 1 week, at least about 2 weeks, at least about 1 month, up to 3 months. In some embodiments, compositions and vaccine compositions provided herein are stored at a temperature of at least about 20° C. up to 25° C. for at least about 5 day, at least about 1 week, at least about 2 weeks, at least about 1 month, up to 3 months.

Also provided herein are methods for preparing a lyophilized composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and lyophilizing the formulation to form a lyophilized composition.

Further provided herein are methods for preparing a spray-dried composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and spray drying the formulation to form a spray-dried composition.

Further provided herein are methods for reconstituting a lyophilized composition comprising: obtaining a lipid carrier, wherein the lipid carrier is a nanoemulsion comprising a hydrophobic core, one or more inorganic nanoparticles, and one or more lipids; incorporating one or more nucleic acid into the said lipid carrier to form a lipid carrier-nucleic acid complex; adding at least one cryoprotectant to the lipid carrier-nucleic acid complex to form a formulation; lyophilizing the formulation to form a lyophilized composition; and reconstituting the lyophilized composition in a suitable diluent.

Further provided herein are methods for reconstituting a spray-dried composition comprising: obtaining a lipid carrier, wherein the lipid carrier is a nanoemulsion comprising a hydrophobic core, one or more inorganic nanoparticles, and one or more lipids, incorporating one or more nucleic acid into the said lipid carrier to form a lipid carrier-nucleic acid complex; adding at least one cryoprotectant to the lipid carrier-nucleic acid complex to form a formulation; spray drying the formulation to form a spray-dried composition; and reconstituting the spray-dried composition in a suitable diluent.

### Pharmaceutical Compositions

Provided herein is a suspension comprising a composition provided herein. In some embodiments, suspensions provided herein comprise a plurality of nanoparticles or compositions provided herein. In some embodiments, compositions provided herein are in a suspension, optionally a homogeneous suspension. In some embodiments, compositions provided herein are in an emulsion form.

Also provided herein is a pharmaceutical composition comprising a composition provided herein. In some embodiments, compositions provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

### Dosing

Compositions provided herein may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. Subjects include, without limitation, domesticate or farmed animals (including without limitation pigs, cows, horses, buffalo, pigs, ducks, geese, chicken, turkey, fish) as well as humans. Dosing may be for veterinary or human therapeutic uses. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

### Administration

Provided herein are compositions and pharmaceutical compositions for administering to a subject in need thereof. In some embodiments, pharmaceutical compositions provided here are in a form which allows for compositions provided herein to be administered to a subject.

In some embodiments, the administering is local administration or systemic administration. In some embodiments, a composition described herein is formulated for administration/for use in administration via a subcutaneous, intradermal, intramuscular, inhalation, intravenous, intraperitoneal, intracranial, or intrathecal route. In some embodiments, the administering is every 1, 2, 4, 6, 8, 12, 24, 36, or 48 hours. In some embodiments, the administering is daily, weekly, or monthly. In some embodiments, the administering is repeated at least about every 28 days or 56 days.

In some embodiments, a single dose of a composition provided herein is administered to a subject. In some embodiments, a composition or pharmaceutical composition provided herein is administered to the subject by two doses. In some embodiments, a second dose of a composition or pharmaceutical composition provided herein is administered about 28 days or 56 days after the first dose. In some embodiments, a first dose is administered, and a second dose is administered about 14 days later, or about 21 days later, or about 28 days later, or about 35 days later, or about 42 days later, or about 49 days later, or about 56 days later, or about 63 days later, or about 70 days later, or about 77 days later, or about 84 days later. In some embodiments, the second dose is administered about 10-90 days following administration of the first dose, or about 15-85 days following administration of the first dose, or about 20-80 days following administration of the first dose, or about 25-75 days following administration of the first dose, or about 30-70 days following administration of the first dose, or about 35-65 days following administration of the first dose, or about 40-60 days following administration of the first dose.

In some embodiments, an additional, for example third or more, dose of a composition or pharmaceutical composition provided herein is administered to a subject. In some embodiments, the additional dose is administered about 1 month following administration of the second dose, about 2 months following administration of the second dose, about 3 months following administration of the second dose, about 4 months following administration of the second dose, about 5 months following administration of the second dose, about 6 months following administration of the second dose, about 7 months following administration of the second dose, about 8 months following administration of the second dose, about 9 months following administration of the second dose, about 10 months following administration of the second dose, about 11 months following administration of the second dose, about 12 months following administration of the second dose, about 13 months following administration of the second dose, about 14 months following administration of the second dose, about 15 months following administration of the second dose, about 16 months following administration of the second dose, about 17 months following administration of the second dose, or about 18 months following administration of the second dose.

### Methods

Provided herein are methods of treating or preventing a disease in a subject. In some embodiments, compositions described herein are used for the treatment of cancer. In some embodiments, the cancer is a solid cancer or a blood cancer. In some embodiments, the solid cancer is a carcinoma, a melanoma, or a sarcoma. In some embodiments, the blood cancer is lymphoma or leukemia. In some embodiments, the cancer is a metastatic cancer. In some embodiments, the cancer is a skin cancer. In some embodiments, the skin cancer is a basal cell cancer, a melanoma, a Merkel cell cancer, a squamous cell carcinoma, a cutaneous lymphoma, a Kaposi sarcoma, or a skin adnexal cancer. In some embodiments, the subject has lung cancer. In some embodiments, the lung cancer is a non-small cell lung cancer (NSCLC) or a small cell lung cancer (SCLC). In some embodiments, the NSCLC is an adenocarcinoma, a squamous cell carcinoma, a large cell carcinoma, an adenosquamous carcinoma, or a sarcomatoid carcinoma. In some embodiments, the cancer is a pancreatic cancer. In some embodiments, the pancreatic cancer is a pancreatic adenocarcinoma or a pancreatic exocrine cancer. In some embodiments, the pancreatic cancer is a pancreatic neuroendocrine cancer, an islet cell cancer, or a pancreatic endocrine cancer. In some embodiments, the cancer is a prostate cancer.

Provided herein are personalized treatments for the treatment of a cancer in a subject. The personalized treatment provided herein can be tailored to the expression of a cancer-associated protein provided herein and/or a particular genotype of the subject identified as having a cancer. For example, the subject can be tested for a specific mutation in an oncogenic driver gene (e.g., KRAS G12C or G12D) that is known to cause a specific subtype of cancer (e.g., a NSCLC lung cancer). Oncogenic driver mutations are genetic mutations that are responsible for both the initiation and maintenance of the cancer. In some embodiments, the subject is identified as having a mutation in an oncogenic driver gene or biomarker. Non-limiting examples of oncogenic driver genes/biomarkers and their associated cancer type include: breast cancer: BRCA1, BRCA2, TP53, TTN, FLG, OBSCN, ERBB2, GATA3, FGFRI, CCND1, PIK3CA, CACNA1C, ARHGAP35, ARID5B, BIRC6, CDH1, CTCF, DSPP, HDAC9, KDM5B, MAST1, MEF2A, NCOR2, SETD1A, SXL2, RIDIA, CTNND1, NUP107, CHD8, FANC1, CHD9, CTCF, KEAP1, PCDH18, LAMA2, HDAC9, ARFGEF1, MLLT4, NRK, FOXO3, CDKN2A, MAP3K1, GPS2, ROCK2, RYR2, PGR, STAT6, PIK3CD, CTCF, CDH1, GATA3, AKT1; gastric cancers: ADCY3, BCL6B, CACNAIC, FRMD4A, NID1, ROCK2; pancreatic cancer: ARHGAP35, CACNAIC, GRIA3, PDAC, PALB2, KRAS, CDKN2A, TP53, and SMAD4; lung cancer: EGFR, MET, KRAS, ALK, ALK L1196M, ALK C1156Y, EML4-ALK, ERBB3, ERBB4, VEGFR, NBPF12, NTRK, ROCK2, RYR2, SCAF11, SDK2, STAT6; prostate cancer: SLC45A3, DNAH12, DSPP, KRAS, PCDHIIX, ovarian cancer: DNAH14, PGR, PIK3CD, TTN; colon cancer: LAMA1, PIK3CD, TTN; bladder: RYR2; skin: BRAF V600, NRAS, NRAS Q61L/R, GNAQ, GNA11, AC1, PPP6C, RAC1, PPP6C, STK19.

In some embodiments, methods provided herein comprise modulating an immune response in a subject. In some embodiments, the immune response in the subject is modulated by a method comprising: (a) administering to a subject having a cancer, a composition, wherein the composition comprises: at least one nucleic acid, wherein the at least one nucleic acid comprises a sequence encoding for a plurality of cancer-associated proteins, wherein, prior to the administering, the plurality of cancer-associated proteins are increased in presence compared to non-cancer cells of the subject; or comprise a sequence modification compared to non-cancer cells of the subject; and a plurality of nanoparticles, wherein each nanoparticle comprises a cationic surface, and wherein the at least one nucleic acid is complexed to the cationic surface. Methods provided herein may further comprise obtaining nucleic acid sequence information or amino acid sequence information from a sample comprising cancer cells obtained from the subject. Cancer cells and non-cancer cells can be obtained from the subject by any method, including for example, surgery, biopsy, blood draw, nasal swab, pap test, colonoscopy, urinalysis, and the like. In some embodiments, the cancer cells are circulating cancer cells. Nucleic acid sequence information can be obtained from a sample comprising non-cancer cells from the same subject to serve as a control. The sequence information can be compared between the cancer cell sample and the non-cancer-cell sample to identify somatic mutations present in the cancer cell sequence information, thereby identifying one or more cancer-associated proteins or cancer cell markers in the subject. Sequence information can be obtained from the sample by any method, including but not limited to, e.g., sequencing, PCR, reverse-transcriptase PCR (RT-PCR), proteomics, immunosorbent assays, and RNA-seq. The sequence information can be used to classify immunogenic epitopes with one or more of the following properties: (i) the epitope occurs in a transcript; (ii) the epitope occurs in a protein-coding region; (iii) the epitope introduces a change in an amino acid sequence; and (iv) the epitope is predicted to exhibit MHC binding. These properties are useful for identifying cancer-associated proteins and cancer cell markers to be used in the personalized vaccine composition for administration to the subject. Following the identification of cancer-associated protein epitopes in the cancer cells of the subject, the methods provided herein comprise producing the at least one nucleic acid encoding for a plurality of cancer-associated proteins provided herein; and/or a nucleic acid encoding for an antibody that binds to a cancer-associated protein provided herein.

Provided herein are methods for the personalized treatment of cancer in a subject, the methods comprising: (a) receiving the results of an assay that indicates that the subject has a tumor, wherein the tumor comprises a cancer-associated protein; and (b) administering to the subject a composition, wherein the composition comprises at least one nucleic acid encoding for the cancer-associated protein in (a), thereby treating the cancer in the subject. Further provided herein are method for the personalized treatment of cancer in a subject, the method comprising: (a) receiving the results of an assay that indicates that the subject has a tumor, wherein the tumor comprises a cancer-associated protein; and (b) administering to the subject a composition, wherein the composition comprises at least one nucleic acid encoding for an antibody that specifically binds to the cancer-associated protein in (a), thereby treating the cancer in the subject. In some embodiments, the composition further comprises a nanoparticle described herein. In some embodiments, the nucleic acid further comprises a replicon sequence described herein. In some embodiments, the method provides for reduction in severity, tumor size, tumor volume, or incidence of tumor occurrence in a subject.

In some embodiments, the subject has a tumor comprising a cancer-associated protein selected from the group consisting of: (i) epidermal growth factor receptor (EGFR); (ii) vascular endothelial growth factor (VEGF); (iii) Wilms tumor 1 (WTT); (iv) preferentially expressed antigen of melanoma (PRAME); (v) PR1; (vi) proteinase 3; (vii) elastase; (viii) cathepsin G; (ix) survivin; (x) New York esophagus 1 (NY-Eso-l); (xi) melanoma-associated antigen (MAGE); (xii) tyrosinase; (xiii) glycoprotein 100 (gp100); (xiv) carcinoembryonic antigen (CEA); (xv) mucin; (xvi) fibroblast growth factor (FGF); (xvii) programmed cell death protein (PD-1); (xviii) metastasis tumor antigen (MTA); (xix) human epidermal growth factor receptor 2 (Her2); (xx) Mammaglobin A (SCGB2A2); (xxi) alpha lactalbumin (LALBA); (xxii) cyclin D1 (CCNDT); (xxiii) folate receptor 1 (FOLR1); (xxiv) telomerase (TERT); (xxv) RecQ protein-like (DNA helicase Q1-like) (RECQL); (xxvi) leptin receptor (LEPR); (xxvii) ERBB receptor feedback inhibitor 1(ERRFIl); (xxviii) lysosomal protein transmembrane 4 alpha (LAPTM4A); (xxix) Kirsten rat sarcoma virus (K-Ras); (xxx) S100 protein; (xxxi) a cluster of differentiation (CD) family protein; (xxxii) alphafetoprotein (AFP); (xxxiii) epithelial tumor antigen (ETA); (xxxiv) tumor protein p53; (xxxv) ephrin receptor; (xxxvi) transferrin receptor; (xxxvii) neoglycoprotein; (xxxviii) tumor necrosis factor (TNF)-alpha (a) receptor; (xxxvix) human papillomavirus-E6; (xl) human papillomavirus-E7; (xli) cytokeratin; (xlii) beta-catenin; (xliii) carboxypeptidase M; (xliv) EP4 receptor; (xlv) human milk fat glomeruli antigen; (xlvi) tumor necrosis factor (TNF)-beta ($) receptor; (xlvii) B7-1 protein; (xlviii) B7-2 protein; (xlix) TNF receptor-associated factor 2; (1) Melanoma-associated antigen recognized by T cells 1 (MART-1); and functional fragments thereof. In some embodiments, the subject has a tumor comprising a cancer-associated protein selected from Table 1.

Further provided herein are methods of modulating an immune response in a subject, the methods comprising: administering to a subject having cancer the composition provided herein, or a pharmaceutical composition provided herein. Compositions provided herein can also be administered prophylactically to immunize a subject for a cancer. In some embodiments, compositions described herein are used for prophylactically immunizing a subject for a skin cancer or a lung cancer. In some embodiments, the subject is at risk of developing a cancer described herein. In some embodiments, the administration provides for a reduction in tumor occurrence, size, volume, and/or frequency in a subject compared to a subject having the tumor without the administration.

### Kits

Provided herein is a kit comprising a composition provided herein, a pharmaceutical composition provided herein; and optionally, a delivery system for administration to a subject. Kits described herein may comprise lyophilized reagents and, optionally, a reagent for hydration. Kits described herein may also comprise non-lyophilized reagents. In some embodiments, the kit comprises two or more separate units comprising the lipid carrier and the nucleic acid, respectively.

In some embodiments, the kit comprises a unit that comprises the lipid carrier and the nucleic acid. In some embodiments, the kit further comprises a unit comprising a reagent for hydration of the dried composition. In some embodiments, the reagent for hydration comprises water.

In some embodiments, the kit further comprises one or more surfactants. In some embodiments, a formulation of a composition described herein is prepared in a single container for administration. In some embodiments, a formulation of a composition described herein is prepared two containers for administration, separating the nucleic acid from the nanoparticle carrier. As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some implementations, the containers are RNase free.

In some embodiments, the kit comprises: (a) a lipid carrier, wherein the lipid carrier is a nanoemulsion comprising a hydrophobic core, one or more lipids, and one or more surfactants; and (b) at least one nucleic acid sequence, which comprises a sequence which encodes a sequence capable of expressing an antigen, wherein the antigen is a cancer-associated protein.

### Exemplary Embodiments

Provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the at least one nucleic acid comprises a sequence encoding for a cancer-associated protein. Further provided herein are compositions, wherein the cancer-associated protein is a protein expressed by a melanoma cell. Further provided herein are compositions, wherein the nucleic acid comprises a sequence region that is at least 85% identical to one of SEQ ID NOS: 1-2, 75, 76, 88, or 89. Further provided herein are compositions, wherein the cancer-associated protein sequence or functional variant thereof has an amino acid sequence as set forth in one of SEQ ID NOS: 3-47, 77, 78, 87, 90. Further provided herein are compositions, wherein the nucleic acid is in complex with the lipid carrier. Further provided herein are compositions, wherein the nucleic acid further encodes for an RNA polymerase. Further provided herein are compositions, wherein the RNA polymerase is a Venezuelan equine encephalitis virus (VEEV) RNA polymerase. Further provided herein are compositions, wherein the nucleic acid coding the RNA polymerase comprises the nucleic acid sequence of SEQ ID NO: 71. Further provided herein are compositions, wherein the liquid oil is α-tocopherol, coconut oil, grapeseed oil, lauroyl polyoxylglyceride, mineral oil, monoacylglycerol, palmkernal oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis(azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; $3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9″″′-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis(octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the lipid carrier comprises a hydrophobic core. Further provided herein are compositions, wherein the lipid carrier comprises an inorganic particle. Further provided herein are compositions, wherein the inorganic particle is within the hydrophobic core. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the hydrophilic surfactant is a polysorbate. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the lipid carrier comprises a z-average hydrodynamic diameter ranging from about 40 nm to about 150 nm, with an average polydispersity index ranging from about 0.1 to 0.4. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are in the form of a suspension. Further provided herein are compositions, wherein the compositions are lyophilized.

Further provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the at least one nucleic acid comprises a sequence encoding for an antibody or a functional variant thereof. Further provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the at least one nucleic acid comprises a sequence encoding for a cancer therapeutic antibody or a functional variant thereof. Further provided herein are compositions, wherein the antibody is a cancer therapeutic antibody or a functional variant thereof. Further provided herein are compositions, wherein the cancer therapeutic antibody is an antibody listed in Table 2. Further provided herein are compositions, wherein the cancer therapeutic antibody is antibody is atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab. Further provided herein are compositions, wherein the cancer therapeutic antibody has an amino acid sequence as set forth in any one of SEQ ID NOS: 48-70. Further provided herein are compositions, wherein the nucleic acid is in complex with the lipid carrier. Further provided herein are compositions, wherein the nucleic acid further encodes for an RNA-dependent polymerase. Further provided herein are compositions, wherein the RNA-dependent polymerase is a Venezuelan equine encephalitis virus (VEEV) RNA polymerase. Further provided herein are compositions, wherein the nucleic acid encoding for the RNA-dependent polymerase comprises the nucleic acid sequence of SEQ ID NO: 71. Further provided herein are compositions, wherein the liquid oil is α-tocopherol, coconut oil, grapeseed oil, lauroyl polyoxylglyceride, mineral oil, monoacylglycerol, palmkernal oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis(azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; $3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″,9″″′-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis(octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the lipid carrier comprises a hydrophobic core. Further provided herein are compositions, wherein the lipid carrier comprises an inorganic particle. Further provided herein are compositions, wherein the inorganic particle is within the hydrophobic core. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the hydrophilic surfactant is a polysorbate. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the lipid carrier comprises a z-average hydrodynamic diameter ranging from about 40 nm to about 150 nm, with an average polydispersity index ranging from about 0.1 to 0.4. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are in the form of a suspension. Further provided herein are compositions, wherein the compositions are lyophilized.

Provided herein are compositions, wherein the compositions comprise: a lipid carrier, wherein the lipid carrier comprises: liquid oil; an inorganic nanoparticle, wherein the inorganic nanoparticle comprises iron oxide present in an amount of about 0.2 mg/ml 12 nm iron oxide; and surfactants, wherein the surfactants comprise a cationic lipid; and at least one nucleic acid, wherein the nucleic acid comprises a sequence encoding for a cancer-associated protein sequence or functional variant thereof. Further provided herein are compositions, wherein the lipid carrier further comprises: about 30 mg/mL DOTAP chloride; about 37.5 mg/mL squalene; about 37 mg/ml sorbitan monostearate; about 37 mg/ml polysorbate 80; and about 10 mM sodium citrate. Further provided herein are compositions, wherein the lipid carrier comprises a hydrophobic core. Further provided herein are compositions, wherein the iron oxide comprises oleic acid-coated iron oxide. Further provided herein are compositions, wherein the oleic acid-coated iron oxide nanoparticles are within the hydrophobic core.. Further provided herein are compositions, wherein the liquid oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis(azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; 3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9″″′-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis(octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the nucleic acid is in complex with the lipid carrier. Further provided herein are compositions, wherein the nucleic acid comprises a sequence as set forth in one of SEQ ID NOS: 1-2, 75, 76, 88, or 89. Further provided herein are compositions, wherein the cancer-associated protein sequence or functional variant thereof has an amino acid sequence as set forth in one of SEQ ID NOS: 3-47, 77, 78, 88, 89. Further provided herein are compositions, wherein the nucleic acid further encodes for an RNA-dependent polymerase. Further provided herein are compositions, wherein the RNA-dependent polymerase is a Venezuelan equine encephalitis virus (VEEV) RNA polymerase. Further provided herein are compositions, wherein the nucleic acid coding the RNA-dependent polymerase comprises the nucleic acid sequence of SEQ ID NO: 71. Further provided herein are compositions, wherein the hydrophilic surfactant is a polysorbate. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the lipid carrier comprises a z-average hydrodynamic diameter ranging from about 40 nm to about 150 nm, with an average polydispersity index ranging from about 0.1 to 0.4. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are in the form of a suspension. Further provided herein are compositions, wherein the compositions are lyophilized.

Provided herein are compositions, wherein the compositions comprise: (a) a lipid carrier, wherein the lipid carrier is a nanoemulsion comprising: about 30 mg/mL DOTAP chloride; about 37.5 mg/ml squalene; about 37 mg/ml sorbitan monostearate; about 37 mg/ml polysorbate 80; about 10 mM sodium citrate; and about 0.2 mg Fe/ml 12 nm oleic acid-coated iron oxide nanoparticles; and (b) at least one nucleic acid, wherein the at least one nucleic acid comprises a sequence encoding for a cancer-associated protein sequence or functional variant thereof. Further provided herein are compositions, wherein the compositions further comprise: sucrose, optionally, wherein the sucrose is present in an about of about 50 mg.. Further provided herein are compositions, wherein the nucleic acid is in complex with the lipid carrier. Further provided herein are compositions, wherein the nucleic acid comprises a sequence as set forth in one of SEQ ID NOS: 1-2, 75, 76, 88, or 89. Further provided herein are compositions, wherein the cancer-associated protein sequence or functional variant thereof has an amino acid sequence as set forth in one of SEQ ID NOS: 3-47, 77, 78, 87, or 90. Further provided herein are compositions, wherein the nucleic acid further encodes for an RNA-dependent polymerase. Further provided herein are compositions, wherein the RNA-dependent polymerase is a Venezuelan equine encephalitis virus (VEEV) RNA polymerase. Further provided herein are compositions, wherein the nucleic acid encoding for the RNA-dependent polymerase comprises the nucleic acid sequence of SEQ ID NO: 71. Further provided herein are compositions, wherein the lipid carrier comprises a hydrophobic core. Further provided herein are compositions, wherein the oleic acid-coated iron oxide nanoparticles are within the hydrophobic core. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the lipid carrier comprises a z-average hydrodynamic diameter ranging from about 40 nm to about 150 nm, with an average polydispersity index ranging from about 0.1 to 0.4. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are in the form of a suspension. Further provided herein are compositions, wherein the composition is lyophilized.

Provided herein are compositions, wherein the compositions comprise: (a) a lipid carrier, wherein the lipid carrier is a nanoemulsion comprising: DOTAP chloride present in an amount of about 0.75 mg; squalene present in an amount of about 0.94 mg; sorbitan monostearate present in an amount of about 0.93 mg; polysorbate 80 present in an amount of about 0.93 mg; citric acid monohydrate present in an amount of about 1.05 mg; and oleic acid-coated iron oxide nanoparticles present in an amount of about 0.005 mg; and (b) at least one nucleic acid, wherein the at least one nucleic acid comprises a sequence encoding for a cancer-associated protein sequence or functional variant thereof. Further provided herein are compositions, wherein the nucleic acid is in complex with the lipid carrier. Further provided herein are compositions, wherein the nucleic acid comprises a sequence as set forth in any one of SEQ ID NOS: 1-2, 75, 76, 88, or 89. Further provided herein are compositions, wherein the cancer-associated protein sequence or functional variant thereof has an amino acid sequence as set forth in one of SEQ ID NOS: 3-47, 77, 78, 87, or 90. Further provided herein are compositions, wherein the nucleic acid further encodes for an RNA-dependent polymerase. Further provided herein are compositions, wherein the RNA-dependent polymerase is a Venezuelan equine encephalitis virus (VEEV) RNA polymerase. Further provided herein are compositions, wherein the nucleic acid encoding for the RNA-dependent polymerase comprises the nucleic acid sequence of SEQ ID NO: 71. Further provided herein are compositions, wherein the lipid carrier comprises a hydrophobic core. Further provided herein are compositions, wherein the oleic acid-coated iron oxide nanoparticles are within the hydrophobic core. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the lipid carrier comprises a z-average hydrodynamic diameter ranging from about 40 nm to about 150 nm, with an average polydispersity index ranging from about 0.1 to 0.4. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are in the form of a suspension. Further provided herein are compositions, wherein the compositions are lyophilized.

Provided herein are compositions, wherein the compositions comprise: a first nucleic acid comprising a sequence encoding for an RNA-dependent RNA polymerase; and a second nucleic acid comprising a sequence encoding for a cancer-associated protein sequence or functional variant thereof wherein the cancer-associated protein sequence is least 85% identical to one of SEQ ID NOS: 1-2, 75, 76, 88, or 89. Further provided herein are compositions, wherein the cancer-associated protein sequence comprises a sequence listed in Table 1. Further provided herein are compositions, wherein the cancer-associated protein sequence comprises a TRP-1 tumor associated antigen sequence. Further provided herein are compositions, wherein the first nucleic acid and the second nucleic acid are present on a shared nucleic acid. Further provided herein are compositions, wherein the first nucleic acid and the second nucleic acid are present on separate nucleic acids. Further provided herein are compositions, wherein the RNA-dependent RNA polymerase comprises a VEEV RNA polymerase. Further provided herein are compositions, wherein the compositions further comprise a nanoparticle carrier system. Further provided herein are compositions, wherein the nanoparticle carrier system comprises a cationic lipid and a hydrophobic core. Further provided herein are compositions, wherein the hydrophobic core comprises an inorganic nanoparticle. Further provided herein are compositions, wherein the first nucleic acid and/or the second nucleic acid comprises RNA. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the lipid carrier comprises a z-average hydrodynamic diameter ranging from about 40 nm to about 150 nm, with an average polydispersity index ranging from about 0.1 to 0.4. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are in the form of a suspension. Further provided herein are compositions, wherein the compositions are lyophilized.

Provided herein are compositions, wherein the compositions comprise: a first nucleic acid comprising a sequence encoding for an RNA-dependent RNA polymerase; and a second nucleic acid comprising a sequence encoding for a cancer-associated protein binding antibody or antibody fragment. Further provided herein are compositions, wherein the cancer-associated protein sequence is least 85% identical to a sequence listed in Table 2. Further provided herein are compositions, wherein the first nucleic acid and the second nucleic acid are present on a shared nucleic acid. Further provided herein are compositions, wherein the first nucleic acid and the second nucleic acid are present on separate nucleic acids. Further provided herein are compositions, wherein the RNA-dependent RNA polymerase comprises a VEEV RNA polymerase. Further provided herein are compositions, wherein the compositions further comprise a nanoparticle carrier system. Further provided herein are compositions, wherein the nanoparticle carrier system comprises a cationic lipid and a hydrophobic core. Further provided herein are compositions, wherein the hydrophobic core comprises an inorganic nanoparticle. Further provided herein are compositions, wherein the first nucleic acid and/or the second nucleic acid comprises RNA. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the lipid carrier comprises a z-average hydrodynamic diameter ranging from about 40 nm to about 150 nm, with an average polydispersity index ranging from about 0.1 to 0.4. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are in the form of a suspension. Further provided herein are compositions, wherein the compositions are lyophilized.

Provided herein are compositions, wherein the compositions comprise: (a) a lipid carrier, wherein the lipid carrier is a nanoemulsion comprising a hydrophobic core, optionally one or more inorganic nanoparticles; and one or more lipids; and (b) at least one nucleic acid sequence, wherein the nucleic acid sequence comprises a sequence which encodes a sequence capable of expressing an antigen, wherein the antigen is a cancer-associated protein. Further provided herein are compositions, wherein the nucleic acid is RNA. Further provided herein are compositions, wherein the compositions further comprise a nucleic acid polymerase or a further nucleic acid comprising a sequence which encodes a sequence capable of expressing a nucleic acid polymerase. Further provided herein are compositions, wherein the compositions further comprise an RNA polymerase or a further nucleic acid comprising a sequence which encodes a sequence capable of expressing an RNA polymerase. Further provided herein are compositions, wherein the cancer-associated protein is associated with melanoma. Further provided herein are compositions, wherein the cancer-associated protein sequence comprises a sequence listed in Table 1. Further provided herein are compositions, wherein the cancer-associated protein is MAGE-A1. Further provided herein are compositions, wherein the cancer-associated protein is MAGE-A3. Further provided herein are compositions, wherein the cancer-associated protein is TYRP-1 or TRP-1. Further provided herein are compositions, wherein the hydrophobic core comprises an oil. Further provided herein are compositions, wherein the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, and vitamin E, and a medium chain triglyceride. Further provided herein are compositions, wherein the one or more inorganic nanoparticles is selected from the group consisting of a metal salt, metal oxide, metal hydroxide, metal phosphate, and any combinations thereof. Further provided herein are compositions, wherein the one or more lipids is selected from the group consisting of cationic lipids, anionic lipids, neutral lipids, and any combinations thereof. Further provided herein are compositions, wherein the one or more lipids is a cationic lipid. Further provided herein are compositions, wherein the cationic lipid is selected from the group consisting of 1,2-dioleoyloxy-3-(trimethylammonium)propane (DOTAP); 3β-[N-(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[1-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLinDMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), and any combinations thereof. Further provided herein are compositions, wherein the lipid carrier optionally comprises one or more surfactants. Further provided herein are compositions, wherein the one or more surfactants is selected from the group consisting of hydrophobic surfactant, hydrophilic surfactant, and any combinations thereof. Further provided herein are compositions, wherein the hydrophobic surfactant comprises a sorbitan ester selected from the group consisting of sorbitan monostearate, sorbitan monooleate, and sorbitan trioleate; and the hydrophilic surfactant comprises a polysorbate. Further provided herein are compositions, wherein the lipid carrier have a z-average hydrodynamic diameter ranging from about 40 nm to about 150 nm, with an average polydispersity index ranging from about 0.1 to about 0.4. Further provided herein are compositions, wherein the one or more nucleic acids is incorporated or complexed with the lipid carrier to form a lipid carrier-nucleic acid complex. Further provided herein are compositions, wherein the lipid carrier-RNA complex is formed via non-covalent interactions or via reversible covalent interactions. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the compositions are stable at 2 to 8° C.. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are in the form of a suspension. Further provided herein are compositions, wherein the compositions are lyophilized.

Provided herein are vaccines, wherein the vaccines comprise: (a) a lipid carrier, wherein the lipid carrier is a nanoemulsion comprising a hydrophobic core, optionally one or more inorganic nanoparticles and one or more lipids; and (b) at least one nucleic acid sequence, wherein the nucleic acid sequence comprises a sequence which encodes a sequence capable of expressing an antigen, wherein the antigen is a cancer-associated protein. Further provided herein are vaccines, wherein the nucleic acid is RNA. Further provided herein are vaccines, wherein the vaccines further comprise a nucleic acid polymerase or a further nucleic acid comprising a sequence which encodes a sequence capable of expressing a nucleic acid polymerase. Further provided herein are vaccines, wherein the vaccines further comprise an RNA polymerase or a further nucleic acid comprising a sequence which encodes a sequence capable of expressing an RNA polymerase. Further provided herein are vaccines, wherein the cancer-associated protein is associated with melanoma. Further provided herein are compositions, wherein the cancer-associated protein sequence comprises a sequence listed in Table 1. Further provided herein are vaccines, wherein the cancer-associated protein is MAGE-A1. Further provided herein are vaccines, wherein the cancer-associated protein is MAGE-A3. Further provided herein are vaccines, wherein the cancer-associated protein is TYRP-1 or TRP-1. Further provided herein are vaccines, wherein the hydrophobic core comprises an oil. Further provided herein are vaccines, wherein the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, and vitamin E, and a medium chain triglyceride. Further provided herein are vaccines, wherein the one or more inorganic nanoparticles is selected from the group consisting of a metal salt, metal oxide, metal hydroxide, metal phosphate, and any combinations thereof. Further provided herein are vaccines, wherein the one or more lipids is selected from the group consisting of cationic lipids, anionic lipids, neutral lipids, and any combinations thereof. Further provided herein are vaccines, wherein the one or more lipids is a cationic lipid. Further provided herein are vaccines, wherein the cationic lipid is selected from the group consisting of 1,2-dioleoyloxy-3-(trimethylammonium)propane (DOTAP); 3β-[N-(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[1-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLinDMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), and any combinations thereof. Further provided herein are vaccines, wherein the lipid carrier optionally comprises one or more surfactants. Further provided herein are vaccines, wherein the one or more surfactants is selected from the group consisting of hydrophobic surfactant, hydrophilic surfactant, and any combinations thereof. Further provided herein are vaccines, wherein the hydrophobic surfactant comprises a sorbitan ester selected from the group consisting of sorbitan monostearate, sorbitan monooleate, and sorbitan trioleate; and the hydrophilic surfactant comprises a polysorbate. Further provided herein are vaccines, wherein the lipid carrier have a z-average hydrodynamic diameter ranging from about 40 nm to about 150 nm, with an average polydispersity index ranging from about 0.1 to about 0.4. Further provided herein are vaccines, wherein the one or more nucleic acids is incorporated or complexed with the lipid carrier to form a lipid carrier-nucleic acid complex. Further provided herein are vaccines, wherein the lipid carrier-RNA complex is formed via non-covalent interactions or via reversible covalent interactions. Further provided herein are vaccines, wherein the molar ratio of the lipid carrier to the one or more nucleic acids, characterized by the nitrogen-to-phosphate (N:P) molar ratio, ranges from about 1:1 to about 150:1. Further provided herein are vaccines, wherein the compositions are stable at 2 to 8° C.. Further provided herein are vaccines, wherein the at least one nucleic acid sequence is present in an amount of up to about 100 micrograms (μg). Further provided herein are vaccines, wherein the at least one nucleic acid sequence is present in an amount of up to about 5, about 10, about 25, about 50, or about 100 micrograms (μg). Further provided herein are vaccines, wherein the at least one nucleic acid sequence is present in an amount of up to about 25 μg. Further provided herein are vaccines, wherein the vaccines are in the form of a suspension. Further provided herein are vaccines, wherein the vaccines are lyophilized.

Provided herein are compositions for immunoprotecting a subject, wherein the compositions comprise: a lipid carrier, wherein the lipid carrier is a nanoemulsion comprising a hydrophobic core, one or more inorganic nanoparticles and one or more lipids, and at least one nucleic acid sequence, wherein the nucleic acid sequence comprises a sequence which encodes a sequence capable of expressing an antigen, wherein the antigen is a cancer-associated protein. Further provided herein are compositions, wherein the cancer-associated protein sequence comprises a sequence listed in Table 1. Further provided herein are vaccines, wherein the hydrophobic core comprises an oil. Further provided herein are vaccines, wherein the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, and vitamin E, and a medium chain triglyceride. Further provided herein are vaccines, wherein the one or more inorganic nanoparticles is selected from the group consisting of a metal salt, metal oxide, metal hydroxide, metal phosphate, and any combinations thereof. Further provided herein are vaccines, wherein the one or more lipids is selected from the group consisting of cationic lipids, anionic lipids, neutral lipids, and any combinations thereof. Further provided herein are vaccines, wherein the one or more lipids is a cationic lipid. Further provided herein are vaccines, wherein the cationic lipid is selected from the group consisting of 1,2-dioleoyloxy-3-(trimethylammonium)propane (DOTAP); 3β-[N-(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[1-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLinDMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), and any combinations thereof. Further provided herein are vaccines, wherein the lipid carrier optionally comprises one or more surfactants. Further provided herein are vaccines, wherein the one or more surfactants is selected from the group consisting of hydrophobic surfactant, hydrophilic surfactant, and any combinations thereof. Further provided herein are vaccines, wherein the hydrophobic surfactant comprises a sorbitan ester selected from the group consisting of sorbitan monostearate, sorbitan monooleate, and sorbitan trioleate; and the hydrophilic surfactant comprises a polysorbate. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the lipid carrier comprises a z-average hydrodynamic diameter ranging from about 40 nm to about 150 nm, with an average polydispersity index ranging from about 0.1 to 0.4. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are in the form of a suspension. Further provided herein are compositions, wherein the composition is lyophilized.

Provided herein are dried compositions, wherein the dried compositions comprise: a composition provided herein; and at least one cryoprotectant. Further provided herein are dried compositions, wherein the compositions are lyophilized. Further provided herein are dried compositions, wherein the compositions are spray-dried. Further provided herein are dried compositions, wherein the compositions are thermally stable. Further provided herein are dried compositions, wherein the compositions are thermally stable at about 25° C. Further provided herein are dried compositions, wherein the compositions are thermally stable at about 45° C. Further provided herein are dried compositions, wherein the compositions are thermally stable at about −20° C. Further provided herein are dried compositions, wherein the compositions are thermally stable at about 2° C. to about 8° C. Further provided herein are dried compositions, wherein the compositions are thermally stable for at least 1 week, at least 2 weeks, and/or at least 1 month. Further provided herein are dried compositions, wherein the hydrophobic core comprises an oil. Further provided herein are dried compositions, wherein the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, and vitamin E, and a medium chain triglyceride. Further provided herein are dried compositions, wherein the one or more inorganic nanoparticles is selected from the group consisting of a metal salt, metal oxide, metal hydroxide, metal phosphate, and any combinations thereof. Further provided herein are dried compositions, wherein the one or more lipids is selected from the group consisting of cationic lipids, anionic lipids, neutral lipids, and any combinations thereof. Further provided herein are dried compositions, wherein the one or more lipids is a cationic lipid. Further provided herein are vaccines, wherein the cationic lipid is selected from the group consisting of 1,2-dioleoyloxy-3-(trimethylammonium)propane (DOTAP); 3β-[N-(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[1-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLinDMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), and any combinations thereof. Further provided herein are dried compositions, wherein the lipid carrier optionally comprises one or more surfactants. Further provided herein are dried compositions, wherein the one or more surfactants is selected from the group consisting of hydrophobic surfactant, hydrophilic surfactant, and any combinations thereof. Further provided herein are dried compositions, wherein the hydrophobic surfactant comprises a sorbitan ester selected from the group consisting of sorbitan monostearate, sorbitan monooleate, and sorbitan trioleate; and the hydrophilic surfactant comprises a polysorbate. Further provided herein are dried compositions, wherein the molar ratio of the lipid carrier to the one or more nucleic acids, characterized by the nitrogen-to-phosphate (N:P) molar ratio, ranges from about 1:1 to about 150:1. Further provided herein are dried compositions, wherein the lipid carrier comprises a z-average hydrodynamic diameter ranging from about 40 nm to about 150 nm, with an average polydispersity index ranging from about 0.1 to 0.4. Further provided herein are dried compositions, wherein the at least one nucleic acid sequence is present in an amount of up to about 100 micrograms (μg). Further provided herein are dried compositions, wherein the at least one nucleic acid sequence is present in an amount of up to about 5, about 10, about 25, about 50, or about 100 micrograms (μg). Further provided herein are dried compositions, wherein the at least one nucleic acid sequence is present in an amount of up to about 25 μg.

Provided herein are compositions for prophylaxis of a cancer, the compositions comprising: a sorbitan fatty acid ester, an ethoxylated sorbitan ester, a cationic lipid, an immune stimulant, and at least one RNA encoding an antigen sequence or functional fragment thereof the sorbitan fatty acid ester is sorbitan monostearate. Further provided herein are compositions, wherein the ethoxylated sorbitan ester is polysorbate 80. Further provided herein are compositions, wherein the cationic lipid is DOTAP. Further provided herein are compositions, wherein the immune stimulant is squalene. Further provided herein are compositions, wherein the RNA encodes a cancer-associated protein. Further provided herein are compositions, wherein the immune stimulant decreases the total amount of protein produced, but increases the immune response to the vaccine. Further provided herein are compositions, wherein n the immune stimulant increases the total amount of protein, produced, but decreases the immune response to the vaccine. Further provided herein are compositions, wherein the immune stimulant is Miglyol 810 or Miglyol 812. Further provided herein are compositions, wherein the composition comprises squalene and no solid particles. Further provided herein are compositions, wherein the ratio of the esters yields a Hydrophilic-Lipophilic Balance between 8 and 11. Further provided herein are compositions, wherein the particle size is between 30 and 200 nanometers. Further provided herein are compositions, wherein the N to P ratio is between 5 and 35. Further provided herein are compositions, wherein the antigen sequence comprises a sequence listed in Table 1. Further provided herein are compositions, wherein the compositions are in the form of a suspension. Further provided herein are compositions, wherein the composition is lyophilized.

Provided herein are compositions for prophylaxis of a cancer, the compositions comprising: sorbitan monostearate (e.g., SPAN® 60), polysorbate 80 (e.g., TWEEN® 80), DOTAP, an immune stimulant, and at least one RNA encoding an antigen sequence or functional fragment thereof. Further provided herein are compositions, wherein the immune stimulant decreases the total amount of protein produced, but increases the immune response to the vaccine. Further provided herein are compositions, wherein n the immune stimulant increases the total amount of protein, produced, but decreases the immune response to the vaccine. Further provided herein are compositions, wherein the immune stimulant is Miglyol 810 or Miglyol 812. Further provided herein are compositions, wherein the composition comprises squalene and no solid particles. Further provided herein are compositions, wherein the ratio of the esters yields a Hydrophilic-Lipophilic Balance between 8 and 11. Further provided herein are compositions, wherein the particle size is between 30 and 200 nanometers. Further provided herein are compositions, wherein the N to P ratio is between 5 and 35. Further provided herein are compositions, wherein the antigen sequence comprises a sequence listed in Table 1. Further provided herein are compositions, wherein the compositions are in the form of a suspension. Further provided herein are compositions, wherein the composition is lyophilized. Further provided herein are pharmaceutical compositions, wherein the pharmaceutical compositions comprise: a composition provided herein; and a pharmaceutically acceptable excipient. Further provided herein are pharmaceutical compositions, wherein the pharmaceutical compositions comprise: (a) a lipid carrier, wherein the lipid carrier is a nanoemulsion comprising a hydrophobic core, optionally one or more inorganic nanoparticles and one or more lipids; and (b) at least one nucleic acid sequence, wherein the nucleic acid sequence comprises a sequence which encodes a sequence capable of expressing an antigen, wherein the antigen is a cancer-associated protein.

Provided herein are kits, wherein the kits comprise a composition provided herein.

Provided herein are methods of generating an immune response in a subject, the methods comprise: administering to a subject a composition provided herein, thereby generating an immune response to a cancer-associated protein. Further provided herein are methods of generating an immune response in a subject, wherein the methods comprise: administering to a subject: (a) a lipid carrier, wherein the lipid carrier is a nanoemulsion comprising a hydrophobic core, optionally one or more inorganic nanoparticles and one or more lipids; and (b) at least one nucleic acid sequence, wherein the nucleic acid sequence comprises a sequence which encodes a sequence capable of expressing an antigen, wherein the antigen is a cancer-associated protein. Further provided herein are methods, wherein the composition is administered to the subject by two doses. Further provided herein are methods, wherein the second dose is administered at about 28 days after the first dose. Further provided herein are methods, wherein the methods further comprise: administering a third dose of the composition to said subject. Further provided herein are methods, wherein 5 μg of the composition is administered to the subject. Further provided herein are methods, wherein 10 μg of the composition is administered to said subject. Further provided herein are methods, wherein 25 μg of the composition is administered to said subject. Further provided herein are methods, wherein the composition is administered via intramuscular injection, intranasal administration, oral administration, subcutaneous administration, intratumoral administration, intrathecal administration, or intravenous injection. Further provided herein are methods, wherein the subject is a human. Further provided herein are methods, wherein the subject has, is at risk for, or is suspected of having a cancer. Further provided herein are methods, wherein the subject has a solid tumor or a blood cancer. Further provided herein are methods, wherein the solid tumor is a carcinoma, a melanoma, or a sarcoma. Further provided herein are methods, wherein the blood cancer is lymphoma or leukemia. Further provided herein are methods, wherein the subject has, is at risk for, or is suspected of having a skin cancer. Further provided herein are methods, wherein the skin cancer is a basal cell cancer, a melanoma, a Merkel cell cancer, a squamous cell carcinoma, a cutaneous lymphoma, a Kaposi sarcoma, or a skin adnexal cancer.

Further provided herein are methods of prophylactically immunizing a subject for a cancer, the methods comprise: administering to a subject a composition provided herein, thereby immunizing the subject to a cancer expressing a cancer-associated protein. Further provided herein are methods, wherein the subject is at risk for developing a skin cancer. Further provided herein are methods, wherein 5 μg of the composition is administered to the subject. Further provided herein are methods, wherein 10 μg of the composition is administered to said subject. Further provided herein are methods, wherein 25 μg of the composition is administered to said subject. Further provided herein are methods, wherein the composition is administered via intramuscular injection, intranasal administration, oral administration, subcutaneous administration, intratumoral administration, intrathecal administration, or intravenous injection. Further provided herein are methods, wherein the subject is a human. Further provided herein are methods, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are methods, wherein the cancer expresses a cancer-associated protein, wherein the cancer-associated protein is a TRP-1 protein.

Further provided herein are methods of reducing the severity of a cancer, wherein the methods comprise: administering a composition comprising a lipid carrier, wherein the lipid carrier is a nanoemulsion comprising a hydrophobic core, optionally one or more inorganic nanoparticles and one or more lipids, and at least one nucleic acid sequence, wherein the nucleic acid sequence comprises a sequence which encodes a sequence capable of expressing an antigen, wherein the antigen is a cancer-associated protein.

Further provided herein are methods of immunoprotecting a subject, wherein the methods comprise: administering to the subject a lipid carrier, wherein the lipid carrier is a nanoemulsion comprising a hydrophobic core, one or more inorganic nanoparticles and one or more lipids, at least one nucleic acid sequence, wherein the nucleic acid sequence comprises a sequence which encodes a sequence capable of expressing an antigen, wherein the antigen is a cancer-associated protein.

Provided herein are methods of generating an immune response in a subject, wherein the methods comprise: administering to said subject a composition comprising a lipid carrier, wherein the lipid carrier is a nanoemulsion comprising a hydrophobic core, one or more inorganic nanoparticles and one or more lipids, and at least one nucleic acid sequence, wherein the nucleic acid sequence comprises a sequence which encodes a sequence capable of expressing an antigen, wherein the antigen is a cancer-associated protein. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the lipid carrier comprises a z-average hydrodynamic diameter ranging from about 40 nm to about 150 nm, with an average polydispersity index ranging from about 0.1 to 0.4. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are in the form of a suspension. Further provided herein are compositions, wherein the composition is lyophilized.

Further provided herein are methods for preparing a lyophilized composition, wherein the methods comprising: obtaining a lipid carrier, wherein the lipid carrier is a nanoemulsion comprising a hydrophobic core, one or more inorganic nanoparticles and one or more lipids, incorporating at least one nucleic acid into the lipid carrier to form a lipid carrier-nucleic acid complex, wherein the nucleic acid has a sequence comprising an antigen sequence as set forth in one of SEQ ID NOS: 1-2, 75, 76, 88, or 89; adding at least one cryoprotectant to the lipid carrier-nucleic acid complex to form a formulation, and lyophilizing the formulation to form a lyophilized composition.

Further provided herein are methods for preparing a spray-dried composition, wherein the methods comprise: obtaining a lipid carrier, wherein the lipid carrier is a nanoemulsion comprising a hydrophobic core, one or more inorganic nanoparticles and one or more lipids, incorporating at least one nucleic acid into the lipid carrier to form a lipid carrier-nucleic acid complex, wherein the nucleic acid has a sequence comprising an antigen sequence as set forth in one of SEQ ID NOS: 1-2, 75, 76, 88, or 89; adding at least one cryoprotectant to the lipid carrier-nucleic acid complex to form a formulation, and spray drying the formulation to form a spray-dried composition.

The present invention also relates to a method for reconstituting a lyophilized composition comprising obtaining a lipid carrier, wherein the lipid carrier is a nanoemulsion comprising a hydrophobic core, one or more inorganic nanoparticles, and one or more lipids, incorporating at least one nucleic acid into the said lipid carrier to form a lipid carrier-nucleic acid complex, wherein the nucleic acid has a sequence comprising an antigen sequence as set forth in one of SEQ ID NOS: 1-2, 75 or 76; adding at least one cryoprotectant to the lipid carrier-nucleic acid complex to form a formulation, lyophilizing the formulation to form a lyophilized composition, and reconstituting the lyophilized composition in a suitable diluent. Further provided herein are methods, wherein the diluent is aqueous. Further provided herein are methods, wherein the diluent is water. Further provided herein are methods, wherein the lyophilized compositions are thermally stable. Further provided herein are methods, wherein the lyophilized compositions are thermally stable at temperatures up to about 25° C. Further provided herein are methods, wherein the lyophilized compositions are thermally stable at temperatures up to about 45° C. Further provided herein are methods, wherein the lyophilized compositions are thermally stable at temperatures down to about −20° C. Further provided herein are methods, wherein the lyophilized compositions are thermally stable at temperatures ranging from about 2° C. to about 8° C. Further provided herein are methods, wherein the lyophilized compositions are thermally stable for at least 1 week, at least 2 weeks, and/or at least 1 month. Further provided herein are methods, wherein the hydrophobic core comprises an oil. Further provided herein are methods, wherein the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, vitamin E, medium chain triglyceride, dihydroisosqualene (DHIS), famesene and squalane. Further provided herein are methods, wherein the one or more inorganic nanoparticles is selected from the group consisting of metal salts, metal oxides, metal hydroxides, metal phosphates, metalloids and any combinations thereof. Further provided herein are methods, wherein the one or more lipids is selected from the group consisting of cationic lipids, anionic lipids, neutral lipids, and any combinations thereof. Further provided herein are methods, wherein the one or more lipids is a cationic lipid. Further provided herein are methods, wherein the cationic lipid is selected from the group consisting of 1,2-dioleoyloxy-3-(trimethylammonium)propane (DOTAP); 3β-[N-(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[1-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLin-DMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), 1,2-dioleoyl-sn-glycero-3-phosphoethanolamine (DOPE); N-decyl-N,N-dimethyldecan-1-aminium bromide (DDAB); 2,3-dioleyloxy-N-[2-(sperminecarboxamido)ethyl]-N,N-dimethyl-1-propanaminium trifluoroacetate (DOSPA); ethylphosphatidylcholine (ePC); and any combinations thereof. Further provided herein are methods, wherein the lipid carrier optionally comprises one or more surfactant. Further provided herein are methods, wherein the one or more surfactant is selected from the group consisting of hydrophobic surfactant, hydrophilic surfactant, and any combinations thereof. Further provided herein are methods, wherein the hydrophobic surfactant comprises a sorbitan ester selected from the group consisting of SPAN® 20, SPAN® 40, SPAN® 60, SPAN® 65, SPAN®80 and SPAN® 85; and the hydrophilic surfactant comprises a polysorbate. Further provided herein are methods, wherein the lipid carrier has a z-average hydrodynamic diameter ranging from about 40 nm to about 150 nm, with an average polydispersity index ranging from about 0.1 to about 0.4. Further provided herein are methods, wherein the one or more nucleic acid is an RNA. Further provided herein are methods, wherein the RNA is a self-replicating RNA. Further provided herein are methods, wherein the one or more nucleic acid comprises a sequence which encodes an antigen, wherein the antigen is derived from a virus. Further provided herein are methods, wherein the virus is an oncovirus. Further provided herein are methods, wherein the RNA encodes an amino acid sequence which is at least 80% identical to the amino acid sequence of MAGE-A1 or MAGE-A3. Further provided herein are methods, wherein the RNA encodes an amino acid sequence which is at least 80% identical to the amino acid sequence of TRYP-1 or TRP-1. Further provided herein are methods, wherein the one or more nucleic acid is incorporated or complexed with the lipid carrier to form a lipid carrier-nucleic acid complex. Further provided herein are methods, wherein the lipid carrier-nucleic acid complex is formed via non-covalent interactions or via reversible covalent interactions. Further provided herein are methods, wherein the at least one cryoprotectant is selected from the group consisting of sucrose, maltose, trehalose, mannitol, glucose, and any combinations thereof. Further provided herein are methods, wherein the at least one cryoprotectant is sucrose. Further provided herein are methods, wherein the at least one cryoprotectant is at about 1% w/v to at about 20% w/v. Further provided herein are methods, wherein the at least one cryoprotectant is at about 10% w/v to at about 20% w/v. Further provided herein are methods, wherein the at least one cryoprotectant is at about 10% w/v.

Further provided herein are methods for reconstituting a spray-dried composition, wherein the methods comprise: obtaining a lipid carrier, wherein the lipid carrier is a nanoemulsion comprising a hydrophobic core, one or more inorganic nanoparticles, and one or more lipids, incorporating at least one nucleic acid into the said lipid carrier to form a lipid carrier-nucleic acid complex, wherein the nucleic acid has a sequence comprising an antigen sequence as set forth in one of SEQ ID NOS: 1-2, 75 or 76; adding at least one cryoprotectant to the lipid carrier-nucleic acid complex to form a formulation, spray drying the formulation to form a spray-dried composition, and reconstituting the spray-dried composition in a suitable diluent. Further provided herein are methods, wherein the diluent is aqueous. Further provided herein are methods, wherein the diluent is water. Further provided herein are methods, wherein the spray-dried compositions are thermally stable. Further provided herein are methods, wherein the spray-dried compositions are thermally stable at temperatures up to about 25° C. Further provided herein are methods, wherein the spray-dried compositions are thermally stable at temperatures up to about 45° C. Further provided herein are methods, wherein the spray-dried compositions are thermally stable at temperatures down to about −20° C. Further provided herein are methods, wherein the spray-dried compositions are thermally stable at temperatures ranging from about 2° C. to about 8° C. Further provided herein are methods, wherein the spray-dried compositions are thermally stable for at least 1 week, at least 2 weeks, and/or at least 1 month. Further provided herein are methods, wherein the hydrophobic core comprises an oil. Further provided herein are methods, wherein the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, vitamin E, medium chain triglyceride, dihydroisosqualene (DHIS), farnesene and squalane. Further provided herein are methods, wherein the one or more inorganic nanoparticles is selected from the group consisting of metal salts, metal oxides, metal hydroxides, metal phosphates, metalloids and any combinations thereof. Further provided herein are methods, wherein the one or more lipids is selected from the group consisting of cationic lipids, anionic lipids, neutral lipids, and any combinations thereof. Further provided herein are methods, wherein the one or more lipids is a cationic lipid. Further provided herein are methods, wherein the cationic lipid is selected from the group consisting of 1,2-dioleoyloxy-3-(trimethylammonium)propane (DOTAP); 3β-[N-(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[1-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLin-DMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), 1,2-dioleoyl-sn-glycero-3-phosphoethanolamine (DOPE); N-decyl-N,N-dimethyldecan-1-aminium bromide (DDAB); 2,3-dioleyloxy-N-[2-(sperminecarboxamido)ethyl]-N,N-dimethyl-1-propanaminium trifluoroacetate (DOSPA); ethylphosphatidylcholine (ePC); and any combinations thereof. Further provided herein are methods, wherein the lipid carrier optionally comprises one or more surfactant. Further provided herein are methods, wherein the one or more surfactant is selected from the group consisting of hydrophobic surfactant, hydrophilic surfactant, and any combinations thereof. Further provided herein are methods, wherein the hydrophobic surfactant comprises a sorbitan ester selected from the group consisting of SPAN® 20, SPAN® 40, SPAN® 60, SPAN® 65, SPAN® 80 and SPAN® 85; and the hydrophilic surfactant comprises a polysorbate. Further provided herein are methods, wherein the lipid carrier has a z-average hydrodynamic diameter ranging from about 40 nm to about 150 nm, with an average polydispersity index ranging from about 0.1 to about 0.4. Further provided herein are methods, wherein the one or more nucleic acid is an RNA. Further provided herein are methods, wherein the RNA is a self-replicating RNA. Further provided herein are methods, wherein the one or more nucleic acid comprises a sequence which encodes an antigen, wherein the antigen is derived from a virus. Further provided herein are methods, wherein the virus is an oncovirus. Further provided herein are methods, wherein the RNA encodes an amino acid sequence which is at least 80% identical to the amino acid sequence of MAGE-A1 or MAGE-A3. Further provided herein are methods, wherein the RNA encodes an amino acid sequence which is at least 80% identical to the amino acid sequence of TRYP-1 or TRP-1. Further provided herein are methods, wherein the one or more nucleic acid is incorporated or complexed with the lipid carrier to form a lipid carrier-nucleic acid complex. Further provided herein are methods, wherein the lipid carrier-nucleic acid complex is formed via non-covalent interactions or via reversible covalent interactions. Further provided herein are methods, wherein the at least one cryoprotectant is selected from the group consisting of sucrose, maltose, trehalose, mannitol, glucose, and any combinations thereof. Further provided herein are methods, wherein the at least one cryoprotectant is sucrose. Further provided herein are methods, wherein the at least one cryoprotectant is at about 1% w/v to at about 20% w/v. Further provided herein are methods, wherein the at least one cryoprotectant is at about 10% w/v to at about 20% w/v. Further provided herein are methods, wherein the at least one cryoprotectant is at about 10% w/v.

Provided herein are compositions, wherein the compositions comprise: at least one nucleic acid, wherein the at least one nucleic acid comprises a sequence encoding for: a plurality of cancer-associated proteins or functional fragments thereof; and an RNA polymerase complex region; and a plurality of nanoparticles, wherein each nanoparticle comprises a cationic surface, and wherein the at least one nucleic acid is complexed to the cationic surface. Further provided herein are compositions, wherein the at least one nucleic acid is RNA or DNA. Further provided herein are compositions, wherein the plurality of cancer-associated proteins are expressed by one or more cancer cells of a subject. Further provided herein are compositions, wherein the subject has a solid tumor or a blood cancer. Further provided herein are compositions, wherein the solid tumor is a carcinoma, a melanoma, or a sarcoma. Further provided herein are compositions, wherein the blood cancer is lymphoma or leukemia. Further provided herein are compositions, wherein the subject has a skin cancer. Further provided herein are compositions, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are compositions, wherein the subject has a lung cancer. Further provided herein are compositions, wherein the lung cancer is a non-small cell lung cancer (NSCLC) or a small cell lung cancer (SCLC). Further provided herein are compositions, wherein the NSCLC is an adenocarcinoma, a squamous cell carcinoma, a large cell carcinoma, an adenosquamous carcinoma, or a sarcomatoid carcinoma. Further provided herein are compositions, the subject has a pancreatic cancer. Further provided herein are compositions, wherein the pancreatic cancer is a pancreatic adenocarcinoma, a pancreatic exocrine cancer, a pancreatic neuroendocrine cancer, an islet cell cancer, or a pancreatic endocrine cancer. Further provided herein are compositions, wherein the subject has a metastatic cancer. Further provided herein are compositions, wherein the at least one nucleic acid encodes for two or more cancer-associated proteins, wherein the two or more cancer-associated proteins are selected from:(i) epidermal growth factor receptor (EGFR); (ii) vascular endothelial growth factor (VEGF); (iii) Wilms tumor 1 (WT1); (iv) preferentially expressed antigen of melanoma (PRAME); (v) PR1; (vi) proteinase 3; (vii) elastase; (viii) cathepsin G; (ix) survivin; (x) New York esophagus 1 (NY-Eso-l); (xi) melanoma-associated antigen (MAGE); (xii) tyrosinase; (xiii) glycoprotein 100 (gp100); (xiv) carcinoembryonic antigen (CEA); (xv) mucin; (xvi) fibroblast growth factor (FGF); (xvii) programmed cell death protein (PD-1); (xviii) metastasis tumor antigen (MTA); (xix) human epidermal growth factor receptor 2 (Her2); (xx) Mammaglobin A (SCGB2A2); (xxi) alpha lactalbumin (LALBA); (xxii) cyclin D1 (CCND1); (xxiii) folate receptor 1 (FOLR1); (xxiv) telomerase (TERT); (xxv) RecQ protein-like (DNA helicase Q1-like) (RECQL); (xxvi) leptin receptor (LEPR); (xxvii) ERBB receptor feedback inhibitor 1(ERRFI1); (xxviii) lysosomal protein transmembrane 4 alpha (LAPTM4A); (xxix) Kirsten rat sarcoma virus (K-Ras); (xxx) S100 protein; (xxxi) a cluster of differentiation (CD) family protein; (xxxii) alphafetoprotein (AFP); (xxxiii) epithelial tumor antigen (ETA); (xxxiv) tumor protein p53; (xxxv) ephrin receptor; (xxxvi) transferrin receptor; (xxxvii) neoglycoprotein; (xxxviii) tumor necrosis factor (TNF)-alpha (a) receptor; (xxxvix) human papillomavirus-E6; (xl) human papillomavirus-E7; (xli) cytokeratin; (xlii) beta-catenin; (xliii) carboxypeptidase M; (xliv) EP4 receptor; (xlv) human milk fat glomeruli antigen; (xlvi) tumor necrosis factor (TNF)-beta (P) receptor; (xlvii) B7-1 protein; (xlviii) B7-2 protein; (xlix) TNF receptor-associated factor 2; (1) Melanoma-associated antigen recognized by T cells 1 (MART-1); and functional fragments thereof. Further provided herein are compositions, wherein the MAGE is MAGE-A1, MAGE-A3, MART-1/Melan-A, MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-All, or MAGE-A12. Further provided herein are compositions, the cluster of differentiation family protein is CD5, CD19, CD20, CD22, CD23, CD25, CD27, CD30, CD33, CD36, CD46, CD52, CD79a, CD79b, CD123, or CD317. Further provided herein are compositions, the RNA polymerase complex region is downstream of a subgenomic promoter from an alphavirus. Further provided herein are compositions, wherein RNA polymerase complex region encodes for an RNA-dependent RNA polymerase. Further provided herein are compositions, the RNA-dependent RNA polymerase is Venezuelan equine encephalitis virus (VEEV) RNA polymerase. Further provided herein are compositions, wherein the cationic surface comprises a cationic lipid. Further provided herein are compositions, wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis(azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; $3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9″″′-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis(octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein each nanoparticle further comprises a hydrophobic core. Further provided herein are compositions, wherein the hydrophobic core comprises an oil. Further provided herein are compositions, wherein the oil is in liquid phase. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein each nanoparticle comprises a cationic lipid and an oil. Further provided herein are compositions, wherein each nanoparticle further comprises a surfactant. Further provided herein are compositions, wherein the surfactant is polysorbate, a phosphorous-terminated surfactant, a carboxylate-terminated surfactant, a sulfate-terminated surfactant, an amine-terminated surfactant, trioctylphosphine oxide (TOPO), or distearyl phosphatidic acid (DSPA). Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension.

Provided herein are compositions, wherein the compositions comprise: a plurality of nucleic acids, wherein the plurality of nucleic acids comprises sequence encoding separately for: a plurality of cancer-associated proteins; and an RNA polymerase complex region; and a plurality of nanoparticles, wherein each nanoparticle comprises a cationic surface, and wherein the at least one nucleic acid is complexed to the cationic surface. Further provided herein are compositions, wherein the at least one nucleic acid is RNA or DNA. Further provided herein are compositions, wherein the plurality of cancer-associated proteins are expressed by one or more cancer cells of a subject. Further provided herein are compositions, wherein the subject has a solid tumor or a blood cancer. Further provided herein are compositions, wherein the solid tumor is a carcinoma, a melanoma, or a sarcoma. Further provided herein are compositions, wherein the blood cancer is lymphoma or leukemia. Further provided herein are compositions, wherein the subject has a skin cancer. Further provided herein are compositions, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are compositions, wherein the subject has a lung cancer. Further provided herein are compositions, wherein the lung cancer is a non-small cell lung cancer (NSCLC) or a small cell lung cancer (SCLC). Further provided herein are compositions, wherein the NSCLC is an adenocarcinoma, a squamous cell carcinoma, a large cell carcinoma, an adenosquamous carcinoma, or a sarcomatoid carcinoma. Further provided herein are compositions, the subject has a pancreatic cancer. Further provided herein are compositions, wherein the pancreatic cancer is a pancreatic adenocarcinoma, a pancreatic exocrine cancer, a pancreatic neuroendocrine cancer, an islet cell cancer, or a pancreatic endocrine cancer. Further provided herein are compositions, wherein the subject has a metastatic cancer. Further provided herein are compositions, wherein the at least one nucleic acid encodes for two or more cancer-associated proteins, wherein the two or more cancer-associated proteins are selected from:(i) epidermal growth factor receptor (EGFR); (ii) vascular endothelial growth factor (VEGF); (iii) Wilms tumor 1 (WT1); (iv) preferentially expressed antigen of melanoma (PRAME); (v) PR1; (vi) proteinase 3; (vii) elastase; (viii) cathepsin G; (ix) survivin; (x) New York esophagus 1 (NY-Eso-l); (xi) melanoma-associated antigen (MAGE); (xii) tyrosinase; (xiii) glycoprotein 100 (gp100); (xiv) carcinoembryonic antigen (CEA); (xv) mucin; (xvi) fibroblast growth factor (FGF); (xvii) programmed cell death protein (PD-1); (xviii) metastasis tumor antigen (MTA); (xix) human epidermal growth factor receptor 2 (Her2); (xx) Mammaglobin A (SCGB2A2); (xxi) alpha lactalbumin (LALBA); (xxii) cyclin D1 (CCND1); (xxiii) folate receptor 1 (FOLR1); (xxiv) telomerase (TERT); (xxv) RecQ protein-like (DNA helicase Q1-like) (RECQL); (xxvi) leptin receptor (LEPR); (xxvii) ERBB receptor feedback inhibitor 1(ERRFI1); (xxviii) lysosomal protein transmembrane 4 alpha (LAPTM4A); (xxix) Kirsten rat sarcoma virus (K-Ras); (xxx) S100 protein; (xxxi) a cluster of differentiation (CD) family protein; (xxxii) alphafetoprotein (AFP); (xxxiii) epithelial tumor antigen (ETA); (xxxiv) tumor protein p53; (xxxv) ephrin receptor; (xxxvi) transferrin receptor; (xxxvii) neoglycoprotein; (xxxviii) tumor necrosis factor (TNF)-alpha (α) receptor; (xxxvix) human papillomavirus-E6; (xl) human papillomavirus-E7; (xli) cytokeratin; (xlii) beta-catenin; (xliii) carboxypeptidase M; (xliv) EP4 receptor; (xlv) human milk fat glomeruli antigen; (xlvi) tumor necrosis factor (TNF)-beta (P) receptor; (xlvii) B7-1 protein; (xlviii) B7-2 protein; (xlix) TNF receptor-associated factor 2; (1) Melanoma-associated antigen recognized by T cells 1 (MART-1); and functional fragments thereof. Further provided herein are compositions, wherein the MAGE is MAGE-A1, MAGE-A3, MART-1/Melan-A, MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-All, or MAGE-A12. Further provided herein are compositions, the cluster of differentiation family protein is CD5, CD19, CD20, CD22, CD23, CD25, CD27, CD30, CD33, CD36, CD46, CD52, CD79a, CD79b, CD123, or CD317. Further provided herein are compositions, the RNA polymerase complex region is downstream of a subgenomic promoter from an alphavirus. Further provided herein are compositions, wherein RNA polymerase complex region encodes for an RNA-dependent RNA polymerase. Further provided herein are compositions, the RNA-dependent RNA polymerase is Venezuelan equine encephalitis virus (VEEV) RNA polymerase. Further provided herein are compositions, wherein the cationic surface comprises a cationic lipid. Further provided herein are compositions, wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis(azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; $3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″9′91 9″″′″-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,127,12″Z,12″′Z)-tetrakis(octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein each nanoparticle further comprises a hydrophobic core. Further provided herein are compositions, wherein the hydrophobic core comprises an oil. Further provided herein are compositions, wherein the oil is in liquid phase. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein each nanoparticle comprises a cationic lipid and an oil. Further provided herein are compositions, wherein each nanoparticle further comprises a surfactant. Further provided herein are compositions, wherein the surfactant is polysorbate, a phosphorous-terminated surfactant, a carboxylate-terminated surfactant, a sulfate-terminated surfactant, an amine-terminated surfactant, trioctylphosphine oxide (TOPO), or distearyl phosphatidic acid (DSPA). Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension.

Provided herein are compositions, wherein the compositions comprise: at least one nucleic acid, wherein the at least one nucleic acid comprises a sequence encoding for: an antibody or a functional fragment thereof; and an RNA polymerase complex region; and a plurality of nanoparticles, wherein each nanoparticle comprises a cationic surface, and wherein the at least one nucleic acid is complexed to the cationic surface. Further provided herein are compositions, wherein the an antibody or a functional fragment thereof is a cancer therapeutic antibody. Further provided herein are compositions, wherein the antibody is a monoclonal antibody. Further provided herein are compositions, wherein the antibody, the antibody fragment, or the functional fragment thereof is a murine antibody, a camelid antibody, a humanized antibody, or a fully human antibody. Further provided herein are compositions, wherein the antibody is an immunoglobulin (Ig) molecule or a functional fragment thereof. Further provided herein are compositions, wherein the immunoglobulin molecule is an IgG, IgE, IgM, IgD, IgA, or an IgY isotype immunoglobulin molecule or a functional fragment thereof. Further provided herein are compositions, wherein the immunoglobulin molecule of the functional fragment comprises at least a fragment of an IgG1, an IgG2, an IgG3, an IgG4, an IgGA1, or an IgGA2 subclass immunoglobulin molecule. Further provided herein are compositions, wherein the antibody or the functional fragment thereof is recombinant, chimeric, or multivalent. Further provided herein are compositions, wherein the antibody or the functional fragment thereof is a bispecific antibody, a trispecific antibody, a multispecific antibody, or a functional fragment thereof. Further provided herein are compositions, wherein the antibody or the functional fragment is an antigen-binding fragment (Fab), and Fab2a F(ab′), a F(ab′)2, an dAb, an Fe, a Fv, a disulfide linked Fv, a scFv, a tandem scFv, a free LC, a half antibody, a single domain antibody (dAb), a diabody, or a nanobody. Further provided herein are compositions, wherein the antibody or functional fragment thereof specifically binds to a cancer-associated protein or a microbial antigen. Further provided herein are compositions, wherein the antibody or functional fragment thereof is atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab. Further provided herein are compositions, wherein the at least one nucleic acid is RNA or DNA. Further provided herein are compositions, wherein the plurality of cancer-associated proteins are expressed by one or more cancer cells of a subject. Further provided herein are compositions, wherein the subject has a solid tumor or a blood cancer. Further provided herein are compositions, wherein the solid tumor is a carcinoma, a melanoma, or a sarcoma. Further provided herein are compositions, wherein the blood cancer is lymphoma or leukemia. Further provided herein are compositions, wherein the subject has a skin cancer. Further provided herein are compositions, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are compositions, wherein the subject has a lung cancer. Further provided herein are compositions, wherein the lung cancer is a non-small cell lung cancer (NSCLC) or a small cell lung cancer (SCLC). Further provided herein are compositions, wherein the NSCLC is an adenocarcinoma, a squamous cell carcinoma, a large cell carcinoma, an adenosquamous carcinoma, or a sarcomatoid carcinoma. Further provided herein are compositions, the subject has a pancreatic cancer. Further provided herein are compositions, wherein the pancreatic cancer is a pancreatic adenocarcinoma, a pancreatic exocrine cancer, a pancreatic neuroendocrine cancer, an islet cell cancer, or a pancreatic endocrine cancer. Further provided herein are compositions, wherein the subject has a metastatic cancer. Further provided herein are compositions, wherein the at least one nucleic acid encodes for two or more cancer-associated proteins, wherein the two or more cancer-associated proteins are selected from:(i) epidermal growth factor receptor (EGFR); (ii) vascular endothelial growth factor (VEGF); (iii) Wilms tumor 1 (WTT); (iv) preferentially expressed antigen of melanoma (PRAME); (v) PR1; (vi) proteinase 3; (vii) elastase; (viii) cathepsin G; (ix) survivin; (x) New York esophagus 1 (NY-Eso-l); (xi) melanoma-associated antigen (MAGE); (xii) tyrosinase; (xiii) glycoprotein 100 (gp100); (xiv) carcinoembryonic antigen (CEA); (xv) mucin; (xvi) fibroblast growth factor (FGF); (xvii) programmed cell death protein (PD-1); (xviii) metastasis tumor antigen (MTA); (xix) human epidermal growth factor receptor 2 (Her2); (xx) Mammaglobin A (SCGB2A2); (xxi) alpha lactalbumin (LALBA); (xxii) cyclin D1 (CCND1); (xxiii) folate receptor 1 (FOLR1); (xxiv) telomerase (TERT); (xxv) RecQ protein-like (DNA helicase Q1-like) (RECQL); (xxvi) leptin receptor (LEPR); (xxvii) ERBB receptor feedback inhibitor 1(ERRFI1); (xxviii) lysosomal protein transmembrane 4 alpha (LAPTM4A); (xxix) Kirsten rat sarcoma virus (K-Ras); (xxx) S100 protein; (xxxi) a cluster of differentiation (CD) family protein; (xxxii) alphafetoprotein (AFP); (xxxiii) epithelial tumor antigen (ETA); (xxxiv) tumor protein p53; (xxxv) ephrin receptor; (xxxvi) transferrin receptor; (xxxvii) neoglycoprotein; (xxxviii) tumor necrosis factor (TNF)-alpha (α) receptor; (xxxvix) human papillomavirus-E6; (xl) human papillomavirus-E7; (xli) cytokeratin; (xlii) beta-catenin; (xliii) carboxypeptidase M; (xliv) EP4 receptor; (xlv) human milk fat glomeruli antigen; (xlvi) tumor necrosis factor (TNF)-beta ($) receptor; (xlvii) B7-1 protein; (xlviii) B7-2 protein; (xlix) TNF receptor-associated factor 2; (1) Melanoma-associated antigen recognized by T cells 1 (MART-1); and functional fragments thereof. Further provided herein are compositions, wherein the MAGE is MAGE-Ai, MAGE-A3, MART-I/Melan-A, MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-All, or MAGE-A12. Further provided herein are compositions, the cluster of differentiation family protein is CD5, CD19, CD20, CD22, CD23, CD25, CD27, CD30, CD33, CD36, CD46, CD52, CD79a, CD79b, CD123, or CD317. wherein the plurality of nucleic acids comprises sequence encoding for any one of. SEQ ID NOS: 3-70, 72-74, 77, 78, 87. Further provided herein are compositions, wherein the sequence comprises one of more of SEQ ID NOS: 1, 2, 71, 75, 76, 80-86, 88, 89. Further provided herein are compositions, the RNA polymerase complex region is downstream of a subgenomic promoter from an alphavirus. Further provided herein are compositions, wherein RNA polymerase complex region encodes for an RNA-dependent RNA polymerase. Further provided herein are compositions, wherein the RNA-dependent RNA polymerase is Venezuelan equine encephalitis virus (VEEV) RNA polymerase. Further provided herein are compositions, wherein the RNA polymerase complex region comprises a sequence of SEQ ID NO: 71. Further provided herein are compositions, wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis(azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; $3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′,9″″,19″′″-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis(octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein each lipid carrier further comprises a hydrophobic core. Further provided herein are compositions, wherein the hydrophobic core comprises an oil. Further provided herein are compositions, wherein the oil is in liquid phase. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein each lipid carrier comprises a cationic lipid and an oil. Further provided herein are compositions, wherein the hydrophilic surfactant is polysorbate. Further provided herein are compositions, wherein the hydrophobic surfactant is a phosphorous-terminated surfactant, a carboxylate-terminated surfactant, a sulfate-terminated surfactant, an amine-terminated surfactant, trioctylphosphine oxide (TOPO), or distearyl phosphatidic acid (DSPA). Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the compositions comprise: a plurality of nucleic acids, wherein the plurality of nucleic acids comprises sequence encoding separately for: an antibody or a functional fragment thereof; and an RNA polymerase complex region; and a plurality of nanoparticles, wherein each nanoparticle comprises a cationic surface, and wherein the at least one nucleic acid is complexed to the cationic surface.

Provided herein are compositions, wherein the compositions comprise: a plurality of nucleic acids, wherein the plurality of nucleic acids comprises sequence encoding separately for: an antibody or a functional fragment thereof; and an RNA polymerase complex region; and a plurality of nanoparticles, wherein each nanoparticle comprises a cationic surface, and wherein the at least one nucleic acid is complexed to the cationic surface. Further provided herein are compositions, wherein the an antibody or a functional fragment thereof is a cancer therapeutic antibody. Further provided herein are compositions, wherein the antibody is a monoclonal antibody. Further provided herein are compositions, wherein the antibody, the antibody fragment, or the functional fragment thereof is a murine antibody, a camelid antibody, a humanized antibody, or a fully human antibody. Further provided herein are compositions, wherein the antibody is an immunoglobulin (Ig) molecule or a functional fragment thereof. Further provided herein are compositions, wherein the immunoglobulin molecule is an IgG, IgE, IgM, IgD, IgA, or an IgY isotype immunoglobulin molecule or a functional fragment thereof. Further provided herein are compositions, wherein the immunoglobulin molecule of the functional fragment comprises at least a fragment of an IgG1, an IgG2, an IgG3, an IgG4, an IgGA1, or an IgGA2 subclass immunoglobulin molecule. Further provided herein are compositions, wherein the antibody or the functional fragment thereof is recombinant, chimeric, or multivalent. Further provided herein are compositions, wherein the antibody or the functional fragment thereof is a bispecific antibody, a trispecific antibody, a multispecific antibody, or a functional fragment thereof. Further provided herein are compositions, wherein the antibody or the functional fragment is an antigen-binding fragment (Fab), and Fab2a F(ab′), a F(ab′)2, an dAb, an Fc, a Fv, a disulfide linked Fv, a scFv, a tandem scFv, a free LC, a half antibody, a single domain antibody (dAb), a diabody, or a nanobody. Further provided herein are compositions, wherein the antibody or functional fragment thereof specifically binds to a cancer-associated protein or a microbial antigen. Further provided herein are compositions, wherein the antibody or functional fragment thereof is atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab. Further provided herein are compositions, wherein the at least one nucleic acid is RNA or DNA. Further provided herein are compositions, wherein the plurality of cancer-associated proteins are expressed by one or more cancer cells of a subject. Further provided herein are compositions, wherein the subject has a solid tumor or a blood cancer. Further provided herein are compositions, wherein the solid tumor is a carcinoma, a melanoma, or a sarcoma. Further provided herein are compositions, wherein the blood cancer is lymphoma or leukemia. Further provided herein are compositions, wherein the subject has a skin cancer. Further provided herein are compositions, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are compositions, wherein the subject has a lung cancer. Further provided herein are compositions, wherein the lung cancer is a non-small cell lung cancer (NSCLC) or a small cell lung cancer (SCLC). Further provided herein are compositions, wherein the NSCLC is an adenocarcinoma, a squamous cell carcinoma, a large cell carcinoma, an adenosquamous carcinoma, or a sarcomatoid carcinoma. Further provided herein are compositions, the subject has a pancreatic cancer. Further provided herein are compositions, wherein the pancreatic cancer is a pancreatic adenocarcinoma, a pancreatic exocrine cancer, a pancreatic neuroendocrine cancer, an islet cell cancer, or a pancreatic endocrine cancer. Further provided herein are compositions, wherein the subject has a metastatic cancer. Further provided herein are compositions, wherein the at least one nucleic acid encodes for two or more cancer-associated proteins, wherein the two or more cancer-associated proteins are selected from:(i) epidermal growth factor receptor (EGFR); (ii) vascular endothelial growth factor (VEGF); (iii) Wilms tumor 1 (WT1); (iv) preferentially expressed antigen of melanoma (PRAME); (v) PRI; (vi) proteinase 3; (vii) elastase; (viii) cathepsin G; (ix) survivin; (x) New York esophagus 1 (NY-Eso-l); (xi) melanoma-associated antigen (MAGE); (xii) tyrosinase; (xiii) glycoprotein 100 (gp100); (xiv) carcinoembryonic antigen (CEA); (xv) mucin; (xvi) fibroblast growth factor (FGF); (xvii) programmed cell death protein (PD-1); (xviii) metastasis tumor antigen (MTA); (xix) human epidermal growth factor receptor 2 (Her2); (xx) Mammaglobin A (SCGB2A2); (xxi) alpha lactalbumin (LALBA); (xxii) cyclin D1 (CCND1); (xxiii) folate receptor 1 (FOLR1); (xxiv) telomerase (TERT); (xxv) RecQ protein-like (DNA helicase Q1-like) (RECQL); (xxvi) leptin receptor (LEPR); (xxvii) ERBB receptor feedback inhibitor 1(ERRFI1); (xxviii) lysosomal protein transmembrane 4 alpha (LAPTM4A); (xxix) Kirsten rat sarcoma virus (K-Ras); (xxx) S100 protein; (xxxi) a cluster of differentiation (CD) family protein; (xxxii) alphafetoprotein (AFP); (xxxiii) epithelial tumor antigen (ETA); (xxxiv) tumor protein p53; (xxxv) ephrin receptor; (xxxvi) transferrin receptor; (xxxvii) neoglycoprotein; (xxxviii) tumor necrosis factor (TNF)-alpha (α) receptor; (xxxvix) human papillomavirus-E6; (xl) human papillomavirus-E7; (xli) cytokeratin; (xlii) beta-catenin; (xliii) carboxypeptidase M; (xliv) EP4 receptor; (xlv) human milk fat glomeruli antigen; (xlvi) tumor necrosis factor (TNF)-beta ($) receptor; (xlvii) B7-1 protein; (xlviii) B7-2 protein; (xlix) TNF receptor-associated factor 2; (1) Melanoma-associated antigen recognized by T cells 1 (MART-1); and functional fragments thereof. Further provided herein are compositions, wherein the MAGE is MAGE-A1, MAGE-A3, MART-I/Melan-A, MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-All, or MAGE-A12. Further provided herein are compositions, the cluster of differentiation family protein is CD5, CD19, CD20, CD22, CD23, CD25, CD27, CD30, CD33, CD36, CD46, CD52, CD79a, CD79b, CD123, or CD317. wherein the plurality of nucleic acids comprises sequence encoding for any one of. SEQ ID NOS: 3-70, 72-74, 77, 78, 87. Further provided herein are compositions, wherein the sequence comprises one of more of SEQ ID NOS: 1, 2, 71, 75, 76, 80-86, 88, 89. Further provided herein are compositions, the RNA polymerase complex region is downstream of a subgenomic promoter from an alphavirus. Further provided herein are compositions, wherein RNA polymerase complex region encodes for an RNA-dependent RNA polymerase. Further provided herein are compositions, wherein the RNA-dependent RNA polymerase is Venezuelan equine encephalitis virus (VEEV) RNA polymerase. Further provided herein are compositions, wherein the RNA polymerase complex region comprises a sequence of SEQ ID NO: 71. Further provided herein are compositions, wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis(azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; $3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″,9″″′-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein each lipid carrier further comprises a hydrophobic core. Further provided herein are compositions, wherein the hydrophobic core comprises an oil. Further provided herein are compositions, wherein the oil is in liquid phase. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein each lipid carrier comprises a cationic lipid and an oil. Further provided herein are compositions, wherein the hydrophilic surfactant is polysorbate. Further provided herein are compositions, wherein the hydrophobic surfactant is a phosphorous-terminated surfactant, a carboxylate-terminated surfactant, a sulfate-terminated surfactant, an amine-terminated surfactant, trioctylphosphine oxide (TOPO), or distearyl phosphatidic acid (DSPA). Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the compositions comprise: a plurality of nucleic acids, wherein the plurality of nucleic acids comprises sequence encoding separately for: an antibody or a functional fragment thereof; and an RNA polymerase complex region; and a plurality of nanoparticles, wherein each nanoparticle comprises a cationic surface, and wherein the at least one nucleic acid is complexed to the cationic surface.

Provided herein are pharmaceutical compositions comprising any one of the compositions provided herein; and a pharmaceutically acceptable excipient.

Provided herein are methods for modulating an immune response, wherein the methods comprise: administering to a subject having cancer the composition provided herein or a pharmaceutical composition provided herein.

Provided herein are methods for modulating an immune response, wherein the methods comprise: administering to a subject having a cancer, a composition, wherein the composition comprises: at least one nucleic acid, wherein the at least one nucleic acid comprises a sequence encoding for a plurality of cancer-associated proteins, wherein, prior to the administering, the plurality of cancer-associated proteins are increased in presence compared to non-cancer cells of the subject or comprise a sequence modification compared to non-cancer cells of the subject; and a plurality of nanoparticles, wherein each nanoparticle comprises a cationic surface, and wherein the at least one nucleic acid is complexed to the cationic surface. Further provided herein are methods, wherein the methods further comprise screening cancer cells of the subject for proteins or nucleic acids with increased presence or sequence modification compared to non-cancer cells. Further provided herein are methods, the subject has a solid tumor or a blood cancer. Further provided herein are methods, wherein the solid tumor is a carcinoma, a melanoma, or a sarcoma. Further provided herein are methods, wherein the blood cancer is lymphoma or leukemia. Further provided herein are methods, wherein the subject has a metastatic cancer. Further provided herein are methods, wherein at least one cancer-associated protein of the plurality of cancer-associated proteins is a protein expressed by a melanoma cell. Further provided herein are methods, wherein the plurality of cancer-associated proteins are selected from: (i) epidermal growth factor receptor (EGFR); (ii) vascular endothelial growth factor (VEGF); (iii) Wilms tumor 1 (WTT); (iv) preferentially expressed antigen of melanoma (PRAME); (v) PR1; (vi) proteinase 3; (vii) elastase; (viii) cathepsin G; (ix) survivin; (x) New York esophagus 1 (NY-Eso-l); (xi) melanoma-associated antigen (MAGE); (xii) tyrosinase; (xiii) glycoprotein 100 (gp100); (xiv) carcinoembryonic antigen (CEA); (xv) mucin; (xvi) fibroblast growth factor (FGF); (xvii) programmed cell death protein (PD-1); (xviii) metastasis tumor antigen (MTA); (xix) human epidermal growth factor receptor 2 (Her2); (xx) Mammaglobin A (SCGB2A2); (xxi) alpha lactalbumin (LALBA); (xxii) cyclin D1 (CCND1); (xxiii) folate receptor 1 (FOLR1); (xxiv) telomerase (TERT); (xxv) RecQ protein-like (DNA helicase Q1-like) (RECQL); (xxvi) leptin receptor (LEPR); (xxvii) ERBB receptor feedback inhibitor 1(ERRFI1); (xxviii) lysosomal protein transmembrane 4 alpha (LAPTM4A); (xxix) Kirsten rat sarcoma virus (K-Ras); (xxx) S100 protein; (xxxi) a cluster of differentiation (CD) family protein; (xxxii) alphafetoprotein (AFP); (xxxiii) epithelial tumor antigen (ETA); (xxxiv) tumor protein p53; (xxxv) ephrin receptor; (xxxvi) transferrin receptor; (xxxvii) neoglycoprotein; (xxxviii) tumor necrosis factor (TNF)-alpha (a) receptor; (xxxvix) human papillomavirus-E6; (xl) human papillomavirus-E7; (xli) cytokeratin; (xlii) beta-catenin; (xliii) carboxypeptidase M; (xliv) EP4 receptor; (xlv) human milk fat glomeruli antigen; (xlvi) tumor necrosis factor (TNF)-beta ($) receptor; (xlvii) B7-1 protein; (xlviii) B7-2 protein; (xlix) TNF receptor-associated factor 2; (1) Melanoma-associated antigen recognized by T cells 1 (MART-1); and functional fragments thereof. Further provided herein are methods, wherein the MAGE is MAGE-A1, MAGE-A3, MART-I/Melan-A, MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-All, or MAGE-A12. Further provided herein are methods, wherein the cluster of differentiation family protein is CD5, CD19, CD20, CD22, CD23, CD25, CD27, CD30, CD33, CD36, CD46, CD52, CD79a, CD79b, CD123, or CD317. Further provided herein are methods, wherein the plurality of nucleic acids comprises sequence encoding for any one of SEQ ID NOS: 3-7, 72, 74, 77, 78, 87. Further provided herein are methods, wherein the sequences comprises one of more of SEQ ID NOS: 1, 2, 71, 75, 76, 80-86, 88, 89. Further provided herein are methods, wherein the at least one nucleic acid further comprises an RNA polymerase complex region. Further provided herein are methods, wherein the RNA polymerase complex region is downstream of a subgenomic promoter from an alphavirus. Further provided herein are methods, wherein the RNA polymerase complex region encodes for an RNA-dependent RNA polymerase. Further provided herein are methods, wherein the RNA-dependent RNA polymerase is Venezuelan equine encephalitis virus (VEEV) RNA polymerase. Further provided herein are methods, wherein the cationic surface comprises a cationic lipid. Further provided herein are methods, wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl)bis(propane-3,1 diyl))bis(azanetriyl))tetrapropionate, 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; $3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9″″′-((((benzene-1,3,5-tricarbonyl)yris(azanediyl))tris(propane-3,1-diyl))tris(azanetriyl))hexanonanoate; Lipid H(SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis(octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are methods, wherein each nanoparticle further comprises a hydrophobic core. Further provided herein are methods, wherein the hydrophobic core comprises an oil. Further provided herein are methods, wherein the oil is in liquid phase. Further provided herein are methods, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are methods, wherein the triglyceride is capric triglyceride, caprylic triglyceride, a caprylic and capric triglyceride, a triglyceride ester, or myristic acid triglycerin. Further provided herein are methods, wherein each nanoparticle comprises a cationic lipid and an oil. Further provided herein are methods, wherein each nanoparticle further comprises a surfactant. Further provided herein are methods, wherein the surfactant is polysorbate, a phosphorous-terminated surfactant, a carboxylate-terminated surfactant, a sulfate-terminated surfactant, an amine-terminated surfactant, trioctylphosphine oxide (TOPO), or distearyl phosphatidic acid (DSPA). Further provided herein are methods, wherein the administering is local administration or systemic administration. Further provided herein are methods, wherein the administering is via an intratumoral, subcutaneous, intradermal, intramuscular, inhalation, intravenous, intraperitoneal, intracranial, or intrathecal route. Further provided herein are methods, wherein the composition is administered with a cancer therapeutic antibody or the composition provided herein. Further provided herein are methods, wherein the administering results in a reduction of a tumor size or a reduction in a tumor volume in the subject. Further provided herein are methods, wherein the administering results in a reduction of a cancer recurrence. Further provided herein are methods, wherein the administering results in a reduction in tumor metastasis.

Provided herein is a method of prophylactically immunizing a subject for a cancer, the method comprising: administering to a subject the composition provided herein or the pharmaceutical composition provided herein, thereby immunizing the subject to a cancer. Further provided herein are methods, wherein the administering is via an intratumoral, subcutaneous, intradermal, intramuscular, inhalation, intravenous, intraperitoneal, intracranial, or intrathecal route. Further provided herein are methods, wherein the subject is at risk for developing a skin cancer. Further provided herein are methods, wherein the skin cancer is a basal cell cancer, a melanoma, a Merkel cell cancer, a squamous cell carcinoma, a cutaneous lymphoma, a Kaposi sarcoma, or a skin adnexal cancer.

Provided herein are methods for the treatment of a cancer in a subject, the methods comprising: administering to the subject a composition provided herein or a pharmaceutical composition provided herein, thereby treating the cancer in the subject. Further provided herein are methods, wherein the administering is via an intratumoral, subcutaneous, intradermal, intramuscular, inhalation, intravenous, intraperitoneal, intracranial, or intrathecal route. Further provided herein are methods, wherein the composition is administered with a cancer therapeutic antibody or the composition provided herein. Further provided herein are methods, wherein the subject has a solid tumor or a blood cancer. Further provided herein are methods, wherein the solid tumor is a carcinoma, a melanoma, or a sarcoma. Further provided herein are methods, wherein the blood cancer is lymphoma or leukemia. Further provided herein are methods, wherein the subject has a skin cancer. Further provided herein are compositions, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are methods, wherein the subject has a lung cancer. Further provided herein are methods, wherein the lung cancer is a non-small cell lung cancer (NSCLC) or a small cell lung cancer (SCLC). Further provided herein are compositions, wherein the NSCLC is an adenocarcinoma, a squamous cell carcinoma, a large cell carcinoma, an adenosquamous carcinoma, or a sarcomatoid carcinoma. Further provided herein are methods, the subject has a pancreatic cancer. Further provided herein are methods, wherein the pancreatic cancer is a pancreatic adenocarcinoma, a pancreatic exocrine cancer, a pancreatic neuroendocrine cancer, an islet cell cancer, or a pancreatic endocrine cancer. Further provided herein are methods, wherein the subject has a metastatic cancer. Further provided herein are methods, wherein the administering results in a reduction of a tumor size or a reduction in a tumor volume in the subject. Further provided herein are methods, wherein the administering results in a reduction of a cancer recurrence. Further provided herein are methods, wherein the administering results in a reduction in tumor metastasis.

Provided herein are methods for the personalized treatment of cancer in a subject, the methods comprising: (a) receiving the results of an assay that indicates that the subject has a tumor, wherein the tumor comprises a cancer-associated protein; (b) administering to the subject a composition provided herein, wherein the composition comprises at least one nucleic acid encoding for the cancer-associated protein in (a), thereby treating the cancer in the subject. Further provided herein are methods, wherein the administering is via an intratumoral, subcutaneous, intradermal, intramuscular, inhalation, intravenous, intraperitoneal, intracranial, or intrathecal route. Further provided herein are methods, wherein the composition is administered with a cancer therapeutic antibody or the composition provided herein. Further provided herein are methods, wherein the subject has a solid tumor or a blood cancer. Further provided herein are methods, wherein the solid tumor is a carcinoma, a melanoma, or a sarcoma. Further provided herein are methods, wherein the blood cancer is lymphoma or leukemia. Further provided herein are methods, wherein the subject has a skin cancer. Further provided herein are compositions, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are methods, wherein the subject has a lung cancer. Further provided herein are methods, wherein the lung cancer is a non-small cell lung cancer (NSCLC) or a small cell lung cancer (SCLC). Further provided herein are compositions, wherein the NSCLC is an adenocarcinoma, a squamous cell carcinoma, a large cell carcinoma, an adenosquamous carcinoma, or a sarcomatoid carcinoma. Further provided herein are methods, the subject has a pancreatic cancer. Further provided herein are methods, wherein the pancreatic cancer is a pancreatic adenocarcinoma, a pancreatic exocrine cancer, a pancreatic neuroendocrine cancer, an islet cell cancer, or a pancreatic endocrine cancer. Further provided herein are methods, wherein the subject has a metastatic cancer. Further provided herein are methods, wherein the administering results in a reduction of a tumor size or a reduction in a tumor volume in the subject. Further provided herein are methods, wherein the administering results in a reduction of a cancer recurrence. Further provided herein are methods, wherein the administering results in a reduction in tumor metastasis.

Provided herein are methods for the personalized treatment of cancer in a subject, the methods comprising: (a) receiving the results of an assay that indicates that the subject has a tumor, wherein the tumor comprises a cancer-associated protein; (b) administering to the subject a composition provided herein, wherein the composition comprises at least one nucleic acid encoding for an antibody that specifically binds to the cancer-associated protein in (a), thereby treating the cancer in the subject. Further provided herein are methods, wherein the administering is via an intratumoral, subcutaneous, intradermal, intramuscular, inhalation, intravenous, intraperitoneal, intracranial, or intrathecal route. Further provided herein are methods, wherein the composition is administered with at least one additional cancer therapeutic agent. Further provided herein are methods, wherein the subject has a solid tumor or a blood cancer. Further provided herein are methods, wherein the solid tumor is a carcinoma, a melanoma, or a sarcoma. Further provided herein are methods, wherein the blood cancer is lymphoma or leukemia. Further provided herein are methods, wherein the subject has a skin cancer. Further provided herein are compositions, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are methods, wherein the subject has a lung cancer. Further provided herein are methods, wherein the lung cancer is a non-small cell lung cancer (NSCLC) or a small cell lung cancer (SCLC). Further provided herein are compositions, wherein the NSCLC is an adenocarcinoma, a squamous cell carcinoma, a large cell carcinoma, an adenosquamous carcinoma, or a sarcomatoid carcinoma. Further provided herein are methods, the subject has a pancreatic cancer. Further provided herein are methods, wherein the pancreatic cancer is a pancreatic adenocarcinoma, a pancreatic exocrine cancer, a pancreatic neuroendocrine cancer, an islet cell cancer, or a pancreatic endocrine cancer. Further provided herein are methods, wherein the subject has a metastatic cancer. Further provided herein are methods, wherein the administering results in a reduction of a tumor size or a reduction in a tumor volume in the subject. Further provided herein are methods, wherein the administering results in a reduction of a cancer recurrence. Further provided herein are methods, wherein the administering results in a reduction in tumor metastasis.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: Techniques and Materials for the Production of Lipid Nanoparticles

The following materials were used in the manufacturing of lipid-inorganic nanoparticles (i.e., lipid nanoparticles). The compositions, kits and methods described herein are not limited to the techniques or materials described herein.

Iron oxide nanoparticles at 25 mg Fe/ml in chloroform and of various average diameters (5, 10, 15, 20, 25 and 30 nm) were purchased from Ocean Nanotech (San Diego, CA, USA). Squalene and SPAN® 60 (sorbitan monostearate) were purchased from Millipore Sigma. TWEEN® 80 (polyethylene glycol sorbitan monooleate) and sodium citrate dihydrate were purchased from Fisher Chemical. The chloride salt of the cationic lipid 1,2-dioleoyl-3-trimethylammonium-propane (DOTAP chloride) was purchased from Corden Pharma. Ultrapure water (18.2 mega ohm-centimeter (MOhm-cm) resistivity) was obtained from a Milli-Q water purification system (Millipore Sigma).

The lipid carrier comprises squalene, sorbitan monostearate (e.g., SPAN® 60), polysorbate 80 (e.g., TWEEN® 80), DOTAP chloride, iron oxide nanoparticles and sodium citrate dihydrate. In general, to iron oxide nanoparticles with a number-weighted average diameter of 5 nm, chloroform was added. Chloroform was allowed to evaporate in a fume hood leaving behind a dry coating of iron oxide nanoparticles. To the iron oxide nanoparticles, SPAN® 60, squalene, and DOTAP chloride were added to prepare the “oil” phase.

The oil phase was sonicated 30 minutes in a water bath pre-heated to 60° C. Separately, in a 1 liter glass bottle, the “aqueous” phase was prepared by adding TWEEN® 80 to sodium citrate dihydrate solution prepared with Milli-Q water.

The aqueous phase was stirred for 30 minutes to allow complete dissolution of TWEEN® 80. After complete dissolution of TWEEN® 80, the aqueous phase was transferred to a beaker and incubated in a water bath pre-heated to 60° C. To the heated oil phase, the pre-heated aqueous phase was added.

The mixture was immediately emulsified using a VWR® 200 homogenizer (VWR International) until a homogenous colloid with a milk-like appearance was produced. The colloid was subsequently processed by passaging the fluid through a Y-type interaction chamber of a LM10 microfluidizer at 20,000 psi.

The fluid was passaged until the z-average hydrodynamic diameter, measured by dynamic light scattering (Malvern Zetasizer Nano S), was 59 nm with a 0.2 polydispersity index.

The microfluidized lipid carrier sample was terminally filtered with a 200 nm pore-size polyethersulfone (PES) syringe filter.

### Example 2: Exemplary Techniques and Materials for Producing Lipid Nanoparticles

The following materials were used in the manufacturing of lipid-inorganic nanoparticles (i.e., lipid nanoparticles). The compositions, kits and methods described herein are not limited to the techniques or materials describe herein.

Iron oxide nanoparticles at 25 mg Fe/ml in chloroform and of various average diameters (5, 10, 15, 20, 25 and 30 nm) were purchased from Ocean Nanotech (San Diego, CA). Squalene and SPAN® 60 (sorbitan monostearate) were purchased from Millipore Sigma. TWEEN® 80 (polyethylene glycol sorbitan monooleate) and sodium citrate dihydrate were purchased from Fisher Chemical. The chloride salt of the cationic lipid 1,2-dioleoyl-3-trimethylammonium-propane (DOTAP chloride) was purchased from Corden Pharma. Ultrapure water (18.2 MOhm-cm resistivity) was obtained from a Milli-Q water purification system (Millipore Sigma).

Lipid carriers were prepared which comprised 37.5 mg/ml squalene, 37 mg/ml SPAN® 60, 37 mg/ml TWEEN® 80, 30 mg/ml DOTAP chloride, 0.1 mg/ml 10 nm iron oxide nanoparticles and 10 mM sodium citrate dihydrate.

The lipid carriers were manufactured using the following procedures. In a 200 ml beaker, 0.4 ml of iron oxide nanoparticles at 25 mg Fe/ml in chloroform, with a number-weighted average diameter of 10 nm, were added.

Chloroform was allowed to evaporate in a fume hood leaving behind a dry coating of iron oxide nanoparticles. To the iron oxide nanoparticles, 3.7 grams of SPAN® 60, 3.75 grams of squalene, and 3 grams of DOTAP chloride were added to prepare the “oil” phase.

The oil phase was sonicated 30 minutes in a water bath pre-heated to 60° C. Separately, in a 1 liter glass bottle, the “aqueous” phase was prepared by adding 39 grams of TWEEN® 80 to 1,000 ml 10 mM sodium citrate dihydrate solution prepared with Milli-Q water.

The aqueous phase was stirred for 30 minutes to allow complete dissolution of TWEEN® 80. After complete dissolution of TWEEN® 80, 96 ml of the aqueous phase was transferred to a 200 ml beaker and incubated in a water bath pre-heated to 60° C. To the heated oil phase, 96 ml of the pre-heated aqueous phase was added. The mixture was immediately emulsified using a VWR® 200 homogenizer (VWR International) until a homogenous colloid with a milk-like appearance was produced. The colloid was subsequently processed by passaging the fluid through a Y-type interaction chamber of a LM10 microfluidizer at 20,000 psi. The fluid was passaged until the z-average hydrodynamic diameter, measured by dynamic light scattering (Malvern Zetasizer Nano S), was 54 nm with a 0.2 polydispersity index. The microfluidized lipid carrier sample was terminally filtered with a 200 nm pore-size polyethersulfone (PES) syringe filter.

### Example 3: Exemplary Techniques and Materials for Producing Nanoparticles Described Herein

Lipid carriers were prepared which comprised 37.5 mg/ml squalene, 37 mg/ml SPAN® 60, 37 mg/ml TWEEN® 80, 30 mg/ml DOTAP chloride, 0.2 mg/ml 15 nm iron oxide nanoparticles, and 10 M sodium citrate dihydrate. The lipid carriers of Example 9 were manufactured using the following procedures.

In a 200 ml beaker, 0.8 ml of iron oxide nanoparticles at 25 mg Fe/ml in chloroform, with a number-weighted average diameter of 15 nm, was added. Chloroform was allowed to evaporate in a fume hood leaving behind a dry coating of iron oxide nanoparticles. To the iron oxide nanoparticles, 3.7 grams of SPAN® 60, 3.75 grams of squalene, and 3 grams of DOTAP chloride were added to prepare the “oil” phase.

The oil phase was sonicated 30 minutes in a water bath pre-heated to 60° C. Separately, in a 1 liter glass bottle, the “aqueous” phase was prepared by adding 39 grams of TWEEN® 80 to 1,000 ml of 10 mM sodium citrate dihydrate solution prepared with Milli-Q water. The aqueous phase was stirred for 30 minutes to allow complete dissolution of TWEEN® 80.

After complete dissolution of TWEEN® 80, 96 ml of the aqueous phase was transferred to a 200 ml beaker and incubated in a water bath pre-heated to 60° C. To the heated oil phase, 96 ml of the pre-heated aqueous phase was added. The mixture was immediately emulsified using a VWR® 200 homogenizer (VWR International) until a homogenous colloid with a milk-like appearance was produced. The colloid was subsequently processed by passaging the fluid through a Y-type interaction chamber of a LM10 microfluidizer at 20,000 psi.

The fluid was passaged until the z-average hydrodynamic diameter, measured by dynamic light scattering (Malvern Zetasizer Nano S), was 52 nm with a 0.2 polydispersity index. The microfluidized lipid carrier sample was terminally filtered with a 200 nm pore-size polyethersulfone (PES) syringe filter.

### Example 4: Composition for Use as a Cancer Vaccine Prepared Using the Construct of SEQ ID NO: 75 or SEQ ID NO: 76

Lipid carrier-RNA complexes are prepared and aliquoted for lyophilization. Samples are lyophilized and then collected and selected for reconstitution. All lyophilized cakes are then reconstituted in 0.7 ml milliQ® water. Table 4 discloses exemplary materials used in the preparation of lipid carrier-RNA complexes.

TABLE 4
Conditions.

| Name | Molecular weight | Concentration |
|---|---|---|
| lipid carrier | | 30 mg DOTAP/ml |
| repRNA comprising sequence | | 1015 ng RNA/μl |
| set forth in SEQ ID NO: 1, SEQ | | |
| ID NO: 2, SEQ ID NO: 75, or | | |
| SEQ ID NO: 76 | | |
| Sucrose | 342.3 | |
| D-Glucose | 180.16 | |
| D-Mannitol | 182.17 | |
| Maltose monohydrate | 360.31 | |
| Trehalose dihydrate | 378.33 | |
| Sodium citrate | | 1M |

Exemplary conditions for lyophilization are set forth as below in Tables 5-7.

TABLE 5
Lyophilization Cycle # 1.

| Time [hr] | T [C.] | P [mTorr] |
|---|---|---|
| 0 | 20 | 760000 |
| 1.5 | −50 | 760000 |
| 2 | −50 | 760000 |
| 2.1 | −50 | 50 |
| 2.5 | −30 | 50 |
| 20 | −30 | 50 |
| 20.5 | 25 | 50 |
| 22 | 25 | 50 |
| 22.1 | 25 | 760000 |

TABLE 6
Lyophilization Cycle # 2.

| Time [hr] | T [C.] | P [mTorr] |
|---|---|---|
| 0 | 20 | 760000 |
| 1.5 | −65 | 760000 |
| 2 | −65 | 760000 |
| 2.1 | −65 | 15 |
| 2.5 | −50 | 15 |
| 24 | −50 | 15 |
| 24.5 | 25 | 15 |
| 26 | 25 | 15 |
| 26.1 | 25 | 760000 |

TABLE 7
Lyophilization Cycle # 3.

| Time [hr] | T [C.] | P [mTorr] |
|---|---|---|
| 0 | 20 | 760000 |
| 1.5 | −65 | 760000 |
| 2 | −65 | 760000 |
| 2.1 | −65 | 15 |
| 2.5 | −50 | 15 |
| 26.5 | −50 | 15 |
| 26.6 | −30 | 15 |
| 46 | −30 | 15 |
| 48 | 25 | 15 |
| 48.1 | 25 | 760000 |

Preparation of diluents: The diluents comprising the sugar and citrate were prepared as outlined in Table 8. Each sugar was weighed out in a 50 ml RNase free conical tube. About 35-40 ml of nuclease free water was added to dissolve the sugar and slight heat and sonication was used, as needed. Pipette-in 0.5 ml of 1M Na-citrate, pH=6 solution. After all sugar has dissolved and solution is clear, Q.S. with nuclease free water to 50 ml mark in conical tube. Filter diluents with 0.22 μm STERIFLIP® and screw on cap aseptically to maintain sterility.

TABLE 8
Preparation of diluents

| | | Total | | Calcu- |
|---|---|---|---|---|
| | | Vol- | Actual | lated |
| | Composition | ume | mass | conc |
| Diluent | ID | [ml] | [g] | [g/L] |
| 22% sucrose/10 mM citrate | 22% sucrose | 50.0 | 11.1 | 222.0 |
| 50% sucrose/10 mM citrate | 50% sucrose | 50.0 | 25.0 | 500.0 |
| 22% maltose/10 mM citrate | 22% maltose | 50.0 | 11.7 | 233.7 |
| 22% trehalose/10 mM citrate | 22% trehalose | 50.0 | 12.3 | 245.4 |
| 11% glucose/10 mM citrate | 11% glucose | 50.0 | 5.6 | 111.0 |
| 11% mannitol/10 mM citrate | 11% mannitol | 50.0 | 5.6 | 111.0 |

001991 Preparation of pre-complex formulation: Lipid carrier “DS” is the bulk solution at 30 mg DOTAP/ml and refers to Fe-lipid carrier formulation, whose preparation is described in Example 2.

Lipid carrier “DS” (30 mg DOTAP/ml) 10-fold was diluted in each diluent to make 3 mg DOTAP/ml lipid carrier “DP”, except in 50% sucrose composition lipid carrier 5-fold was diluted to make 2×6 mg DOTAP/ml lipid carrier “DP”. The target RNA concentration in liquid formulation was 50 ng/i, complexed with lipid carrier at N:P of 15. This simulates 25 g RNA dose per vial. Table 9 discloses the preparation of pre-complex lipid carrier complex. The unused lipid carrier was stored at 2-8° C..

TABLE 9
Pre-complex lipid carrier preparation.

| | Lipid Carrier | Diluent | Total |
|---|---|---|---|
| Composition ID | DS [μl] | [μl] | [μl] |
| 22% sucrose | 420 | 3780 | 4200 |
| 50% sucrose | 420 | 1680 | 2100 |
| 22% maltose | 420 | 3780 | 4200 |
| 22% trehalose | 420 | 3780 | 4200 |
| 11% glucose | 420 | 3780 | 4200 |
| 11% mannitol | 420 | 3780 | 4200 |
| Maltose monohydrate | Sigma | 360.31 | |
| Trehalose dihydrate | sigma | 378.33 | |
| Sodium citrate | Teknova | | 1M |

Table 10 discloses the preparation of pre-complex nanostructured lipid carrier (NLC) complex. The NLCs were used as control. The unused NLC was stored at 2-8 degrees Celsius for fresh complex controls.

TABLE 10
Pre-complex NLC preparation.

| Composition ID | NLC [μl] | Diluent [μl] | Total [μl] |
|---|---|---|---|
| 22% sucrose | 420 | 3780 | 4200 |

The preparation of RNA pre-complex is disclosed in Table 11. The RNA stock was prepared. About 7.5 ml or 0.63 ml for 50% sucrose per aliquot were split and stored at −80 degrees Celsius.

TABLE 11
Preparation of RNA pre-complex.

| | | | | Actual RNA |
|---|---|---|---|---|
| | RNA | 5 mM | | concentration |
| | Stock | citrate | Total | pre-complex |
| Composition ID | [μl] | [μl] | [μl] | [ng/μl] |
| all | 2141.4 | 19593.6 | 21735.0 | 107.3 |
| 50% sucrose | 356.9 | 1454.4 | 1811.3 | 221.0 |

The preparation of lipid carrier-RNA complex is disclosed in Table 11. The RNA stock was prepared. The volume of diluted RNA was (+5%) and diluted lipid carrier was (+5%) per complexing per lyophilization (1 yo) cycle.

Complexes of lipid carrier+RNA or NLC+RNA were prepared by mixing 1:1 by volume each diluted formulation listed in the above described Table 10 and Table 11, with the corresponding “Composition ID” diluted RNA disclosed in Table 12. The complexes were equilibrated for 30 minutes at room temperature before subjecting to the lyophilization cycles or long-term storage conditions.

TABLE 12
Preparation of lipid carrier-RNA pre-complex.

| | | | | | | Final |
|---|---|---|---|---|---|---|
| | | Diluted | Diluted | | Final RNA | Sugar |
| | Diluted | lipid | NLC | Total | conc. | conc. |
| Composition ID | RNA | carrier | [μl] | [μl] | [ng/μl] | [% w/v] |
| 22% sucrose | 1102.5 | 1102.5 | | 2205 | 50 | 10 |
| 50% sucrose | 551.25 | 551.25 | | 1102.5 | 100 | 20 |
| 22% maltose | 1102.5 | 1102.5 | | 2205 | 50 | 10 |
| 22% trehalose | 1102.5 | 1102.5 | | 2205 | 50 | 10 |
| 11% glucose | 1102.5 | 1102.5 | | 2205 | 50 | 5 |
| 11% mannitol | 1102.5 | 1102.5 | | 2205 | 50 | 5 |
| 22% sucrose | 1102.5 | | 1102.5 | 2205 | 50 | 10 |

The RNA is a construct having a nucleic acid sequence as set forth in either SEQ ID NO: 75 or SEQ ID NO: 76, each comprising a VEEV RNA sequence backbone and an RNA sequence encoding a cancer-associated protein antigen.

### Example 5: Macrophage Immune Response

Various formulations of lipid carrier and repRNA were prepared and analyzed to assay innate immune response of the lipid carrier in macrophages. Protein expression and stimulation of TNF production in THP-1 macrophages was studied.

Initially, the THP-1 monocytes were differentiated into macrophages using phorbol 12-myristate 13-acetate (PMA). The cells were then transfected with various formulations with Nano Luciferase encoding replicon RNA (SEQ ID NO: 71). The cell culture media was then assessed for NanoLuc and TNF expression.

The formulations and their characteristics such as particle size and PDI that were used in this assay are described in Table 13. The concentration of repRNA encoding NanoLuc was 909 ng/μl and maintained at −80° C.. MIGLYOL® 812 N, a triglyceride ester of saturated coconut/palm-kernel oil derived caprylic and capric fatty acids and plant derived glycerol was used in this assay.

TABLE 13
Formulations.

| | Particle | | | | | | | |
|---|---|---|---|---|---|---|---|---|
| | size | | Iron | | | | | |
| | [diameter, | | [mg | Aluminum | DOTAP | Squalene | MIGLYOL | Solanesol |
| Formulation | nm] | PDI | Fe/ml] | [mg Al/ml] | [mg/ml] | [mg/ml] | [mg/ml] | [mg/ml] |
| Fe-lipid carrier | 59.3 | 0.23 | 0.19 | n/a | 27.9 | 39.4 | n/a | n/a |
| High Fe-lipid | 57.5 | 0.24 | 0.85 | n/a | 29.1 | 40.5 | n/a | n/a |
| carrier | | | | | | | | |
| Fe-lipid carrier | 48.7 | 0.2 | 0.18 | n/a | 28.3 | n/a | not | n/a |
| MIGLYOL | | | | | | | measured | |
| High Fe-lipid | 62.6 | 0.28 | 0.94 | n/a | 27.7 | n/a | not | n/a |
| carrier | | | | | | | measured | |
| MIGLYOL | | | | | | | | |
| Alum-lipid | 64.5 | 0.25 | n/a | 0.88 | 27.4 | 41.2 | n/a | n/a |
| carrier | | | | | | | | |
| Fe-lipid carrier | 86.1 | 0.26 | 0.16 | n/a | 26.2 | n/a | n/a | 36 |
| solanesol (SLN) | | | | | | | | |
| NLC | 50 | 0.26 | n/a | n/a | 26.7 | 34.1 | n/a | n/a |
| CNE | 105.4 | 0.06 | n/a | n/a | 4.4 | 47.4 | n/a | n/a |
| lipid carrier | 54.2 | 0.22 | n/a | n/a | 19.3 | 32.6 | n/a | n/a |
| (w/o IO) | | | | | | | | |

### Example 6: Fe-Lipid Carrier Formulation-NP-i (Prepared at 100 ml Scale)

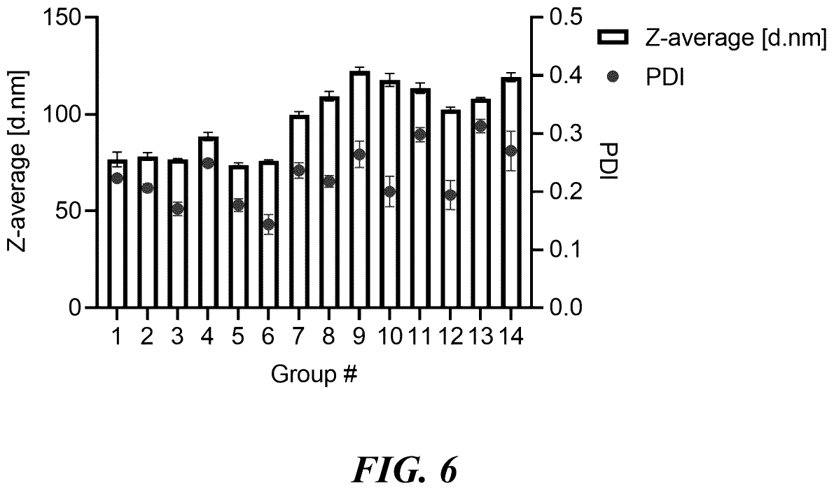

Fe-lipid carrier formulation comprises 37.5 mg/ml squalene (SEPPIC), 37 mg/ml SPAN® 60 (Millipore Sigma), 37 mg/ml TWEEN® 80 (Fisher Chemical), 30 mg/ml DOTAP chloride (LIPOID), 0.2 mg Fe/ml 12 nm oleic acid-coated iron oxide nanoparticles (Imagion Biosystems, San Diego, CA, USA) and 10 mM sodium citrate dihydrate (Fisher Chemical). 1 ml of 20 mg Fe/ml 12 nm diameter oleic acid-coated iron oxide nanoparticles in chloroform (Imagion Biosystems, lot #95-127) were washed three times by magnetically separating in a 4:1 acetone: chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN® 60, and 3 grams DOTAP were added to produce the oil phase. The oil phase was sonicated for 45 minutes in a 65 degree c. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65 degrees Celsius for 30 minutes. The oil phase was mixed with the 92 ml of aqueous phase by adding the warm oil phase to the warm aqueous phase. The mixture was emulsified using a VWR® 200 homogenizer (VWR International, Radnor, PA, USA) and the resulting crude emulsion was processed by passaging through a M110P microfluidizer (Microfluidics, Westwood, MA, USA) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S) -reached 40-80 nm with a 0.1-0.25 polydispersity index (PDI) (FIG. 6). The microfluidized NP-1 formulation was terminally filtered with a 200 nm pore-size polyethersulfone (PES) filter and stored at 2 to 8 degrees C. Iron concentration was determined by inductively coupled plasma-optical emission spectrometry (ICP-OES). DOTAP and squalene concentration were measured by reverse phase high-performance liquid chromatography (RP-HPLC).

### Example 7: High Fe-Lipid Carrier Formulation NP-2 (Prepared at 100 ml Scale)

High Fe-lipid carrier formulation comprises 37.5 mg/ml squalene (SEPPIC), 37 mg/ml SPAN® 60 (Millipore Sigma), 37 mg/ml TWEEN® 80 (Fisher Chemical), 30 mg/ml DOTAP chloride (LIPOID), 1 mg Fe/ml 15 nm oleic acid-coated iron oxide nanoparticles (Imagion Biosystems) and 10 mM sodium citrate dihydrate (Fisher Chemical). 5 ml of 20 mg Fe/ml 15 nm diameter oleic acid-coated iron oxide nanoparticles in chloroform (Imagion Biosystems, Lot #95-133) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN® 60, and 3 grams DOTAP were added to produce the oil phase. The oil phase was sonicated for 45 minutes in a 65 degree Celsius water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S) -reached 40-80 nm with a 0.1-0.3 polydispersity index (PDI). The microfluidized formulation was terminally filtered with a 200 nm pore-size polyethersulfone (PES) filter and stored at 2 to 8 degrees C. Iron concentration was determined by ICP-OES. DOTAP and Squalene concentration were measured by RP-HPLC.

### Example 8: Fe-Lipid Carrier Miglyol Formulation NP-3 (Prepared at 100 ml Scale)

The Fe-lipid carrier miglyol formulation comprises 37.5 mg/ml Miglyol 812 N (IOI Oleo GmbH), 37 mg/ml SPAN® 60 (Millipore Sigma), 37 mg/ml TWEEN® 80 (Fisher Chemical), 30 mg/ml DOTAP chloride (LIPOID), 0.2 mg Fe/ml 15 nm oleic acid-coated iron oxide nanoparticles (Imagion Biosystems) and 10 mM sodium citrate dihydrate (Fisher Chemical). 1 ml of 20 mg Fe/ml 15 nm diameter oleic acid-coated iron oxide nanoparticles in chloroform (Imagion Biosystems, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN® 60, and 3 grams DOTAP were added to produce the oil phase. The oil phase was sonicated for 45 minutes in a 65 degree c. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65 degree c. for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H3OZ-200 μm ceramic interaction chamber until the z-average hydrodynamic diameter—measured by dynamic light scattering (Malvern Zetasizer Nano S) -reached 40-80 nm with a 0.1-0.3 polydispersity index (PDI). The microfluidized formulation was terminally filtered with a 200 nm pore-size polyethersulfone (PES) filter and stored at 2 to 8 degrees C. Iron concentration was determined by ICP-OES. DOTAP concentration was measured by RP-HPLC.

### Example 9: High Fe-Lipid Carrier Miglyol Formulation NP-4 (Prepared at 100 ml Scale)

High Fe-lipid carrier Miglyol formulation comprises 37.5 mg/ml Miglyol 812 N (IOI Oleo GmbH), 37 mg/ml SPAN® 60 (Millipore Sigma), 37 mg/ml TWEEN® 80 (Fisher Chemical), 30 mg/ml DOTAP chloride (LIPOID), 1 mg/ml 15 nm oleic acid-coated iron oxide nanoparticles (ImagionBio) and 10 mM sodium citrate dihydrate (Fisher Chemical). 5 ml of 20 mg Fe/ml 15 nm diameter oleic acid-coated iron oxide nanoparticles in chloroform (ImagionBio, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN® 60, and 3 grams DOTAP were added to produce the oil phase. The oil phase was sonicated for 45 minutes in a 65 degree c. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S) -reached 40-80 nm with a 0.1-0.3 polydispersity index (PDI). The microfluidized formulation was terminally filtered with a 200 nm pore-size polyethersulfone (PES) filter and stored at 2 to 8 degrees C. Iron concentration was determined by ICP-OES. DOTAP concentration was measured by RP-HPLC.

### Example 10: Alum-Lipid Carrier Formulation NP-5 (Prepared at 100 ml Scale)

Alum-lipid carrier formulation comprises 37.5 mg/ml squalene (SEPPIC), 37 mg/ml SPAN® 60 (Millipore Sigma), 37 mg/ml TWEEN® 80 (Fisher Chemical), 30 mg/ml DOTAP chloride (LIPOID), 1 mg A1/ml TOPO-coated Alhydrogel® (aluminum oxyhydroxide) particles (Croda) and 10 mM sodium citrate. 10 ml of Alhydrogel was washed three times in methanol by centrifuging at 1000 rpm for 20 minutes. After the third wash, Alhydrogel was dispersed in 10 ml methanol and to this dispersion was added 1 ml of 250 mg/ml trioctylphosphine oxide (TOPO) and incubated overnight in a 37° C. orbital shaker. Excess TOPO was removed by additional methanol washes and then dispersed in 11 ml methanol. Methanol was allowed to evaporate overnight in the fume hood leaving behind a dry layer of TOPO-Alhydrogel. To this dry TOPO-Alhydrogel layer, 3.75 grams squalene, 3.7 grams SPAN® 60, and 3 grams DOTAP were added to produce the oil phase. The oil phase was sonicated for 45 minutes in a 65 degree c. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S) -reached 40-80 nm with a 0.1-0.3 polydispersity index (PDI). The microfluidized formulation was terminally filtered with a 200 nm pore-size polyethersulfone (PES) filter and stored at 2 to 8 degrees C. Aluminum concentration was determined by ICP-OES. DOTAP and Squalene concentration were measured by RP-HPLC.

### Example 11: Fe-Lipid Carrier Solanesol Formulation NP-6 (Prepared at 100 ml Scale)

Fe-lipid carrier solanesol formulation (NP-6) comprises 37.5 mg/ml Solanesol (Cayman chemicals), 37 mg/ml SPAN® 60 (Millipore Sigma), 37 mg/ml TWEEN® 80 (Fisher Chemical), 30 mg/ml DOTAP chloride (LIPOID), 0.2 mg Fe/ml oleic acid-coated iron oxide nanoparticles (ImagionBio) and 10 mM sodium citrate. 1 ml of 20 mg Fe/ml 15 nm diameter oleic acid-coated iron oxide nanoparticles in chloroform (ImagionBio, Lot #95-133) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams solanesol, 3.7 grams SPAN® 60, and 3 grams DOTAP were added to produce the oil phase. The oil phase was sonicated for 45 minutes in a 65 degree c. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber. The microfluidized formulation was terminally filtered with a 200 nm pore-size polyethersulfone (PES) filter and stored at 2 to 8 degrees C. Iron concentration was determined by ICP-OES. DOTAP and solanesol concentration were measured by RP-HPLC.

### Example 12: NP-7 Formulation (Prepared at 100 ml Scale)

NP-7 formulation comprises 37.5 mg/ml squalene (SEPPIC), 37 mg/ml SPAN® 60 (Millipore Sigma), 37 mg/ml TWEEN® 80 (Fisher Chemical), 30 mg/ml DOTAP chloride (LIPOID), 2.4 mg/ml Dynasan 114 (101 Oleo GmbH) and 10 mM sodium citrate. To a 200 ml beaker 3.75 grams squalene, 3.7 grams SPAN® 60, 0.24 grams Dynasan 114 and 3 grams DOTAP were added to produce the oil phase. The oil phase was sonicated for 45 minutes in a 65 degree c. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S) -reached 40-80 nm with a 0.1-0.3 polydispersity index (PDI). The microfluidized formulation was terminally filtered with a 200 nm pore-size polyethersulfone (PES) filter and stored at 2 to 8 degrees C. DOTAP and squalene concentration were measured by RP-HPLC.

### Example 13: NP-8 Formulation (Prepared at 100 ml Scale)

The NP-8 formulation comprises 43 mg/ml squalene (SEPPIC), 5 mg/ml SPAN® 85 (Millipore Sigma), 5 mg/ml TWEEN® 80 (Fisher Chemical), 4 mg/ml DOTAP chloride (LIPOID) and 10 mM sodium citrate. To a 200 ml beaker 4.3 grams squalene, 0.5 grams SPAN® 85, and 0.4 grams DOTAP were added to produce the oil phase. The oil phase was sonicated for 45 minutes in a 65 degree c. water bath. Separately, the aqueous phase was prepared by dissolving 2.6 grams TWEEN® 80 in 500 ml of 10 mM sodium citrate buffer prepared in nuclease free water. 95 ml of the aqueous phase was transferred to a separate glass bottle and heated to 65 degrees Celsius for 30 minutes. The oil phase was mixed with the 95 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H3OZ-200 μm ceramic interaction chamber until the z-average hydrodynamic diameter—measured by dynamic light scattering (Malvern Zetasizer Nano S) -reached 100±10 nm with a 0.05-0.1 polydispersity index (PDI). The microfluidized formulation was terminally filtered with a 200 nm pore-size polyethersulfone (PES) filter and stored at 2 to 8 degrees C. DOTAP and Squalene concentration were measured by RP-HPLC.

### Example 14: Lipid Nanoparticles and Cell-Based Assays for Evaluating Protein Production

The treatment groups were prepared. Eight of those groups were NanoLuc repRNA groups, with 600 ng dose per well was prepared using the Fe-lipid carrier, High Fe-lipid carrier, Fe-lipid carrier miglyol, High Fe-lipid carrier miglyol, Alum-lipid carrier, Fe-lipid carrier solanesol (SLN), NLC, and CNE formulations. The untreated group did not have NanoLuc. The various formulations were prepared by diluting NanoLuc repRNA to 8 ng/μL in 2.2 mL of RNAse-free water. The lipid carrier formulations and RNA master mix was complexed by adding 250 μL of each diluted formulation with 250 μL of diluted RNA, and mixed by pipetting up and down.

Cell transfections were carried out by seeding 7×10⁵ THP-1s per well in a 24-well plate. 80 micromolar (μM) PMA added to each well and incubated at 37° C.. The next day, the PMA-containing media was removed and replaced with complete RPMI (cRPMI) medium for one hour before transfection. The samples were then serially diluted in Opti-MEM™ (Thermo Fisher Scientific, Waltham, MA USA) to make a 10-point 1.5-fold dilution series starting at 0.45 ng/μL. The culture media was then removed from the plates by pipetting. 450 μL of Opti-MEM™ and 150 μL of the complexed formulation were added to the plate in duplicate. The empty wells were given 450 μL of Opti-MEM™ only. After four hours, the samples were removed from the plate by pipetting and replaced with 500 μL of growth media. The plate was then incubated overnight at 37° C.. The growth media was harvested the next day and stored at −80 degrees Celsius. Downstream assays were conducted and described below.

The luciferase assay was performed by first diluting the Nano-Glo® luciferase assay reagent 1:50 in buffer. 25 μL of supernatant was removed and mixed with 25 μL of Nano-Glo®reagent in a 96-well plate. This was incubated at room temperature for 3 minutes. The luminescence was read using a luminometer.

Next, an ELISA assay was performed to evaluate the TNF-alpha (a) protein levels in cell culture media using the human TNF-α DUOSET™ ELISA (R&D Systems) according to the manufacturer's protocol. The 96-well microplate was coated with anti-TNFα capture antibody. The plate was blocked and then media samples were added directly without dilution. After addition of the biotinylated detection antibody, SA-HRP, and substrate, the absorbance was read at 450 nm on a SPECTRAMAX® i3 (Molecular Devices) plate reader.

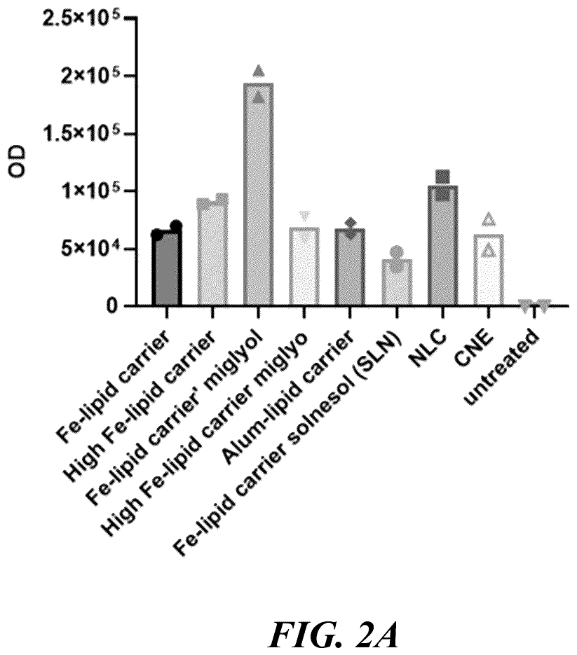

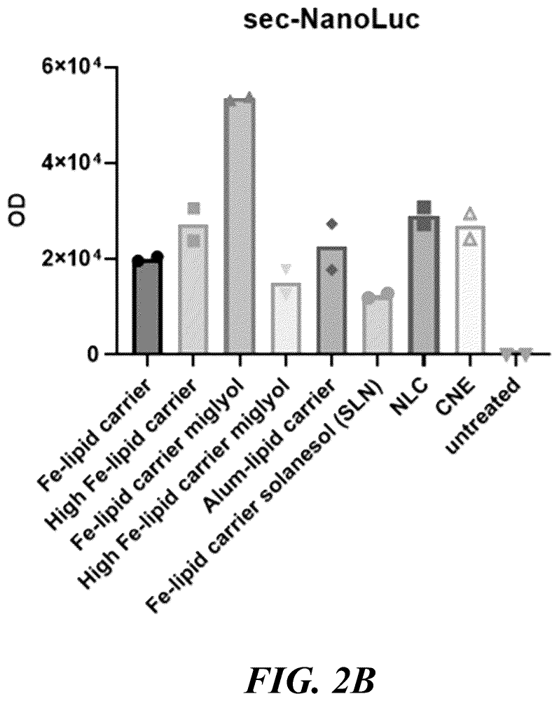

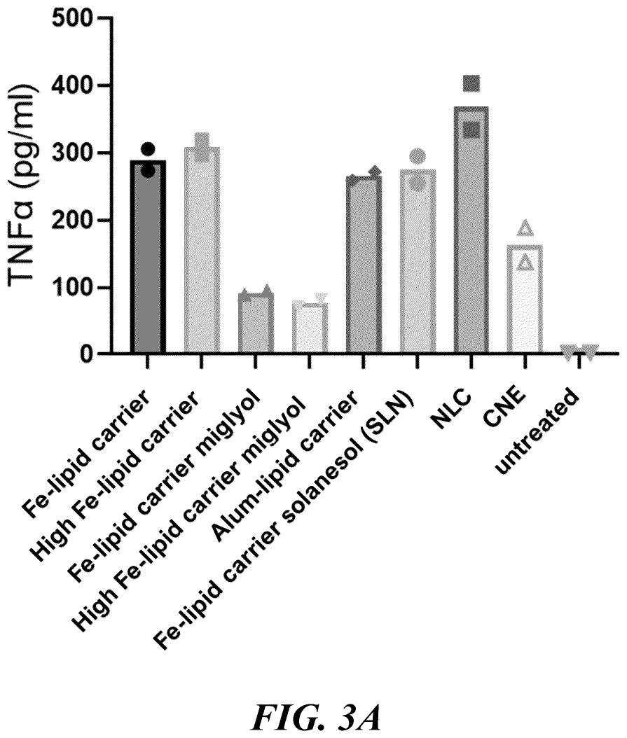

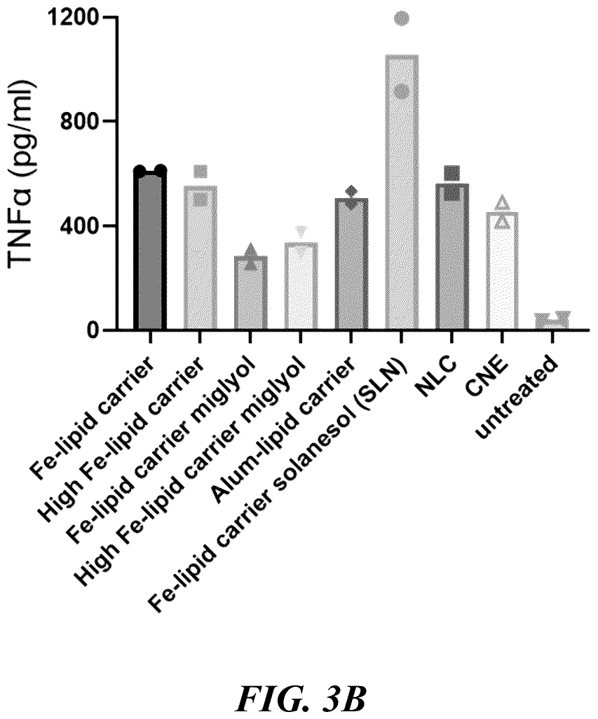

All studies in this example were done in duplicates. Results from the duplicates are presented as first experiment and second experiment respectively. The formulation comprising a lipid carrier and miglyol induced higher protein production off the replicon, as shown in the first experiment in FIG. 2A and in the second experiment in FIG. 2B. A reduced innate immune response was detected, as measured by TNF-α secretion (FIG. 3A-3B).

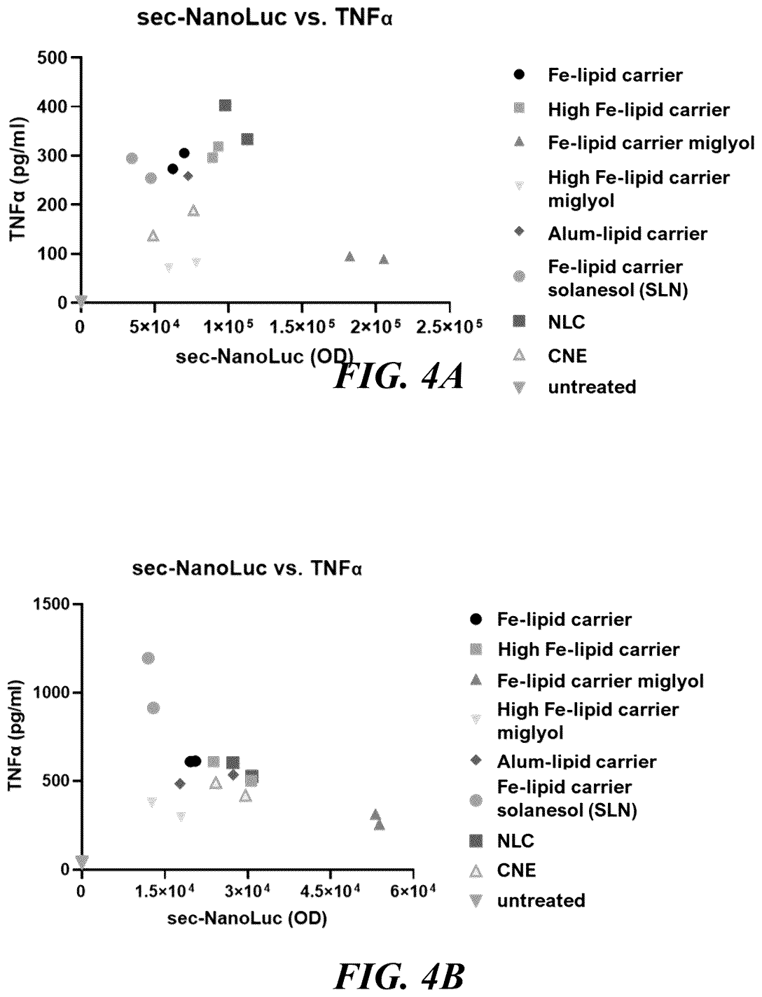

The correlation between enhanced protein production and low TNF-alpha stimulation was observed with the miglyol lipid carrier formulation, as shown in the first experiment in FIG. 4A and in the second experiment in FIG. 4B. The solanesol lipid carrier formulation induced slightly lower protein production, but a higher TNFα production (FIGS. 4A-4B). Correlation data from the first assay is shown in FIG. 4A are derived from the data represented in FIG. 2A and FIG. 3A. Correlation data from second assay is shown in FIG. 4B, which is derived from the data represented in FIG. 2B and FIG. 3B.

### Example 15: Exemplary Techniques and Materials for Producing Nanoparticles Described Herein

In a murine assay, C57BL/6 mice were inoculated as described in Table 6 below, after which secreted embryonic alkaline phosphatase (SEAP) levels were measured in serum. A summary of the materials used in the example is provided in Table 14.

TABLE 14
Materials.

| | | | Temperature/ |
|---|---|---|---|
| Description | Concentration | | Location |
| DNA encoding SEAP | 5 | mg/mL | −20° C. |
| (DNA sequence as set | | | |
| forth in SEQ ID | | | |
| NO: 79) | | | |
| repRNA encoding SEAP | 2217 | μg/mL | −80° C. |
| Lipid carrier | 30 | mg DOTAP/mL | 4° C. |
| formulation | | | |

TABLE 15
Treatment Groups.

| | | | | RNA | DNA | | Injection |
|---|---|---|---|---|---|---|---|
| | | | DNA/RNA- | dose | dose | | Volume |
| Group | n | Formulation | SEAP | [μg] | [μg] | N:P | [μL] |
| 1 | 5 | Naked | DNA-SEAP | | 20 | n/a | 50 |
| 2 | 5 | Lipid carrier | DNA-SEAP | | 10 | 15 | 50 |
| 3 | 5 | Lipid carrier | DNA-SEAP | | 10 | 7.5 | |
| 4 | 5 | Lipid carrier | DNA-SEAP | | 20 | 15 | |
| 5 | 5 | Lipid carrier | DNA-SEAP | | 20 | 7.5 | |
| 6 | 5 | Lipid carrier | RNA-SEAP | 1 | | 15 | 50 |
| 7 | 5 | Miglyol + lipid | RNA-SEAP | 1 | | 15 | 50 |
| | | carrier | | | | | |

Seven different formulations were prepared and administered intramuscularly across the seven treatment groups (Groups 1-7). DNA-SEAP or RNA-SEAP was diluted according to the volumes set forth in Table 16 to prepare the formulations for Groups 1-7. Table 16. Formulation Preparation; Dilution of RNA/DNA.

TABLE 16
Formulation Preparation; Dilution of RNA/DNA.

| | | DNA or | 40% | | |
|---|---|---|---|---|---|
| | DNA- or | RNA | sucrose | water | Total |
| Group | RNA-SEAP | [μL] | [μL] | [μL] | [μL] |
| 1 | DNA-SEAP | 40.0 | 125.0 | 85.0 | 250.0 |
| 2 | DNA-SEAP | 20.0 | 0.0 | 230.0 | 250.0 |
| 3 | DNA-SEAP | 20.0 | 0.0 | 230.0 | 250.0 |
| 4 | DNA-SEAP | 40.0 | 125.0 | 85.0 | 250.0 |
| 5 | DNA-SEAP | 40.0 | 0.0 | 210.0 | 250.0 |
| 6 | RNA-SEAP | 4.5 | 0.0 | 245.5 | 250.0 |
| 7 | RNA-SEAP | 4.5 | 0.0 | 245.5 | 250.0 |

The concentrations of diluted DNA or RNA prior to complexing With the lipid carrier was as follows (measured by NanoDrop spec): Groups 1, 4 and 5 contains about 820 μg/ml DNA; Groups 2 and 3 contained about 480 g/ml DNA; and Groups 6 and 7 contained about 43 g g/ml RNA.

Formulations for Groups 1-7 were diluted with 100 mM citrate as set forth in Table 17 below.

TABLE 17
Dilution of lipid carrier formulations.

| | | Lipid | 40% | 100 mM | | |
|---|---|---|---|---|---|---|
| | | Carrier | sucrose | citrate | Water | Total |
| Group | Formulation | [μl] | [μl] | [μl] | [μl] | [μl] |
| 1 | Naked | 0 | 0 | 30 | 270 | 300 |
| 2 | Lipid carrier | 120 | 150 | 30 | 0 | 300 |
| 3 | Lipid carrier | 60 | 150 | 30 | 60 | 300 |
| 4 | Lipid carrier | 240 | 0 | 30 | 30 | 300 |
| 5 | Lipid carrier | 120 | 150 | 30 | 0 | 300 |
| 6 | Lipid carrier | 12 | 150 | 30 | 108 | 300 |
| 7 | Miglyol + | 12 | 150 | 30 | 108 | 300 |
| | lipid carrier | | | | | |

The above formulations were complexed by adding 250.l diluted lipid carrier to 250.l diluted DNA or RNA. The resulting complexed formulations were incubated on ice for at least 30 minutes. Table 18 sets forth the experiment schedule for the assay.

TABLE 18
Experiment schedule.

| Day | Procedure | Notes on Mice |
|---|---|---|
| 0 | All inoculations | None |
| 4 | Bleed | Group 4 had ruffled fur, one mouse |
| | | emaciated (died during collection). |
| | | Hydropaque placed in cage. |
| 6 | Bleed | None |
| 8 | Bleed | None |
| 11 | Bleed | None |
| 14 | Bleed | None |

Mice were bled at regular intervals and serum was prepared immediately and stored at −80° C. until analyses for SEAP activity.

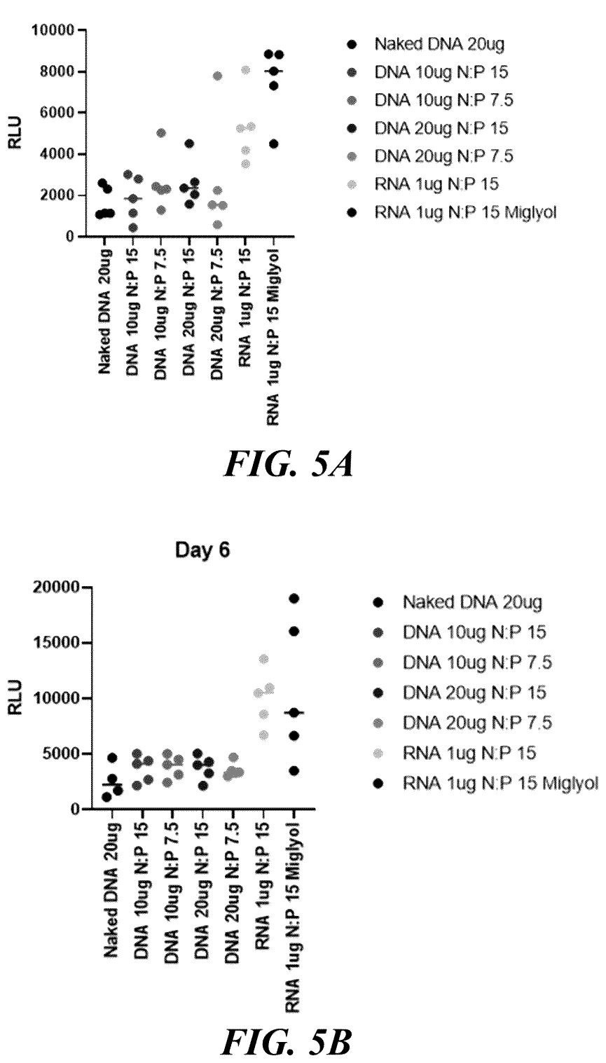

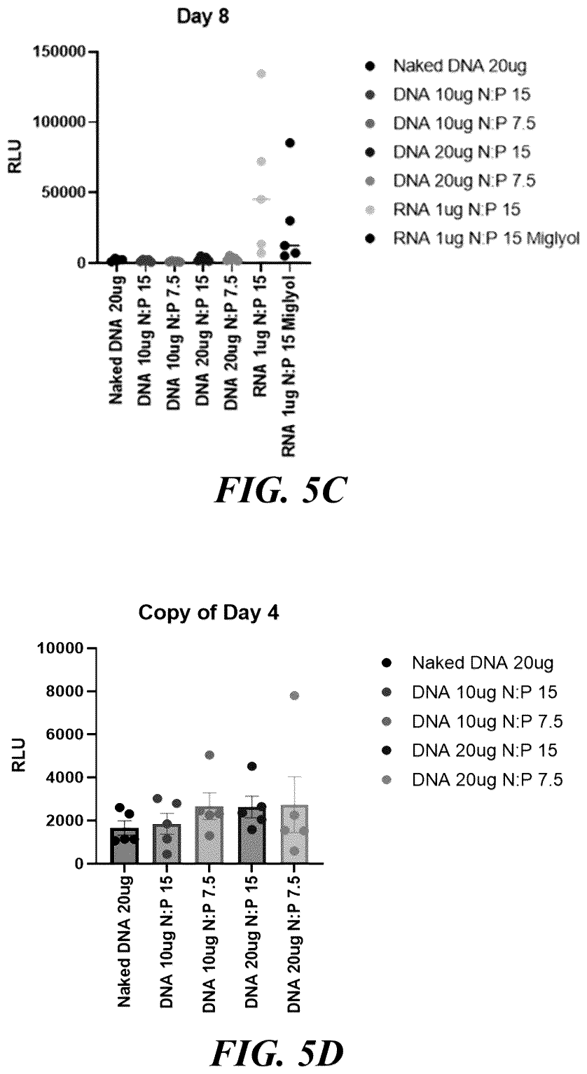

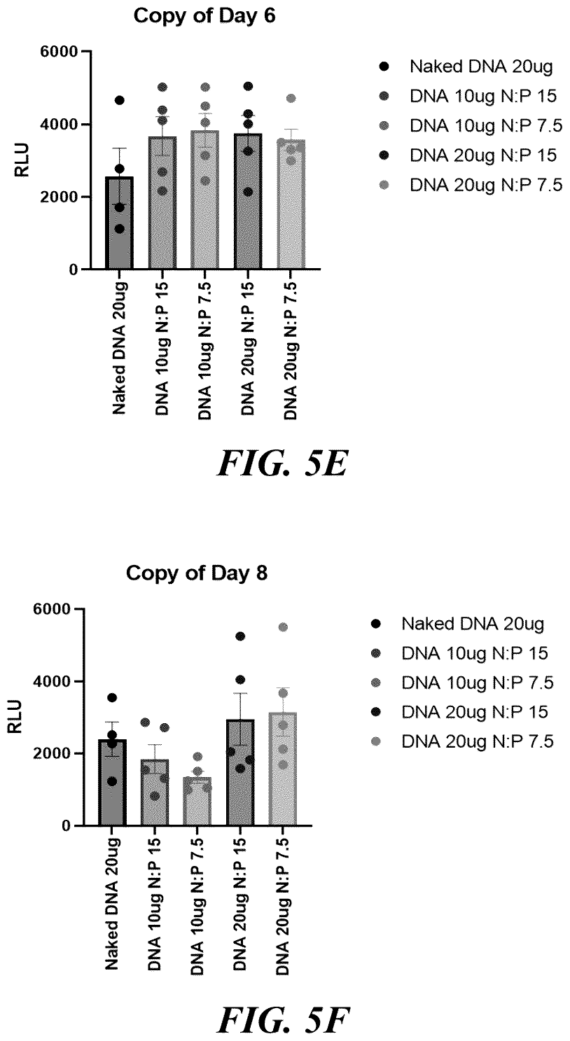

To evaluate SEAP levels in serum, all serum samples were thawed at the same time and SEAP detection was conducted. FIGS. 5A-5F illustrate the SEAP levels in BALB/c mice injected intramuscularly with varying iterations of lipid carrier-formulated DNA SEAP. Mice were bled at regular intervals, serum prepared and stored until analysis by SEAP assay. Data are displayed as mean and SE (n=5 per group).

As can be seen from FIGS. 5A-5F, lipid carrier formulations aid target protein production over delivery of DNA alone, particularly after day 6 following injection. Additionally, the data shows that inclusion of miglyol enhances protein production from an RNA replicon over lipid carrier formulations lacking miglyol.

### Example 16: Self-Replicating mRNA Construct

A plasmid encoding a T7 promoter followed by the 5′ and 3′ UTRs and nonstructural genes of Venezuelan equine encephalitis virus (VEEV) strain TC-83 was generated using standard DNA synthesis and cloning methods. The VEEV replicon mRNA backbone is set forth in SEQ ID NO: 71.

### Example 17: Additional Nanoparticle Formulations

Additional nanoparticle formulations are produced according to the following tables (Table 19 and Table 10). The mRNA comprises a sequence encoding the TRP-1 tumor associated antigen with a VEEV replicon mRNA backbone (SEQ ID NO: 71).

TABLE 19
mRNA Vaccine Formulation.
Dosage form:
Solution for Injection
Composition:

| Each 0.5 ml Vial | | | Concentration |
|---|---|---|---|
| Contains: | Quantity | | (mg/ml) |
| mRNA | 25 | mcg | 0.05 |
| DOTAP | 0.75 | mg | 1.5 |
| Iron Oxide Nanoparticles | 0.005 | mg | 0.01 |
| Squalene | 0.94 | mg | 1.88 |
| Sorbitan Monostearate | 0.93 | mg | 1.86 |
| Polysorbate 80 | 0.93 | mg | 1.86 |
| Sucrose IP | 50 | mg | 100 |
| Citric Acid Monohydrate | 1.05 | mg | 2.1 |

| | |
|---|---|
| Water for Injection | q.s. to 0.5 ml |

TABLE 10
Lyophilized mRNA Vaccine Formulation.
Dosage form:
Lyophilized powder
Composition:

| | | | Concen- | Approximate |
|---|---|---|---|---|
| Each 5 dose | | | tration | dry |
| vial contains: | Quantity | | (mg/ml) | weight % |
| mRNA | 50 | mcg | 0.02 | 0.02 |
| DOTAP | 1.5 | mg | 0.6 | 0.57 |
| Squalene | 1.88 | mg | 0.752 | 0.72 |
| Sorbitan Monostearate | 1.86 | mg | 0.744 | 0.71 |
| Polysorbate 80 | 1.86 | mg | 0.744 | 0.71 |
| Sucrose IP | 250 | mg | 100 | 95.3 |
| Citric Acid | 5.25 | mg | 2.1 | 2 |
| Monohydrate | | | | |

| Water for Injection (for | 2.5 ml |
|---|---|
| reconstitution) | |

### Example 18: TRP-1 Replicon Prevents B16FO Tumor Growth

B16 subcutaneous melanoma mouse models were used in the assays provided herein. Upon subcutaneous injection, B16 animals will form a palpable tumor in approximately 5 to 10 days and grow to approximately a 1×1×1-cm tumor in approximately 14 to 21 days.

A lipid carrier and a TRP-1 RNA replicon were generated (SEQ ID NO: 76). The amino acid sequence of the TRP-1 is SEQ ID NO: 78.

Female C57BL/6 mice were immunized by intramuscular injection of repRNA-TRP1 at either a 0.2 mcg or 1 mcg dose, on either one or two occasions. Table 11 provides the assaycondiue

TABLE 11
Immunization groups and conditions.

| | | | | | Volume | |
|---|---|---|---|---|---|---|
| Group | n | Immunization | Day 0 | Day 14 | [μl] | Route |
| 1 | 5 | Lipid carrier + 0.2 μg TRP-1 repRNA | yes | yes | 50 | i.m. |
| 2 | 5 | Lipid carrier + 0.2 μg TRP-1 repRNA | — | yes | 50 | i.m. |
| 3 | 5 | Lipid carrier + 1 μg TRP-1 repRNA | yes | yes | 50 | i.m. |
| 4 | 5 | Lipid carrier + 1 μg TRP-1 repRNA | — | yes | 50 | i.m. |
| 5 | 5 | none | — | — | 50 | — |

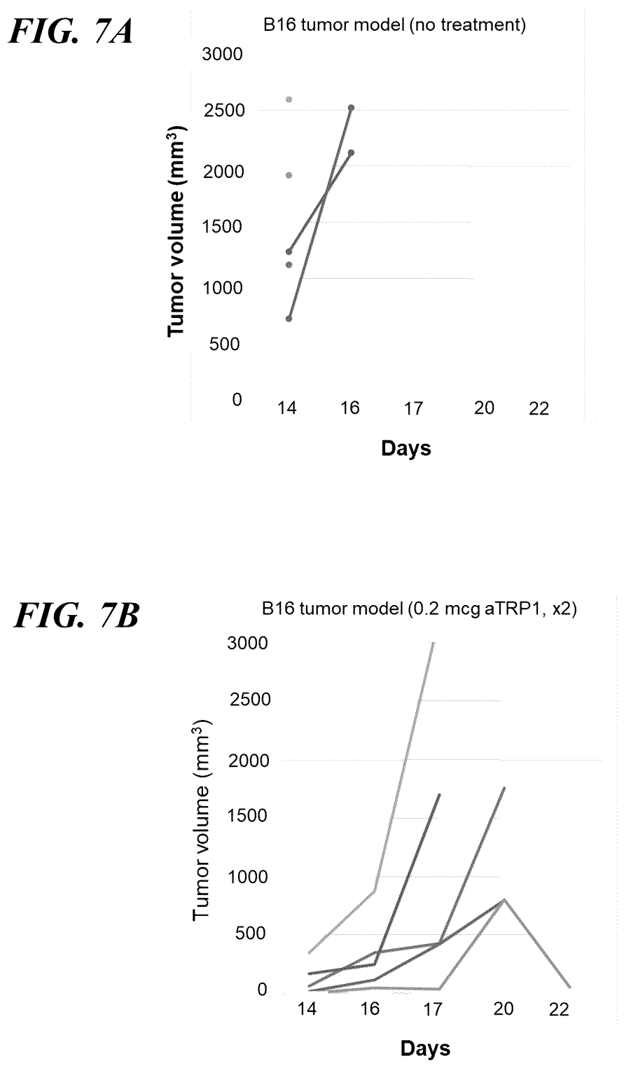

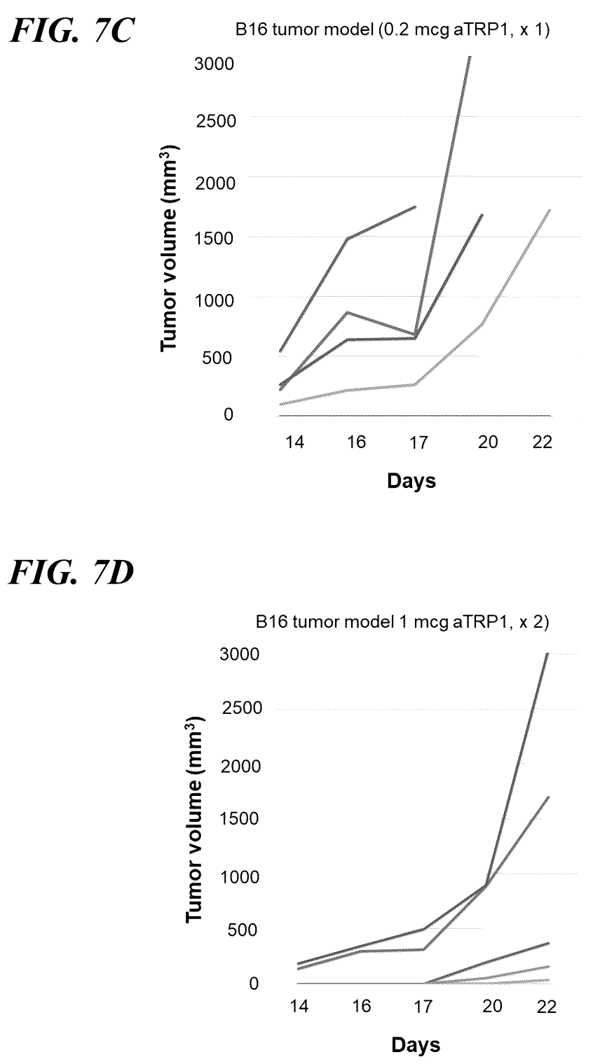

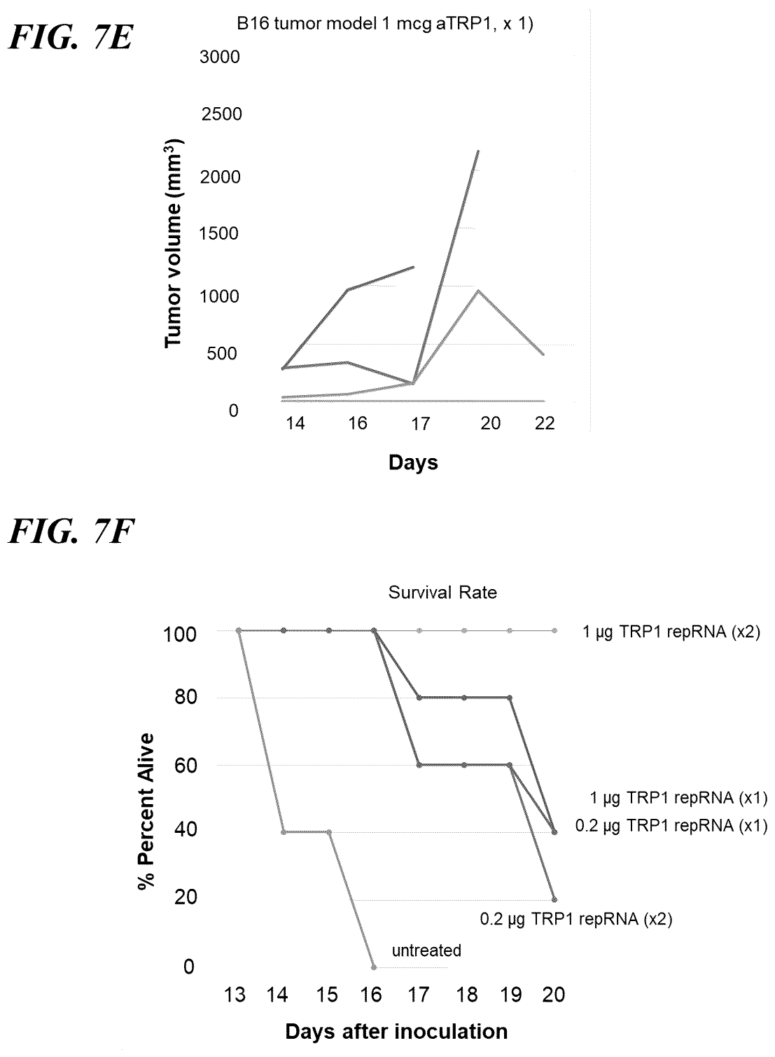

Blood was collected 2 weeks after completion of immunization regimen, serum prepared for each animal and antibodies binding TRP1 assessed by ELISA. n=5 per group. Results are presented as tumor growth for each individual animal (FIGS. 7A-7E) and as survival (FIG. 7F). Immunization with TRP1-expressing replicon limits B16 outgrowth in a dose dependent manner.

Next, the therapeutic efficacy of TRP-1 repRNA vaccinations were investigated at various doses and treatment regimens as shown in Table 12.

TABLE 12
Immunization groups and conditions.

| | Group | | Lipid | | | |
|---|---|---|---|---|---|---|
| | # | N | Carrier | Dose (RNA) | Regimen | Time/Tumor size |
| Arm 1 | 1 | 5 | NP-1 | 0 μg (control) | — | — |
| | 2 | 5 | NP-1 | 1 μg | Weekly | Pre-palpable (day 3) |
| | 3 | 5 | NP-1 | 5 μg | Weekly | Pre-palpable (day 3) |
| | 4 | 5 | NP-1 | 1 μg | Weekly | Palpable 3-4 mm (day 7-9) |
| | 5 | 5 | NP-1 | 5 μg | Weekly | Palpable 3-4 mm (day 7-9) |
| Arm 2 | 6 | 5 | NP-1 | 5 μg scrambled | Weekly | — |
| | | | | RNA (control) | | |
| | 7 | 5 | NP-1 | 1 μg | Single | Pre-palpable (day 3) |
| | 8 | 5 | NP-1 | 5 μg | Single | Pre-palpable (day 3) |
| | 9 | 5 | NP-1 | 1 μg | Single | Palpable 3-4 mm (day 7-9) |
| | 10 | 5 | NP-1 | 5 μg | Single | Palpable 3-4 mm (day 7-9) |

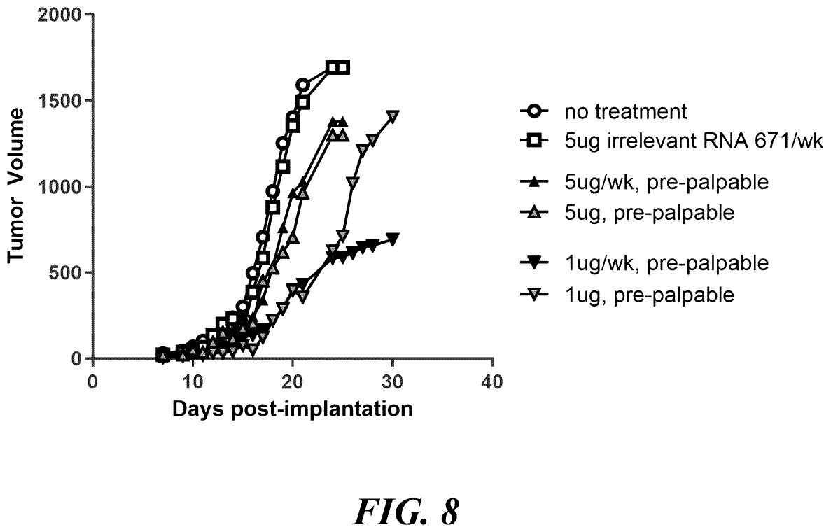

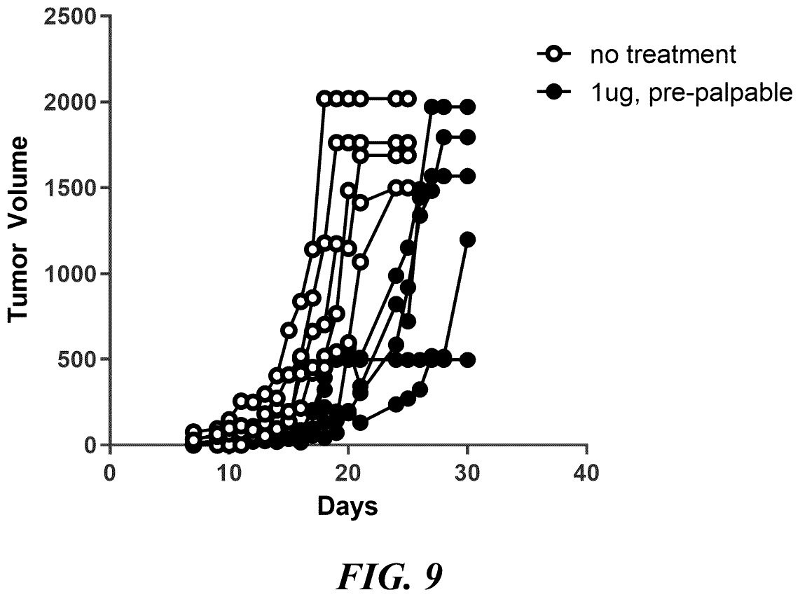

Female C57BL/6 mice were immunized by intramuscular injection of repRNA-TRP1 at either a 1 mcg or 5 mcg dose, beginning on day 3 (pre-palpable) or day 7 (post-palpable) after tumor cell implantation. Immunizations were repeated weekly thereafter. n=5 per group. Results are presented as mean tumor growth for each group (FIG. 8) and for each individual animal (FIG. 9). The results further confirm that immunization with TRP 1-expressing replicon limits B 16 outgrowth.

### Example 19: MAGE-A1 Replicon Induces Antigen-Specific T Cells

A lipid nanoparticle carrier and a MAGE-A1 RNA replicon were generated (SEQ ID NO: 75). The amino acid sequence of the MAGE-A1 is SEQ ID NO: 77.

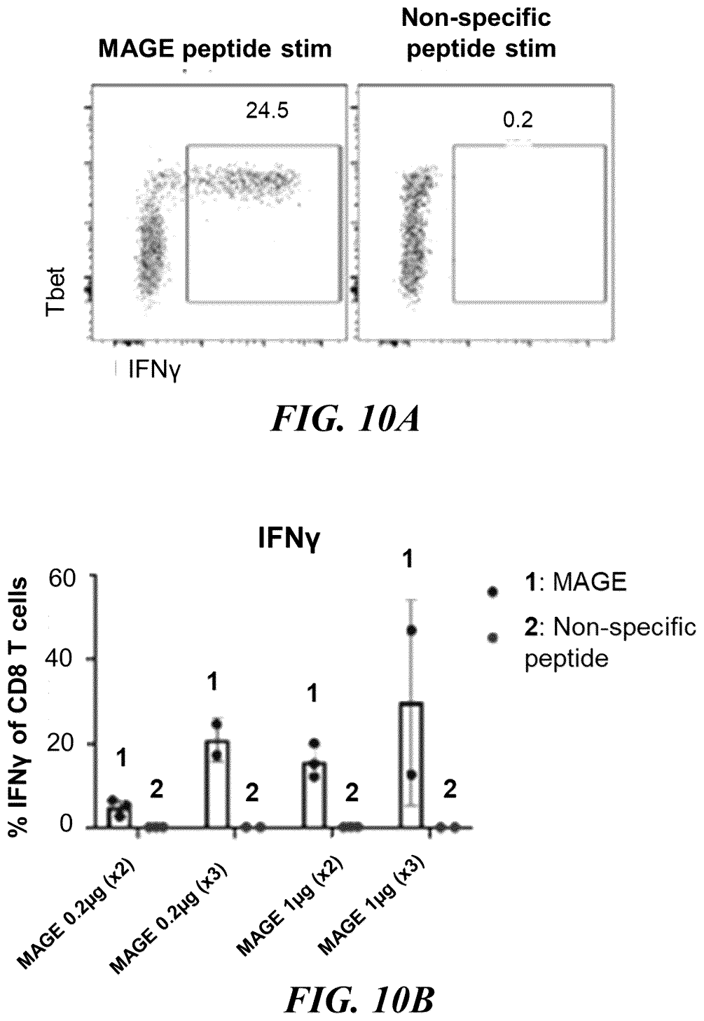

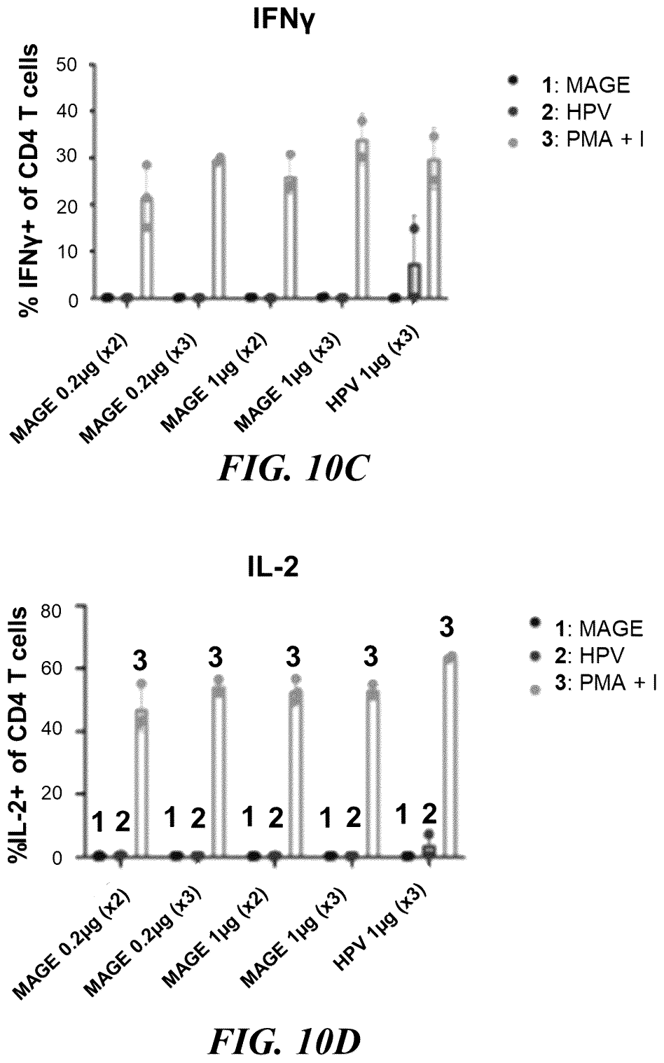

Female C57BL/6 mice were immunized by intramuscular injection of a repRNA-MAGE-A1 at either 0.2 or 1 mcg dose. Immunizations were performed on day 0, 14 and 80 (3x) or day 14 and 80 (2x), then spleens collected on day 91 and single cell suspensions prepared. Cells were then incubated with MAGE-A1 or a HPV E6 (non-specific) peptide pool, and subjected to flow cytometry. CD8 T cells expressing Tbet and producing IFN-gamma (γ) (FIGS. 10A-10B) and CD4 T cells producing IFNγ or IL-2 (FIGS. 10C-10D) are shown. Immunization with MAGE-expressing replicon induces antigen-specific T cells.

### Example 20: TRP-1 and MAGE-A3 Combination Therapy Reduces Tumor Volume

A lipid nanoparticle carrier was combined with a MAGE-A3 RNA replicon and a TRP-1 RNA replicon. The amino acid sequence of the TRP-1 is SEQ ID NO: 78 The amino acid sequence of the MAGE-A3 is SEQ ID NO: 87.

B16 subcutaneous melanoma mouse models were used in the assays provided herein according to Example 17. The therapeutic efficacy of TRP-1 and MAGE-A3 repRNA vaccinations were investigated at various doses and treatment regimens as shown in Table 12.

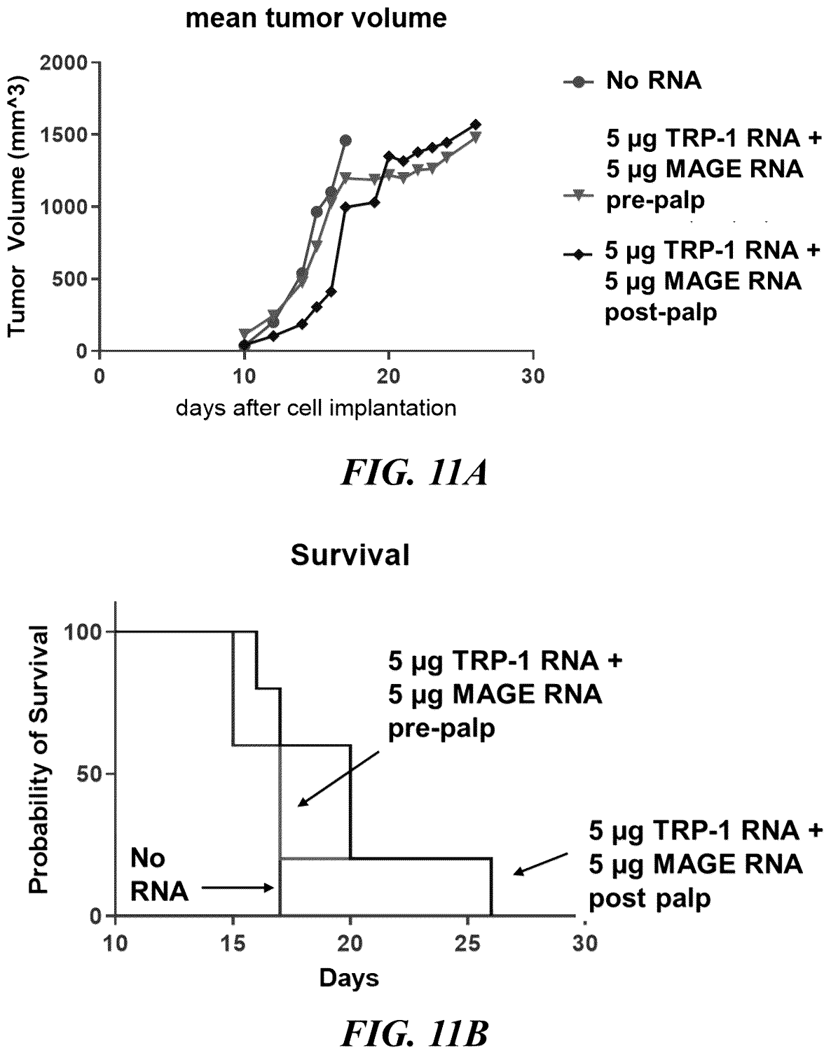

Tumor volume was quantified (FIG. 11A) for control, NP-1+TRP-1+MAGE-A3 pre-palpable animals, and NP-1+TRP-1+MAGE-A3. The probability of survival increased for animals treated with the lipid carrier and TRP-1 and MAGE-A3 encoding repRNAs relative to untreated animals (FIG. 11B).

### Example 21: Self-Replicating RNA Prostein Construct

A lipid nanoparticle carrier was combined with a prostein-encoding RNA replicon. The RNA encoding for the prostein protein encodes for an amino acid sequence of SEQ ID NO: 90. The VEEV replicon mRNA backbone sequence is set forth in SEQ ID NO: 71.

What is claimed is:

1. A composition comprising:
lipid nanoparticles, wherein the lipid nanoparticles comprise:
surfactants, wherein the surfactants comprise:
a cationic lipid;
a hydrophilic surfactant; and
a hydrophobic surfactant; and
at least one nucleic acid, wherein the at least one nucleic acid comprises a sequence encoding for a cancer-associated protein region comprising a cell membrane-contacting domain or a functional fragment thereof.
